(19) United States
(12) Patent Application Publication
(10) Pub. No.: US 2024/0285676 A1
(43) Pub. Date: Aug. 29, 2024

(54) BOTTLEBRUSH POLYMERS AND METHODS THEREOF

(71) Applicant: Regents of the University of Minnesota, Minneapolis, MN (US)

(72) Inventors: Joseph Franklin Hassler, Minneapolis, MN (US); Adelyn Crabtree, Minneapolis, MN (US); Benjamin Hackel, Minneapolis, MN (US); Frank S. Bates, Minneapolis, MN (US); Timothy P. Lodge, Minneapolis, MN (US); Joseph M. Metzger, Minneapolis, MN (US)

(21) Appl. No.: 18/438,184

(22) Filed: Feb. 9, 2024

Related U.S. Application Data
(60) Provisional application No. 63/444,855, filed on Feb. 10, 2023.

(51) Int. Cl. A61K 31/77; A61P 9/00; A61P 21/00; C08G 61/08; C08G 65/08; C08G 83/00

(52) U.S. Cl. CPC A61K 31/77; A61P 9/00; A61P 21/00; C08G 61/08; C08G 65/08; C08G 83/005; C08G 2261/126; C08G 2261/132; C08G 2261/3324; C08G 2261/354; C08G 2261/418; C08G 2261/75

(57) ABSTRACT

The present disclosure relates to bottlebrush polymers and methods thereof. Methods can include methods of stabilizing an interface, stabilizing a cell, or treating a disease, disorder, or condition using an effective amount of the bottlebrush polymer.

### CROSS-REFERENCE TO RELATED APPLICATION

This application claims the benefit of U.S. Provisional Patent Application No. 63/444,855, filed on Feb. 10, 2023, which is incorporated by reference herein in its entirety.

### STATEMENT OF GOVERNMENT SUPPORT

This invention was made with government support under HL122323, and AR071349 awarded by the National Institutes of Health. The government has certain rights in the invention.

### TECHNICAL FIELD

This disclosure relates to bottlebrush polymers and methods thereof.

### BACKGROUND

Block polymer amphiphiles in aqueous environments can assemble into diverse micelle and vesicle morphologies, with applications in numerous fields including pharmaceutical delivery and as nanoreactors. Additionally, single chains or unimers can interact with phospholipid bilayers in therapeutically relevant ways, ranging from membrane solubilization to stabilization. In particular, poloxamers are a specific class of linear block polymer amphiphiles of poly(ethylene oxide) (PEO) and poly(propylene oxide) (PPO) that are commercially available (e.g., Pluronics®) and are biocompatible. Yet, the stabilization mechanism of poloxamer-lipid interactions is still not fully understood.

### SUMMARY

This disclosure generally relates to bottlebrush polymers and uses thereof. In some cases, bottlebrush polymers (BBPs) refer to a class of macromolecules having polymeric side chains attached to a linear polymeric backbone (e.g., a rigid backbone). Various properties of the BBP can be controlled, such as molecular weight, side chain type (e.g., poloxamer-derived side chains, such as ethylene oxide, propylene oxide, or polymeric forms thereof), side chain hydrophobicity versus hydrophilicity, side chain length, side chain grafting density, backbone type, backbone length, block or segment length, copolymer type (e.g., block versus statistical copolymer), and the like. In turn, such properties can be tuned to provide desired self-assembly or solubilization characteristics. Accordingly, described herein are methods for tuning such properties to provide BBPs for stabilizing interfaces.

In some cases, high molecular weight, high grafting density, and/or low dispersity polymers (e.g., BBPs) are desired. For instance and without limitation, hydrogenation reactions can be conducted to minimize alkene α-chain end impurities within the side chain, and polymerization reactions can be conducted to avoid chain transfer/coupling side reactions. Taken together, methods including such reactions can provide BBPs having high molecular weight (e.g., a number average molecular weight (Mₙ) of 10-300 kDa), high grafting density (e.g., a grafting density of 0-100% of repeat units bearing a polymeric side chain), and/or low dispersity (e.g., a molecular weight distribution (Ð) <1.20).

In one aspect, the present disclosure provides a polymer comprising a structure of Formula I:

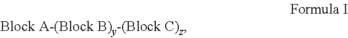

wherein:
each of Block A, Block B, and Block C of the polymer, independently, comprises a backbone polymer comprising one or more repeating units, wherein at least one of the one or more repeating units is covalently linked to a side chain; and
each of y and z is, independently, 0 or 1;
wherein:
   a first side chain of Block A is a hydrophilic side chain;
   a second side chain of Block B is a hydrophobic side chain; and
   a third side chain of Block C is a hydrophilic side chain that is different from the first side chain; and
wherein the polymer is characterized by a molecular weight distribution (Ð) of less than about 1.5, 1.2, 1.15, or 1.1.

In some embodiments, Block A comprises poly(ethylene oxide). In some embodiments, Block B comprises poly(propylene oxide).

In some embodiments, the polymer is characterized by one or more of the following:
a number average molecular weight (Mₙ) from about 20 to 400 kDa;
the backbone polymer in the polymer is present in an amount from about 5 to 25 wt. %;
a number of repeating units in the Block A is from about 5 to 200;
a number of repeating units in the Block B is from about 5 to 50;
a number of repeating units in the Block C is from about 5 to 200;
the hydrophilic side chain in the polymer is present in an amount from about 50 to 95 wt. %; and/or
the hydrophobic side chain in the polymer is present in an amount from about 0 to 50 wt. %.

In some embodiments, at least one of the one or more repeating units comprises:

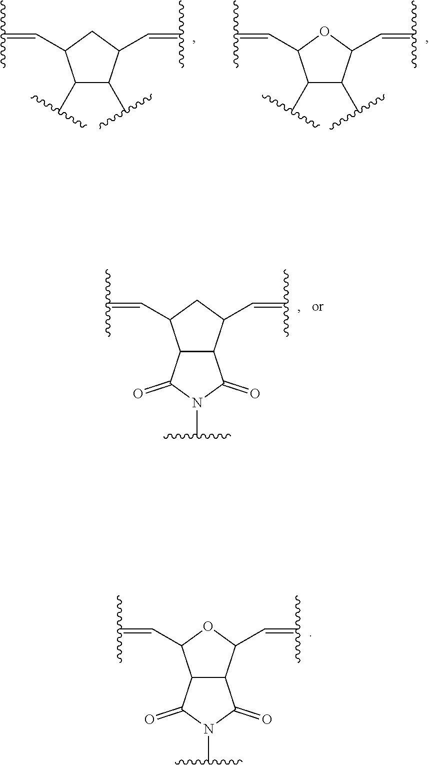

In some embodiments, Block A and/or Block B further comprises repeating units that are covalently linked to a polymeric side chain. In some embodiments, the polymeric side chain of Block A comprises a hydrophilic polymeric side chain. In some embodiments, the polymeric side chain of Block B comprises a hydrophobic polymeric side chain. In some embodiments, Block A and/or Block C further comprises repeating units that are not covalently linked to a polymeric side chain.

In some embodiments, at least one of the repeating units that are not covalently linked to the polymeric side chain comprises:

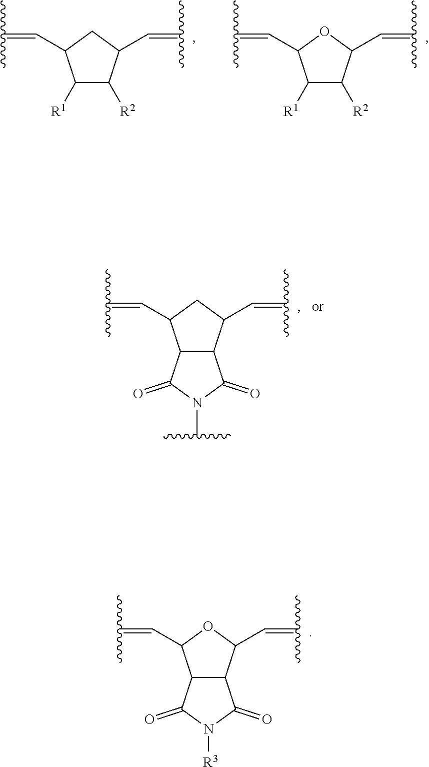

wherein:
each of R¹ and R² is, independently, hydrogen, C₁₋₆ alkyl, or —C(═O)—O—R^{A};
each R^{A} of R¹ and R² is, independently, hydrogen, aryl (e.g., phenyl), heteroaryl, C₁₋₆ alkyl, C₁₋₆ alkyl-aryl (e.g., alkyl-phenyl), aryl-C₁₋₆ alkyl, C₁₋₆ alkyl-heteroaryl, or heteroaryl-C₁₋₆ alkyl; and
R³ is hydrogen, aryl (e.g., phenyl), heteroaryl, C₁₋₆ alkyl, C₁₋₆ alkyl-aryl (e.g., C₁₋₆ alkyl-phenyl), aryl-C₁₋₆ alkyl, C₁₋₆ alkyl-heteroaryl, or heteroaryl-C₁₋₆ alkyl.

In some embodiments, at least one of the repeating units that are covalently linked to the side chain comprises:

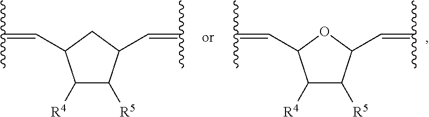

wherein:
each R⁴ is, independently, hydrogen, C₁₋₆ alkyl, the hydrophilic side chain, or the hydrophobic side chain;

each R⁵ is, independently, the hydrophilic side chain or the hydrophobic side chain.

In some embodiments, the hydrophilic side chain comprises

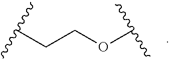 [SMILES: *CCO*]

In some embodiments, the hydrophilic side chain comprises poly(ethylene oxide).

In some embodiments, the hydrophobic side chain comprises

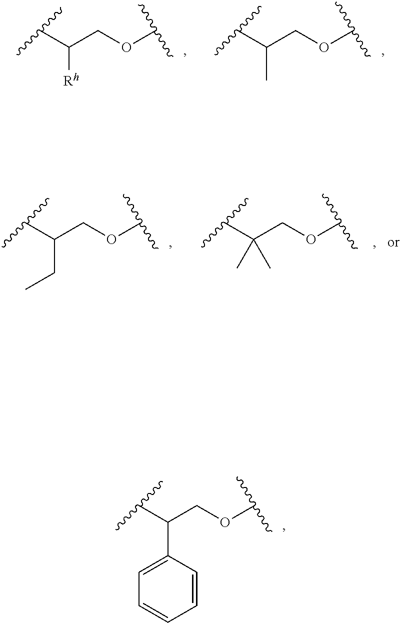

wherein R^{h} is aryl (e.g., phenyl), heteroaryl, C₁₋₆ alkyl, C₁₋₆ alkyl-aryl (e.g., alkyl-phenyl), aryl-C₁₋₆ alkyl, C₁₋₆ alkyl-heteroaryl, or heteroaryl-C₁₋₆ alkyl.

In some embodiments, the hydrophobic side chain comprises poly(propylene oxide). In some embodiments, the hydrophilic side chain and/or the hydrophobic side chain does not include C₂₋₈ alkenyl. In some embodiments, at least one of the repeating units are covalently linked to the side chain through a linker. In some embodiments, the linker comprises —C(═O)—O—, —O—C(═O)—, —C(═O)—, or a covalent bond. In some embodiments, Block A comprises more repeating units than a number of repeating units present in Block B.

In some embodiments, the polymer comprises a structure of Formula II:

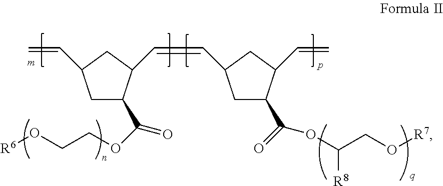

wherein:
each R⁶ is, independently, hydrogen or C₁₋₆ alkyl;
each R⁷ is, independently, hydrogen, C₁₋₁₂ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
each R⁸ is, independently, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
m is 10-200; n is 5-200; p is 5-50; and q is 5-200.

In some embodiments, R⁷ is or comprises

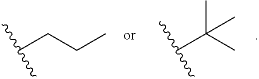

In some embodiments, the polymer comprises a structure of Formula IA:

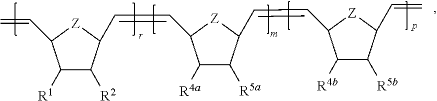

wherein:
Z is oxy (—O—) or C₁₋₆ alkylene (e.g., methylene);
each of R¹ and R² is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R¹ and R², when taken together, form an anhydride moiety or a dicarboximide moiety;
each of R⁴a and R⁴b is, independently, hydrogen, C₁₋₆ alkyl, the hydrophilic side chain, or the hydrophobic side chain;
each of R⁵a and R⁵b is, independently, the hydrophilic side chain or the hydrophobic side chain;
m is 10-200; p is 0-50; and r is 0-180.

In some embodiments, Z is methylene, R⁵a is the hydrophilic side chain, and R⁵b is the hydrophobic side chain.

The present disclosure also provides a composition comprising a polymer (e.g., any described herein) and a pharmaceutically acceptable excipient. In some embodiments, the composition further comprises a therapeutic agent (e.g., any described herein).

The present disclosure also provides a method of stabilizing an interface. In one aspect, the method comprises delivering a polymer (e.g., any described herein) to a solution comprising the interface. In some embodiments, the solution further comprises an aqueous solvent, a therapeutic agent, a lipid, or a combination thereof.

The present disclosure also provides a method of stabilizing a cell. In one aspect, the method comprises contacting a polymer (e.g., any described herein) with a cell or a portion thereof. In some embodiments, the cell or the portion thereof comprises a cell membrane or a lipid layer. In some embodiments, the polymer is provided at a concentration from about 0.001 mg/mL to 30 mg/mL.

The present disclosure also provides a method of treating a disease, a disorder, or a condition. In one aspect, the method comprises administering a therapeutically effective amount of a polymer (e.g., any described herein) to a subject in need thereof. In some embodiments, the disease, the disorder, or the condition is selected from the group consisting of skeletal muscle disorder, sickle cell disease, reperfusion disease or reperfusion injury, ischemia, and cardiovascular disease. Other diseases, disorders, and conditions are described herein. In some embodiments, the therapeutically effective amount of the polymer solution is provided as a pharmaceutical composition. Optionally, the pharmaceutical composition can further include comprises a pharmaceutically acceptable excipient, a pharmaceutically acceptable carrier, an aqueous solvent, a therapeutic agent, a lipid, or a combination thereof.

The present disclosure also provides a method of preparing a monomer (e.g., a macromonomer) of Formula III:

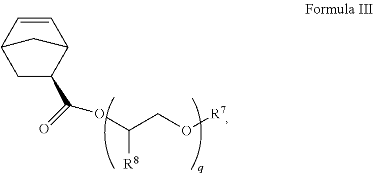

the method comprising:
(i) forming a reaction mixture comprising an alkoxide of R^{7a}OH (e.g., potassium tert-butoxide) and an epoxide of

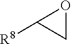

under conditions suitable to prepare a compound of Formula IIIA and a compound of Formula IIIB:

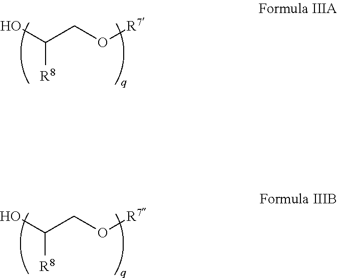

wherein:
each R⁷a is, independently, C₃₋₁₂ alkyl (e.g., an unbranched or branched alkyl);
each R⁸ is, independently, hydrogen, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
R⁷′ does not include alkenyl;
R⁷″ comprises alkenyl or alkynyl;
each of R⁷′ and R⁷″ is a reacted form of R^{7a}; and
q is 5-200;

(ii) forming a reaction mixture comprising a compound of Formula IIIA, a compound of Formula IIIB, and a hydrogenation catalyst (e.g., palladium on carbon) under conditions suitable to prepare a compound of Formula IIIA and a compound of Formula IIIC:

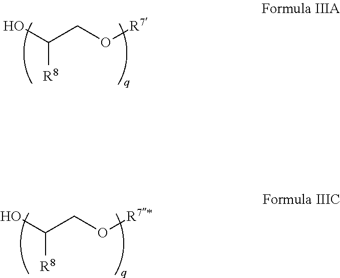

wherein R⁷″* comprises an unsaturated version of R⁷″;

(iii) forming a reaction mixture comprising a compound of Formula IIIA, a compound of Formula IIIC, and a compound of

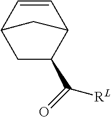

under conditions suitable to prepare a compound of Formula III;
wherein:
   each R⁷ is, independently, independently, C₃₋₁₂ alkyl (e.g., an unbranched or branched alkyl, such as

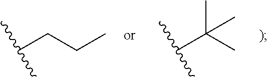

and
R^{L} is a leaving group (e.g., halo, hydroxyl, alkoxy, and the like).

In some embodiments, Formula IIIA and Formula IIIB are as follows:

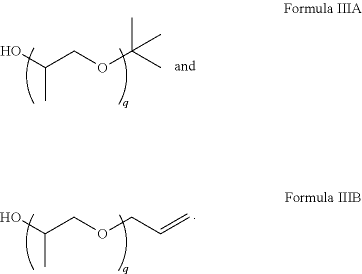

In some embodiments, Formula IIIC is as follows:

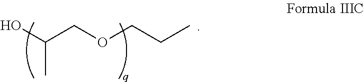

In some embodiments, the method further comprises preparing a polymer of Formula II:

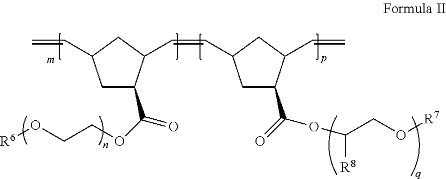

the method comprising:
(i-a) forming a reaction mixture comprising the monomer (e.g., a macromonomer) of Formula III and a catalyst under suitable conditions to prepare a compound of Formula IIA:

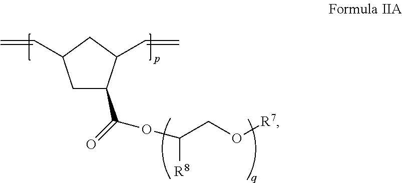

and
(ii-a) forming a reaction mixture comprising a compound of Formula IIA, a macromonomer of

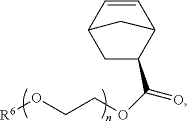

and a catalyst under suitable conditions to prepare a compound of Formula II; wherein each R⁶ is, independently, hydrogen or C₁₋₆ alkyl; m is 10-200; n is 5-200; and p is 5-50.

In some embodiments, (i-a) and/or (ii-a) is performed to provide more than about 80% conversion within a period of about less than about ten minutes.

In some embodiments, (i-a) and/or (ii-a) further comprises providing a monomer of Formula IVA or IVB, or an isomers thereof, thereby forming a modified form of a compound of Formula IIA or a compound of Formula II having a diluent:

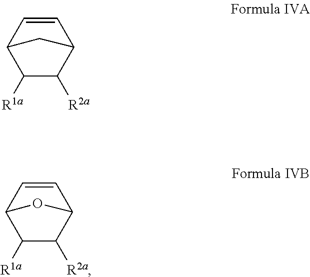

wherein:
each of R¹a and R²a is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R^{1a} and R^{2a}, when taken together, form an anhydride moiety or a dicarboximide moiety.

### Definitions

By “attach,” “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, x bond interactions, hydrophobic interactions, inclusion complexes, clathration, van der Waals interactions, and combinations thereof.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof (e.g., cycloaliphatic), and further including straight- and branched-chain arrangements, and all stereo and position isomers as well.

By “alkenyl” or “alkene” is meant an optionally substituted C₂₋₂₄ alkyl group having one or more double bonds, as well as a compound having such a group. In some cases, the unsaturated monovalent hydrocarbon can be derived from removing one hydrogen atom from one carbon atom of a parent alkene. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl.

By “alkoxy” is meant —OR, where R is an optionally substituted aliphatic group or an optionally substituted alkyl group, as described herein. Exemplary alkoxy groups include, but are not limited to, methoxy, ethoxy, n-propoxy, isopropoxy, n-butoxy, t-butoxy, sec-butoxy, n-pentoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₂₋₈ alkenyl; (2) C₂₋₈ alkynyl; (3) C₁₋₆ alkoxy (e.g., -OR^{Y}); (4) C₁₋₆ alkylsulfinyl (e.g., —S(O)R^{Y}); (5) C₁₋₆ alkylsulfonyl (e.g., —SO₂R^{Y}); (6) amino; (7) aryl (e.g., C₄₋₁₈ aryl); (8) arylalkoxy (e.g., —OR^{Z}Ar^{Z}); (9) aryloyl (e.g., —C(O)Ar^{Z}); (10) azido (—N₃); (11) carboxyaldehyde (—C(O)H); (12) carboxyl (—C(O)OH); (13) C₃₋₈ cycloalkyl; (14) halo; (15) heterocyclyl (e.g., C₁₋₁₈ heterocyclyl including one or more heteroatoms, such as N, O, S, and P); (16) heterocyclyloxy (e.g., —OHet^{Z}); (17) heterocyclyloyl (e.g., —C(O)Het^{Z}); (18) hydroxyl; (19) N-protected amino; (20) nitro (—NO₂); (21) oxo (═O); (22) C₃₋₈ spirocyclyl; (23) C₁₋₆ thioalkoxy (e.g., —SR); (24) thiol (—SH); (25) —CO₂R^{A}, where R^{A} is selected from the group consisting of (a) hydrogen, (b) C₁₋₆ alkyl, (c) C₄₋₁₈ aryl, and (d) C₁₋₆ alk-C₄₋₁₈ aryl; (26) —C(O)NR^{B}R^{C}, where each of R^{B} and R^{C} is, independently, selected from the group consisting of (a) hydrogen, (b) C₁₋₆ alkyl, (c) C₄₋₁₈ aryl, and (d) C₁₋₆ alk-C₄₋₁₈ aryl; (27) —SO₂R^{D}, where R^{D} is selected from the group consisting of (a) C₁₋₆ alkyl, (b) C₄₋₁₈ aryl, and (c) C₁₋₆ alk-C₄₋₁₈ aryl; (28) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; and (29) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃₋₈ cycloalkyl, and (i) C₁₋₆ alk-C₃₋₈ cycloalkyl, where in one case, no two groups are bound to the nitrogen atom through a carbonyl group or a sulfonyl group (e.g., where R^{Y} is alkyl (e.g., C₁₋₆ alkyl), R^{Z} is alkylene (e.g., C₁₋₆ alkylene), Ar^{Z} is aryl (e.g., C₄₋₁₈ aryl), and Het^{Z} is heterocyclyl (e.g., C₁₋₁₈ heterocyclyl including one or more heteroatoms, such as N, O, S, and P), as defined herein). The alkyl group can be a primary, secondary, or tertiary alkyl group substituted with one or more substituents (e.g., one or more halo or alkoxy). In some cases, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkyl-aryl” is meant -Ar-Ak, where Ar is an optionally substituted arylene group and Ak is an optionally substituted alkyl group, as described herein. The arylene and alkyl groups can be substituted or unsubstituted. For example, the arylene and alkyl groups can be substituted with one or more substitution groups, as described herein for aryl or alkyl. Exemplary unsubstituted alkyl-aryl groups include C₁₋₁₂ alkyl-C₄₋₁₈ aryl, C₁₋₆ alkyl-C₄₋₁₈ aryl, and C₁₋₃ alkyl-C₄₋₁₈ aryl groups.

By “alkyl-heteroaryl” is meant -Het-Ak, where Het is an optionally substituted heteroarylene group and Ak is an optionally substituted alkyl group, as described herein. The heteroarylene and alkyl groups can be substituted or unsubstituted. For example, the heteroarylene and alkyl groups can be substituted with one or more substitution groups, as described herein for aryl or alkyl. Exemplary unsubstituted alkyl-heteroaryl groups include C₁₋₁₂ alkyl-C₁₋₁₈ heteroaryl, C₁₋₆ alkyl-C₁₋₁₈ heteroaryl, and C₁₋₃ alkyl-C₁₋₁₈ heteroaryl groups.

By “alkylene” is meant a multivalent (e.g., bivalent, trivalent, tetravalent, etc.) form of an aliphatic group or an alkyl group, as described herein. Exemplary alkylene groups include methylene, ethylene, propylene, butylene, etc. In some cases, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkoxycarbonyl” is meant —C(O)—O-Ak, where Ak is an optionally substituted aliphatic group or optionally substituted alkyl group, as described herein. The aliphatic or alkyl group can be substituted or unsubstituted. For example, the aliphatic or alkyl group can be substituted with one or more substitution groups, as described herein for alkyl. Exemplary unsubstituted alkoxycarbonyl group include C₂₋₁₈, C₂₋₁₄, C₂₋₁₂, C₂₋₁₀, C₂₋₈, C₂₋₆, C₃₋₁₈, C₃₋₁₄, C₃₋₁₂, or C₃₋₁₀ alkoxycarbonyl groups.

By “alkynyl” or “alkyne” is meant an unsaturated monovalent hydrocarbon having at least two carbon atom to 50 carbon atoms (C₂₋₅₀) and at least one carbon-carbon triple bond, as well as a compound having such a group. In some cases, the unsaturated monovalent hydrocarbon can be derived from removing one hydrogen atom from one carbon atom of a parent alkyne. An alkynyl group can be branched, straight-chain, or cyclic (e.g., cycloalkynyl); and can include two to 25 carbon atoms (C₂₋₂₅) or two to ten carbon atoms (C₂₋₁₀). An exemplary alkynyl includes an optionally substituted C₂₋₂₄ alkyl group having one or more triple bonds. The alkynyl group can be cyclic or acyclic and is exemplified by ethynyl, 1-propynyl, and the like. The alkynyl group can be monovalent or multivalent (e.g., bivalent) by removing one or more hydrogens to form appropriate attachment to the parent molecular group or appropriate attachment between the parent molecular group and another substitution. The alkynyl group can also be substituted or unsubstituted. For example, the alkynyl group can be substituted with one or more substitution groups, as described herein for alkyl.

By “anhydride” is meant a compound or a moiety having a —C(O)—O—C(O)— group. This group can be attached to different atoms on the same functional group (e.g., the same cyclic, aromatic, aryl, or heteroaryl group) or to different functional groups.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized re-electron system. Typically, the number of out of plane re-electrons corresponds to the Huckel rule (4n+2). The point of attachment to the parent structure typically is through an aromatic portion of the condensed ring system. The term “aromatic” also includes “heteroaromatic,” as described herein.

By “aryl” is meant a group that contains any carbon-based aromatic group including, but not limited to, benzyl, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)Ak, in which Ak is an alkyl group, as defined herein); (2) C₁₋₆ alkyl; (3) C₁₋₆ alkoxy (e.g., —OAk, in which Ak is an alkyl group, as defined herein); (4) C₁₋₆ alkoxy-C₁₋₆ alkyl (e.g., an alkyl group, which is substituted with an alkoxy group —OAk, in which Ak is an alkyl group, as defined herein); (5) C₁₋₆ alkylsulfinyl (e.g., —S(O)Ak, in which Ak is an alkyl group, as defined herein); (6) C₁₋₆ alkylsulfinyl-C₁₋₆ alkyl (e.g., an alkyl group, which is substituted by an alkylsulfinyl group —S(O)Ak, in which Ak is an alkyl group, as defined herein); (7) C₁₋₆ alkylsulfonyl (e.g., —SO₂Ak, in which Ak is an alkyl group, as defined herein); (8) C₁₋₆ alkylsulfonyl-C₁₋₆ alkyl (e.g., an alkyl group, which is substituted by an alkylsulfonyl group —SO₂Ak, in which Ak is an alkyl group, as defined herein); (9) aryl; (10) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., meant an alkyl group, as defined herein, substituted by an amino group); (12) heteroaryl; (13) C₁₋₆ alk-C₄₋₁₈ aryl (e.g., —A^{L}Ar, in which A^{L} is an alkylene group and Ar is an aryl group, as defined herein); (14) aryloyl (e.g., —C(O)Ar, in which Ar is an aryl group, as defined herein); (15) azido (e.g., an —N₃ group); (16) cyano (e.g., a —CN group); (17) C₁₋₆ azidoalkyl (e.g., a —N₃ azido group attached to the parent molecular group through an alkyl group, as defined herein); (18) carboxyaldehyde (e.g., a —C(O)H group); (19) carboxyaldehyde-C₁₋₆ alkyl (e.g., —A^{L} C(O)H, in which A^{L} is an alkylene group, as defined herein); (20) C₃₋₈ cycloalkyl; (21) C₁₋₆ alk-C₃₋₈ cycloalkyl (e.g., —A^{L}Cy, in which A^{L} is an alkylene group and Cy is a cycloalkyl group, as defined herein); (22) halo (e.g., F, Cl, Br, or I); (23) C₁₋₆ haloalkyl (e.g., an alkyl group, as defined herein, substituted with one or more halo); (24) heterocyclyl; (25) heterocyclyloxy (e.g., —OHet, in which Het is a heterocyclyl group); (26) heterocyclyloyl (e.g., —C(O)Het, in which Het is a heterocyclyl group); (27) hydroxyl (e.g., a —OH group); (28) C₁₋₆ hydroxyalkyl (e.g., an alkyl group, as defined herein, substituted by one to three hydroxyl groups, with the proviso that no more than one hydroxyl group may be attached to a single carbon atom of the alkyl group); (29) nitro (e.g., an —NO₂ group); (30) C₁₋₆ nitroalkyl (e.g., an alkyl group, as defined herein, substituted by one to three nitro groups); (31) N-protected amino; (32) N-protected amino-C₁₋₆ alkyl; (33) oxo (e.g., an ═O group); (34) C₁₋₆ thioalkoxy (e.g., -SAK, in which Ak is an alkyl group, as defined herein); (35) thio-C₁₋₆ alkoxy-C₁₋₆ alkyl (e.g., an alkyl group, which is substituted by an thioalkoxy group -SAK, in which Ak is an alkyl group, as defined herein); (36) —(CH₂)ᵣCO₂R^{A}, where r is an integer of from zero to four, and R^{A} is selected from the group consisting of (a) hydrogen, (b) C₁₋₆ alkyl, (c) C₄₋₁₈ aryl, and (d) C₁₋₆ alk-C₄₋₁₈ aryl; (37) —(CH₂),CONR^{B}R^{C}, where r is an integer of from zero to four and where each R^{B} and R^{C} is independently selected from the group consisting of (a) hydrogen, (b) C₁₋₆ alkyl, (c) C₄₋₁₈ aryl, and (d) C₁₋₆ alk-C₄₋₁₈ aryl; (38) (CH₂)ᵣSO₂R^{D}, where r is an integer of from zero to four and where R^{D} is selected from the group consisting of (a) C₁₋₆ alkyl, (b) C₄₋₁₈ aryl, and (c) C₁₋₆ alk-C₄₋₁₈ aryl; (39) (CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; (40) —(CH₂)ᵣNR^{G}R^{H}, where r is an integer of from zero to four and where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃₋₈ cycloalkyl, and (i) C₁₋₆ alk-C₃₋₈ cycloalkyl, wherein in one case, no two groups are bound to the nitrogen atom through a carbonyl group or a sulfonyl group; (41) thiol; (42) perfluoroalkyl (e.g., an alkyl group, as defined herein, having each hydrogen atom substituted with a fluorine atom); (43) perfluoroalkoxy (e.g., —OR_{f}, in which R_{f} is an alkyl group, as defined herein, having each hydrogen atom substituted with a fluorine atom); (44) aryloxy (e.g., —OAr, where Ar is an optionally substituted aryl group, as described herein); (45) cycloalkoxy (e.g., —OCy, in which Cy is a cycloalkyl group, as defined herein); (46) cycloalkylalkoxy (e.g., —OA^{L}Cy, in which A^{L} is an alkylene group and Cy is a cycloalkyl group, as defined herein); and (47) arylalkoxy (e.g., —OA^{L}Ar, in which A^{L} is an alkylene group and Ar is an aryl group, as defined herein). In particular cases, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “aryl-alkyl” is meant -Ak-Ar, where Ak is an optionally substituted alkylene group and Ar is an optionally substituted aryl group, as described herein. The alkylene and aryl groups can be substituted or unsubstituted. For example, the alkylene and aryl groups can be substituted with one or more substitution groups, as described herein for aryl or alkyl. Exemplary unsubstituted aryl-alkyl groups include C₄₋₁₈ aryl-C₁₋₁₂ alkyl, C₄₋₁₈ aryl-C₁₋₆ alkyl, and C₄₋₁₈ aryl-C₁₋₃ alkyl groups.

By “arylene” is meant a multivalent (e.g., bivalent, trivalent, tetravalent, etc.) form of an aromatic group or aryl group, as described herein. Exemplary arylene groups include phenylene, naphthylene, biphenylene, triphenylene, diphenyl ether, acenaphthenylene, anthrylene, or phenanthrylene. In some cases, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for alkyl or aryl.

By “aryloxycarbonyl” is meant —C(O)—O—Ar, where Ar is an optionally substituted aromatic group or optionally substituted aryl group, as described herein. The aromatic or aryl group can be substituted or unsubstituted. For example, the aromatic or aryl group can be substituted with one or more substitution groups, as described herein for alkyl or aryl. Exemplary unsubstituted aryloxycarbonyl group include C₃₋₁₈, C₃₋₁₄, C₃₋₁₂, C₃₋₁₀, C₅₋₁₈, C₅₋₁₄, C₅₋₁₂, C₅₋₁₀, C₇₋₁₈, C₇₋₁₄, C₇₋₁₂, or C₇₋₁₀ aryloxycarbonyl groups.

By “carbonyl” and the suffix “oyl” is meant —C(O)—.

By “dicarboximide” is meant a compound or a moiety having a —C(O)—NR^{N1}—C(O)— group, in which R^{N1} is H, optionally substituted aliphatic, or optionally substituted aromatic. This group can be attached to different atoms on the same functional group (e.g., the same cyclic, aromatic, aryl, or heteroaryl group) or to different functional groups.

By “epoxy,” “epoxide,” or “oxiranyl,” is meant

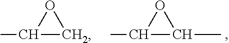

or a compound including such a group, in which one or more hydrogen atoms can be optionally substituted with another functional group. An example of an epoxy can include RCHOCH₂, in which R is selected from aliphatic, heteroaliphatic, aromatic, as defined herein, or any combination thereof. Another example of an epoxy can include RCHOCHR′, in which each of R and R′ is, independently, selected from aliphatic, heteroaliphatic, aromatic, as defined herein, or any combination thereof; or R and R′, taken together with the carbon atom to which each are attached, form a cycloalkyl group, as defined herein. Optional substitutions for hydrogen atoms can include an alkyl group and the like.

By “halo” is meant F, Cl, Br, or I.

By “haloalkyl” is meant is meant an optionally substituted alkyl, as defined herein, having one or more halo substituents.

By “heteroaliphatic” is meant an aliphatic group, as defined herein, including at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

By “heteroalkyl,” “heteroalkenyl,” and “heteroalkynyl” is meant an alkyl, alkenyl, or alkynyl group (which can be branched, straight-chain, or cyclic), respectively, as defined herein, including at least one heteroatom to 20 heteroatoms, such as one to 15 heteroatoms, or one to 5 heteroatoms, which can be selected from, but not limited to, oxygen, nitrogen, sulfur, silicon, boron, selenium, phosphorous, and oxidized forms thereof within the group.

By “heteroalkylene” is meant an alkylene group, as defined herein, containing one, two, three, or four non-carbon heteroatoms (e.g., independently selected from the group of nitrogen, oxygen, phosphorous, sulfur, silicon, halo, or a combination of any of these).

By “heteroaromatic” is meant a subset of heterocyclyl groups, which are aromatic, i.e., they contain 4n+2 pi electrons within the mono- or multicyclic ring system; or a subset of aromatic groups, as defined herein, having one or more heteroatoms, i.e., they contain one or more heteroatoms within the mono- or multicyclic ring system. Heterocyclyl groups include cyclic groups having one or more heteroatoms.

By “heteroaryl” is meant an aryl group including at least one heteroatom to six heteroatoms, such as one to four heteroatoms, which can be selected from, but not limited to, oxygen, nitrogen, sulfur, silicon, boron, selenium, phosphorous, and oxidized forms thereof within the ring. Such heteroaryl groups can have a single ring or multiple condensed rings, where the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, aromatic, other functional groups, or any combination thereof. For example, the heteroaryl group can be substituted with one or more substitution groups, as described herein for alkyl or aryl. An exemplary heteroaryl includes a subset of heterocyclyl groups, as defined herein, which are aromatic, i.e., they contain 4n+2 pi electrons within the mono- or multicyclic ring system.

By “heteroarylene” is meant a multivalent (e.g., bivalent, trivalent, tetravalent, etc.) form of a heteroaryl group, as described herein. The heteroarylene group can be branched or unbranched. The heteroarylene group can also be substituted or unsubstituted. For example, the heteroarylene group can be substituted with one or more substitution groups, as described herein for alkyl or aryl.

By “heteroaryl-alkyl” is meant -Ak-Het, where Ak is an optionally substituted alkylene group and Het is an optionally substituted heteroaryl group, as described herein. The alkylene and heteroaryl groups can be substituted or unsubstituted. For example, the alkylene and heteroaryl groups can be substituted with one or more substitution groups, as described herein for aryl or alkyl. Exemplary unsubstituted heteroaryl-alkyl groups include C₁₋₁₈ heteroaryl-C₁₋₁₂ alkyl, C₁₋₁₈ heteroaryl-C₁₋₆ alkyl, and C₁₋₁₈ heteroaryl-C₁₋₃ alkyl groups.

By “heteroatom” is meant an atom other than carbon, such as oxygen, nitrogen, sulfur, silicon, boron, selenium, or phosphorous. In particular disclosed cases, such as when valency constraints do not permit, a heteroatom does not include a halogen atom.

By “hydroxyl” is meant —OH.

By “salt” is meant an ionic form of a compound or structure (e.g., any formulas, compounds, or compositions described herein), which includes a cation or anion compound to form an electrically neutral compound or structure. Salts (e.g., simple salts having binary compounds, double salts, triple salts, etc.) are well known in the art. For example, salts are described in Berge S M et al., “Pharmaceutical salts,” J. Pharm. Sci. 1977 January;66(1): 1-19; International Union of Pure and Applied Chemistry, “Nomenclature of Inorganic Chemistry,” Butterworth & Co. (Publishers) Ltd., London, England, 1971 (2nd ed.); and in “Handbook of Pharmaceutical Salts: Properties, Selection, and Use,” Wiley-VCH, April 2011 (2nd rev. ed., eds. P. H. Stahl and C. G. Wermuth). The salts can be prepared in situ during the final isolation and purification of the compounds of the disclosure or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecyl sulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

By “pharmaceutically acceptable salt” is meant a salt that is, within the scope of sound medical judgment, suitable for use in contact with the tissues or cells of humans and animals without undue toxicity, irritation, allergic response and the like and are commensurate with a reasonable benefit/risk ratio.

By “pharmaceutically acceptable excipient” is meant any ingredient other than a compound or structure (e.g., any formulas, compounds, or compositions described herein) and having the properties of being non-toxic and non-inflammatory in a subject. Exemplary, non-limiting excipients include adjuvants, antiadherents, antioxidants, binders, carriers, coatings, compression aids, diluents, disintegrants, dispersing agents, dyes (colors), emollients, emulsifiers, fillers (diluents), film formers or coatings, flavors, fragrances, glidants (flow enhancers), isotonic carriers, lubricants, preservatives, printing inks, solvents, sorbents, stabilizers, suspensing or dispersing agents, surfactants, sweeteners, waters of hydration, or wetting agents. Any of the excipients can be selected from those approved, for example, by the United States Food and Drug Administration or other governmental agency as being acceptable for use in humans or domestic animals. Exemplary excipients include, but are not limited to alcohol, butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, glycerol, hydroxypropyl cellulose, hydroxypropyl methylcellulose, lactated Ringer's solution, lactose, magnesium stearate, maltitol, maltose, mannitol, methionine, methylcellulose, methyl paraben, microcrystalline cellulose, polyethylene glycol, polyol, polyvinyl pyrrolidone, povidone, pregelatinized starch, propyl paraben, retinyl palmitate, Ringer's solution, shellac, silicon dioxide, sodium carboxymethyl cellulose, sodium chloride injection, sodium citrate, sodium starch glycolate, sorbitol, starch (corn), stearic acid, stearic acid, sucrose, talc, titanium dioxide, vegetable oil, vitamin A, vitamin E, vitamin C, water, and xylitol.

By “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, any formulas, compounds, or compositions described herein for modulating any target described herein or treating any disease described herein. Each carrier must is “acceptable” in the sense of being compatible with the other ingredients of the formulation and not injurious to the patient. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Non-limiting examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain additives such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid, and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters), and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, “administration,” “administering,” and variants thereof refers to introducing a compound or a composition into a subject and includes concurrent and sequential introduction of a compound or a composition. “Administration” can refer, e.g., to therapeutic, pharmacokinetic, diagnostic, research, placebo, and experimental methods. “Administration” also encompasses in vitro and ex vivo treatments. The introduction of a compound or a composition into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the compound or the composition to perform its intended function.

As used herein, the terms “subject,” “individual,” and “patient” are used interchangeably herein and refer to any animal subject for whom diagnosis, treatment, or therapy is desired, particularly humans. In some cases, the subject is a mammal (e.g., a human subject). In some cases, the subject is a non-human mammal (e.g., mouse, rat, guinea pig, dog, cat, horse, cow, pig, rabbit, sheep, or non-human primate, such as a monkey, chimpanzee, or baboon).

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the terms “therapeutically effective amount” and “effective amount” are used interchangeably to refer to an amount of a compound or a composition that is sufficient to provide the intended benefit (e.g., prevention, prophylaxis, delay of onset of symptoms, or amelioration of symptoms of a disease). In prophylactic or preventative applications, an effective amount can be administered to a subject susceptible to, or otherwise at risk of developing a disease, disorder, or condition to eliminate or reduce the risk, lessen the severity, or delay the onset of the disease, disorder, or condition, including a biochemical, histologic and/or behavioral symptoms of the disease, disorder, or condition, its complications, and intermediate pathological phenotypes. An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Disclosed compositions and methods include those that exhibit large therapeutic indices. A therapeutically effective dose can be estimated initially from cell culture assays.

Also, a dose can be formulated in animal models to achieve a circulating plasma concentration range that includes the IC50 (i.e., the concentration of the agent for modulating a target, such as any described herein), which achieves a half-maximal inhibition of symptoms) as determined in cell culture, or in an appropriate animal model. Levels in plasma can be measured, for example, by high performance liquid chromatography or other assay. The effects of any particular dosage can be monitored by a suitable bioassay. The dosage can be determined by a physician and adjusted, as necessary, to suit observed effects of the treatment.

As used herein, the terms “treat,” “treating,” and/or “treatment” include abrogating, substantially inhibiting, slowing, or reversing the progression of a disorder, disease, or condition, substantially ameliorating clinical symptoms of a disorder, disease, or condition, or substantially preventing the appearance of clinical symptoms of a disorder, disease, or condition, obtaining beneficial or desired clinical results. Treating further refers to accomplishing one or more of the following: (a) reducing the severity of the disorder, disease or condition); (b) limiting development of symptoms characteristic of the disorder, disease, or condition(s) being treated; (c) limiting worsening of symptoms characteristic of the disorder, disease, or condition(s) being treated; (d) limiting recurrence of the disorder, disease, or condition(s) in subjects that have previously had the disorder, disease, or condition(s); and (e) limiting recurrence of symptoms in subjects that were previously asymptomatic for the disorder, disease, or condition(s). Beneficial or desired clinical results, such as pharmacologic and/or physiologic effects include, but are not limited to, preventing the disease, disorder, or condition from occurring in a subject predisposed to the disease, disorder, or condition but does not yet experience or exhibit symptoms of the disease (prophylactic treatment); alleviation of symptoms of the disease, disorder, or condition, diminishment of extent of the disease, disorder, or condition; stabilization (e.g., not worsening) of the disease, disorder, or condition; preventing spread of the disease, disorder, or condition, delaying or slowing of the disease, disorder, or condition progression; amelioration or palliation of the disease, disorder, or condition; and combinations thereof, as well as prolonging survival as compared to expected survival if not receiving treatment.

The details of one or more cases of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1A shows decay curves for L-E₉₃P₅₄E₉₃ polymer-only (14 μM) and polymer+POPC (14 μM polymer+875 μM POPC). FIG. 1B shows decay curves for B-E₁₀⁴³P₅¹⁵ polymer-only (14 μM) and polymer+POPC (14 μM polymer+875 μM POPC) at the same conditions as in FIG. 1A. FIG. 1C shows a summary of the number fraction of chains in each possible state, determined by the sum of exponentials model. Error bars are the standard deviation of three independent measurements.

FIG. 2A shows a comparison of PFG-NMR decay curves for L-E₉₃P₅₄E₉₃ and a PEO homopolymer of similar size. No binding was observed for the PEO homopolymer. FIG. 2B shows a comparison of PFG-NMR decay curves for B-E₁₀⁴³P₆¹⁵ and B-E₁₅⁴³, a bottlebrush PEO homopolymer with an identical backbone degree of polymerization. No binding was observed for the bottlebrush PEO homopolymer. Error bars are the standard deviation of three independent measurements.

FIG. 3 shows PFG-NMR decay curves for B-E₁₀⁴³P₆¹⁵ (Mₙ=26 kDa) and B-E₂₁⁴³P₁₁¹⁵ (Mₙ=55 kDa). Error bars are the standard deviation of at least two replicates.

FIG. 4A shows PFG-NMR decay curves of two BBPs with similar molecular weights and compositions but different PEO blocks. B-E₁₁⁴³P₆¹⁵ is star-like with a short backbone and long side chains (lower schematic pointing to circle symbols in graph), while B-E₄₀¹⁰P₇¹⁵ is brush-like with a long backbone and short side chains (upper schematic pointing to diamond symbols in graph). FIG. 4B shows schematics depicting a possible effect of side chain length (flexibility) on binding to lipid bilayers that is consistent with the data shown in FIG. 4A.

FIG. 5A shows PFG-NMR decay curves for block and statistical copolymers with longer PEO side chains (N_{sc, PEO}=43) than PPO side chains (N_{sc, PPO}=15). FIG. 5B shows PFG-NMR decay curves for block and statistical copolymers with shorter PEO side chains (N_{sc, PEO}=10) than the PPO side chains (N_{sc, PPO}=15). FIG. 5C shows PFG-NMR decay curves for B-E₅₄¹⁰P₈¹⁵. The polymer-only control and the polymer+liposome samples agree within experimental error, indicating that this polymer does not bind to a detectable extent.

FIG. 6A shows a comparison of the LDH release between linear and bottlebrush architectures and effect of molecular weight in the bottlebrush architecture. FIG. 6B shows the effect of shortening the PEO side chain on LDH release. FIG. 6C shows the effect of BBP architecture (statistical versus block copolymers) on LDH release. FIG. 6D shows the effect of bottlebrush PEO homopolymers and their molecular weight on LDH release. Error bars are the standard error of 9 independent replicates, and statistical significance indicates results from a one-way analysis of variance with Tukey's post hoc test (p<0.01).

FIG. 7 shows two possible non-limiting conformations of membrane-bound BBPs: flagpole and in-plane. R_{h, lipo}. is the hydrodynamic radius of liposomes extruded through a 100 nm diameter filter (this size was used only in the conformation assessment experiments, as described herein), L is an estimate of the distance the polymer protrudes normal to the liposome surface, A is an estimate of the surface area occupied per chain, and t is the bilayer thickness.

FIG. 8A shows intensity autocorrelation functions at each angle for multi-angle DLS data. The data were fit with the second cumulant model and the residuals were small. Shown are representative multi-angle DLS data confirming that liposome size distribution is unimodal with an Rₕ≈96.9 nm and a dispersity of 1.03. FIG. 8B shows the first cumulant as a function of the scattering vector. The linearity and near zero y-intercept indicate that the single relaxation process being observed is diffusive.

FIG. 9A and FIG. 9B show representative cryo-TEM micrographs from neat POPC liposomes. FIG. 9C and FIG. 9D show representative cryo-TEM micrographs from POPC liposomes treated with B-E₁₆₈⁴³P₄₃¹⁵ for 4 hours prior to vitrification.

FIG. 10 shows cloud point measurements of solutions of polymer in IM KF. From left to right: linear PEO (Mₙ=20 kDa), bottlebrush PEO (Mₙ=30.6 kDa), and a bottlebrush poloxamer (Mₙ=29.4 kDa).

FIG. 11A shows two hypothesized conformations for bound BBP: “flagpole” and “in-plane.” R_{h, lipo}. is the hydrodynamic radius of liposomes extruded through a 100 nm filter, L is an estimate of the distance the polymer protrudes normal to the liposome surface, A is an estimate of the surface area occupied per chain, and t is the bilayer thickness. FIG. 11B shows PFG-NMR decay curves over a series of liposome concentrations. Each dataset was fit to the sum of exponentials model and showed evidence of polymer binding to liposomes. FIG. 11C show estimated surface area coverage for both conformations based on the number of polymer chains bound to each liposome from the fits to the PFG-NMR data in FIG. 11B. Error estimated by propagating the standard deviation in f_{bound}. FIG. 11D shows DLS data for liposomes treated with B-E₁₁⁴³P₆¹⁵ under the same conditions as in FIG. 11B.

FIG. 12A shows multi-angle DLS data for POPC liposomes and POPC liposomes treated with B-E₁₆₈⁴³P₄₃¹⁵. FIG. 12B shows REPES analysis of the correlation function collected at 90°. A 100 nm diameter membrane was used to prepare the liposomes in these experiments.

FIG. 13A shows a sketch of how extending the backbone of the PEO block changes the molecular shape (between a star-like configuration and a bottlebrush-like configuration). FIG. 13B shows representative PFG-NMR decay curves for B-E₁₀⁴³P₅¹⁵ and liposomes treated with B-E₁₀⁴³P₅¹⁵. FIG. 13C shows representative PFG-NMR decay curves for B-E₅₇⁴³P₅¹⁵ and liposomes treated with B-E₅₇⁴³P₅¹⁵. FIG. 13D shows osmotic stress protection data over a series of polymer concentrations. FIG. 13E shows the effect of extending the PEO brush block on in vitro protection efficacy in mass units.

FIG. 14 shows PFG-NMR decay curves of a 1 mg/mL solution of B-E₁₁⁴³P₆¹⁵ in D₂O at two time points: 2 hours after dissolution (open circles) and 14 days after dissolution (open triangles). The decay curves and fittings to the bi-exponential model are within error, indicating that the same two populations are present: free chains and micelles. Additionally, when the 14-day decay curve was fit to the unconstrained tri-exponential model only two of the populations had non-zero coefficients, again indicating two populations: free chains and micelles.

FIG. 15 shows MALDI-TOF data of PPO polymer as synthesized via anionic polymerization (lower spectra, black) and post hydrogenation (upper spectra, gray). M_{w}=1220 g/mol and Ð=1.12 for both polymers. The most intense family of peaks in both samples is tert-butyl-PPO-OH (t-PPO-OH) and is unchanged during hydrogenation. Alkene and alkane α-chain end impurities are labelled accordingly.

FIG. 16A shows MALDI of t-PPO-OH and the NB functionalized macromonomer, t-PPO-NB, following the hydrogenation and esterification reactions. M_{w}=1210 g/mol and Ð=1.12 for the starting material (SM); and M_{w}=1330 g/mol and Ð=1.14 for the product (prod). FIG. 16B shows a ¹H NMR spectrum of the t-PPO-NB macromonomer. FIG. 16C shows a ¹H NMR spectrum of the purified methyl terminated PEO2k-NB macromonomer.

FIG. 17 shows SEC traces with differential refractive index detection of a

representative sequential ROMP polymer (BB-E_{45,20}-b-BB-P_{15,10}). The macromonomer traces are included for reference. From the multi-angle light scattering detector and a Zimm analysis, the BB-PPO aliquot has a M_{w}=11,600 g/mol and Ð=1.20; and the BB-diblock has a M_{w}=55,300 g/mol and Ð=1.10. Both were within error of the targeted molecular weight. The small peak at ˜17 minutes in the BB-PPO chromatogram is likely a small molecule impurity in the PPO macromonomer, possibly 18-crown-6 ether.

FIG. 18A shows excess scattering intensity at 37° C., as a function of polymer concentration. Linear PEO with an Mₙ=20 kDa is a negative control for micellization. FIG. 18B shows cryo-TEM micrograph of BB-E(45,160)-b-BB-P(15,43) at a concentration of 10 mg/mL in aqueous buffer with 40,000× magnification. FIG. 18C shows trend between CMCₐ and Mₙ for linear and bottlebrush poloxamers of similar wt % PEO. Data for linear poloxamers (40° C.) were taken from Alexandridis P et al., Macromolecules 1994;27(9):2414-2425. Data were fit to the exponential function shown on the graph and the correlation coefficients are 0.99 and 0.58 for linear and brush, respectively.

FIG. 19 shows the effect of the alkene α-endgroup impurity on the ROMP reaction. SEC with refractive index detection of a t-PPO-NB macromonomer and the bottlebrush polymer resulting from ROMP when the macromonomer was not hydrogenated. The dispersity is large and there is unreacted monomer, indicating that control over the polymerization is lost.

FIG. 20 shows a comparison of the SEC(dRI) traces of the t-PPO-OH prior to hydrogenation and the functionalized t-PPO-NB macromonomer post hydrogenation and esterification. The overlap of these chromatograms suggests that these reactions did not cause chain coupling or degradation.

FIG. 21A shows cryo-TEM micrographs of micelles of BB-E_{45,160}-b-BB-P_{15,43} at magnification of 40,000× and the same spot on the grid as FIG. 18B but with reduced exposure time. No beam damage was observable on the grid. FIG. 21B shows the same image as in FIG. 21A after the enhance local contrast filter has been applied. FIG. 21C shows 40,000× magnification on an additional spot on the grid. FIG. 21D shows 8,000× magnification of an additional spot on the grid.

FIG. 22A shows cryo-TEM micrographs of micelles of BB-E_{45,10}-b-BB-P_{15,5} at magnification of 20,000× and the same spot on the grid as FIG. 18B but with reduced exposure time. No beam damage was observable on the grid. FIG. 22B shows 20,000× magnification on an additional spot on the grid. FIG. 22C shows 8,000× magnification of an additional spot on the grid.

FIG. 23A shows excess scattering intensity of each polymer fit. FIG. 23B shows the relationship between Mₙ and CMCₐ, where the CMCₐ has been defined based on the model results.

FIG. 24A shows fitting of the electric field autocorrelation function to the second cumulant model (Eq. 7.1) of DLS correlation function fitting for a micelle solution of BB-E_{45,10}-b-BB-P_{15,5} at 10 mg/mL and 37° C. FIG. 24B shows residuals between the data and the fit for the second cumulant model. FIG. 24C shows fitting of the electric field autocorrelation function to a bi-exponential model (Eq. 7.2). FIG. 24D shows residuals between the data and the fit for the bi-exponential model.

FIG. 25A shows fitting of the electric field autocorrelation function to the second cumulant model (Eq. 7.1) of DLS correlation function fitting for a micelle solution of BB-E_{45,20}-b-BB-P_{15,10} at 10 mg/mL and 37° C. FIG. 25B shows residuals between the data and the fit for the second cumulant model. FIG. 25C shows fitting of the electric field autocorrelation function to a bi-exponential model (Eq. 7.2). FIG. 25D shows residuals between the data and the fit for the bi-exponential model.

FIG. 26A shows fitting of the electric field autocorrelation function to the second cumulant model (Eq. 7.1) of DLS correlation function fitting for a micelle solution of BB-E_{45,160}-b-BB-P_{15,43} at 10 mg/mL and 37° C. FIG. 26B shows residuals between the data and the fit for the second cumulant model. FIG. 26C shows fitting of the electric field autocorrelation function to a bi-exponential model (Eq. 7.2). FIG. 26D shows residuals between the data and the fit for the bi-exponential model.

FIG. 27A shows I′ found from the second cumulant model (Eq. 7.1) of multi-angle DLS data for a micelle solution of BB-E_{45,160}-b-BB-P_{15,43} at 3 mg/mL and 37° C. The slope gives a diffusion coefficient of 3×10⁻¹² m²/s. Based on the Stokes-Einstein equation, this corresponds to a hydrodynamic radius of Rₕ=110 nm. The near zero intercept and the high R² value suggests the relaxation process is diffusive. FIG. 27B shows I′₁ and I′₂ found from the bi-exponential model (Eq. 7.2). The slopes in this figure correspond to species with hydrodynamic radii of R_{h,1}=47 nm and R_{h,2}=160 nm. The linearity and high R² values suggest that both relaxation processes are diffusive.

FIG. 28A shows correlation function and analysis of a micellar solution of BB-E_{45,160}-b-BB-P_{15,43} at 10 mg/mL over a 35 day anneal at 37° C. After the day 1 measurement was performed, the sample was recovered and placed in a sealed scintillation vial on a hotplate for 35 days. FIG. 28B shows REPES analysis of the same correlation functions shown in FIG. 28A.

Both size distributions show a bimodal population, and the sizes of both populations are within error.

FIG. 29 shows a non-limiting schematic of a model used to estimate the micelle core dimensions from the polymer backbone and side chain degrees of polymerization. The model assumes that the backbone is a rigid rod, the side chains are Gaussian coils, and there is no chain overlap; thus, this model serves as an upper bound of micelle core dimensions. Note this is a two-dimensional projection, so the side chain crowding is exaggerated because in three dimensions, side chains will have more space to avoid one another.

FIG. 30 shows a non-limiting schematic of a linear polymer and various bottlebrush polymers including PEO (light gray) and PPO (dark gray) chains.

FIG. 31 shows non-limiting methods for in vitro live flexor digitorum brevis (FDB) skeletal muscle fiber contractility testing.

FIG. 32A-32C shows results from in vitro live FDB skeletal muscle fiber contractility testing with a non-limiting B-E(45,10)-P(15,5) bottlebrush polymer (Mₙ=26200 Da, D=M_{w}/Mₙ=1.07, 72 wt. % PEO). Provided are twitch contractile function of mdx-isolated, single live FDB skeletal muscle fibers before (FIG. 32A) and after (FIG. 32B) treatment with 1 nM of a bottlebrush polymer. Data show immediate enhancement of twitch contractile performance at very low dosing (1 nM) (FIG. 32C), which was at least 5000X more potent than a linear P188 polymer.

FIG. 33 shows non-limiting methods for in vivo cardiac stress testing.

FIG. 34 shows results from non-limiting live animal stress testing using mdx mice treated with bottlebrush (BB) copolymer or saline (as control). Black activity traces (5 mdx mice per group) indicate BB treated subjects. Gray traces (5 mdx mice per group) were saline controls. BB treatment included 0.15 mg/kg of copolymer by subcutaneous administration at 10 am on pretreatment day and on day of isoproterenol stress test (Iso) experiment. Iso treatment included 2 mg/ml isoproterenol provided at a dose of 10 mg/kg of body weight (or 5 μl/g of body weight) by intraperitoneal administration at 10 am, 2 pm, and 6 pm on day of the experiment. BB improves activity at more than 3000× lower dosing than a linear P188 polymer. In particular, BB-treated mice were protected after isoproterenol stress testing, whereas saline-treated mice were dying after stress testing.

### DETAILED DESCRIPTION

The present disclosure relates to bottlebrush polymers and methods of making and using such polymers.

The polymer can have any useful structure or configuration. In some cases, the polymer is a copolymer having two or more different side chains. In some cases, the copolymer can be a block copolymer or a statistical copolymer.

In some embodiments, the polymer has a number average molecular weight (Mₙ) of less than about 400 kDa, 300 kDa, 200 kDa, 100 kDa, or 60 kDa. In some embodiments, the Mₙ is from about 5 to 400 kDa or ranges therebetween (e.g., from 5 to 300 kDa, 5 to 200 kDa, 5 to 100 kDa, 5 to 60 kDa, 10 to 400 kDa, 10 to 300 kDa, 10 to 200 kDa, 10 to 100 kDa, 10 to 60 kDa, and the like).

In some cases, the polymer can include one or more polymeric side chains. In turn, the side chain can be a hydrophilic side chain or a hydrophobic side chain. In some embodiments, the hydrophilic side chain has increased solubility in water, as compared to the hydrophobic side chain.

Examples of non-limiting hydrophilic side chains include a heteroaliphatic group including one or more heteroatoms. In some cases, the hydrophilic side chain includes -Ak-X-, in which Ak is an optionally substituted alkylene and X is or includes a heteroatom, as defined herein (e.g., oxygen). In some cases, the hydrophilic side chain includes -[Ak-X]ₙ-, in which n is an integer from 1 to 50, Ak is an optionally substituted alkylene, and X is or includes a heteroatom (e.g., oxygen), as defined herein. In some cases, Ak is an unsubstituted alkylene, a linear alkylene, or an unsubstituted linear alkylene. In some cases, the hydrophilic side chain is ethylene oxide or a polymeric form thereof (e.g., poly(ethylene oxide) or PEO).

Examples of non-limiting hydrophobic side chains include a heteroaliphatic group including one or more heteroatoms, in which the heteroaliphatic group can be substituted with one or more substituents, as defined herein. In some cases, the hydrophobic side chain includes -Ak-X-, in which Ak is a substituted alkylene and X is or includes a heteroatom (e.g., oxygen), as defined herein. In some cases, the hydrophobic side chain includes -[Ak-X]ₙ-, in which n is an integer from 1 to 50, Ak is a substituted alkylene, and X is or includes a heteroatom (e.g., oxygen), as defined herein. In some cases, Ak is a substituted alkylene, a branched alkylene, or an optionally substituted branched alkylene. In some cases, the hydrophobic side chain is propylene oxide or a polymeric form thereof (e.g., poly(propylene oxide) or PPO). In particular cases, the alkylene can be substituted with one or more alkyl, halo, haloalkyl, alkyl-aryl, aryl-alkyl, or aryl groups.

In some embodiments, the number of carbons in Ak for the hydrophilic side chain is less than the number of carbons in Ak for the hydrophobic side chain. In some embodiments, Ak for the hydrophilic side chain is an optionally substituted C₁ alkylene, and Ak for the hydrophobic side chain is an optionally substituted C₂₋₆ alkylene (e.g., an optionally substituted C₂₋₆ linear or branched alkylene). In some embodiments, Ak for the hydrophilic side chain is an optionally substituted C₁₋₂ alkylene, and Ak for the hydrophobic side chain is an optionally substituted C₃₋₆ alkylene (e.g., an optionally substituted C₃₋₆ linear or branched alkylene). In some embodiments, Ak for the hydrophilic side chain is an optionally substituted C₁₋₃ alkylene, and Ak for the hydrophobic side chain is an optionally substituted C₄₋₆ alkylene (e.g., an optionally substituted C₄₋₆ linear or branched alkylene). In some embodiments, Ak for the hydrophilic side chain is an optionally substituted C₁₋₄ alkylene, and Ak for the hydrophobic side chain is an optionally substituted C₅₋₈ alkylene (e.g., an optionally substituted C₅₋₈ linear or branched alkylene).

In any case herein, X in a side chain can include a heteroatom or a functional group that includes a heteroatom. For instance and without limitation, X can be oxy (i.e., —O—). In another instance, X can include an oxygen atom, such as in, e.g., C(═O)—O—, —O—C(═O)—, or —C(═O)—.

In any case herein, Ak in a side chain can include a substitution that includes a heteroatom or a functional group that includes a heteroatom. For instance and without limitation, Ak can be substituted to include an oxo group (i.e., (═O)) on the alkylene chain.

In some cases, the side chain can include a polyether, a polyester, and the like. Non-limiting examples of polyethers include poly(ethylene glycol) (PEG), poly(propylene glycol) (PPG), poly(oxymethylene) (POM), poly(tetramethylene glycol) (PTMG), poly(ethyl ethylene) phosphate (PEEP), and poly(oxazoline). Non-limiting examples of polyesters include poly(glycolic acid) (PGA), poly(lactic acid) (PLA) including poly(L-lactide) (PLLA) and poly(D-lactide) (PDLA), poly(lactic-co-glycolic acid) (PLGA), poly(caprolactone) (PCL), poly(lactide-co-caprolactone), poly(butyric acid), poly(valeric acid), polyhydroxyalkanoate (PHA), polyhydroxybutryate (PHB), polyethylene adipate (PEA), polybutylene succinate (PBS), and poly(3-hydroxybutyrate-co-3-hydroxyvalerate) (PHBV).

The backbone can include any type and number of repeating units. In some cases, the backbone can include a norbornene derivative, such as any described herein.

The backbone and the side chains can be attached in any useful way. In some cases, the repeating unit of the backbone is linked to the side chain by way of a linker, such as, e.g., C(═O)—O—, —O—C(═O)—, —C(═O)—, a covalent bond, an aliphatic group (e.g., alkylene), or an heteroaliphatic group (e.g., heteroalkylene).

The compositions and compounds herein can be used alone or in combination with one or more agents. Such agents can include a pharmaceutically acceptable excipient, a solvent (e.g., an aqueous solvent, an organic solvent, or a mixture thereof), a therapeutic agent (e.g., a drug), or combinations thereof. Non-limiting examples of therapeutic agents include a protein (e.g., an antibody), a peptide, a nucleic acid (e.g., a DNA, RNA, mRNA, or combinations thereof), a small molecule, a cytokine, a growth factor, a drug (e.g., an antibiotic, an antiviral, an anti-proliferative agent, an anti-cancer agent, a non-steroidal anti-inflammatory drug, a steroid, and the like), an ionic agent, a diagnostic agent, a lipid, a lipoprotein, a hormone, a vitamin, a carbohydrate, a saccharide, a label, a fluorophore, a dye, or a combination of any of these. Other therapeutic agents can include any described herein.

The present disclosure also encompasses methods for using and making any composition or compound described herein. In some cases, the method includes stabilizing an interface (e.g., between two different phases) by providing a composition or compound described herein. The interface can be present between any two phases, such as a liquid-liquid phase. In some cases, the interface can further include a lipid, a surfactant, or an amphiphile disposed between the two phases. In some cases, the interface can be disposed on or near a lipid monolayer, a lipid bilayer, a lipid multilayer, a micelle, a vesicle, a lipid particle, or a lipid complex.

In some cases, the method includes stabilizing a cell by providing a composition or compound described herein. The extent of stability can be determined in any useful manner, e.g., by measuring osmotic stress, as described herein. The cell can be provided as a single cell or a plurality of cells. Furthermore, the cell can be provided in a suspension, a culture, or a tissue.

In some cases, the method includes treating a disease, a disorder, or a condition by providing a composition or compound described herein. Non-limiting examples of diseases, disorders, and conditions include skeletal muscle disorder, sickle cell disease, reperfusion disease or reperfusion injury, ischemia, and cardiovascular disease, as well as others described herein.

In some cases, the method includes preparing a monomer (e.g., for use in providing any polymer herein) or a polymer. The method can include any useful combination of processes and reactions to provide side chains configured to be grafted onto monomers or polymers, to provide monomers (including macromonomers) for use in polymerization, and to provide a bottlebrush polymer.

In some cases, the method can include preparing a monomer or a compound herein. As used herein, a monomer refers to any compound that can be further reacted to provide a polymer. In one instance, the monomer can provide a polymerized side chain. In another instance, a monomer can provide a polymerized backbone. In yet another instance, a monomer can provide a bottlebrush polymer having polymerized side chains and a polymerized backbone.

In particular cases, a monomer can be prepared by way of polymerization (e.g., to provide a polymerized side chain). Polymerization can be performed in any useful manner. In one case, polymerization can include anionic polymerization in the presence of a first monomer (e.g., an epoxide-containing monomer), thereby providing a first compound having a side chain. In some cases, the side chain can include a polymeric side chain. A non-limiting example of a first monomer includes

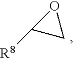

wherein R⁸ is hydrogen, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl. Non-limiting examples of first compounds having side chains include a compound of Formula IIIA or IIIB, wherein q is any useful integer (e.g., from 10-20), R⁷′ does not include alkenyl, R⁷″ comprises alkenyl, and each of R⁷′ and R⁷″ is a reacted form of R^{7a}.

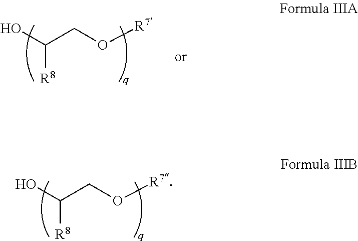

In some cases, a compound (e.g., a first compound having a side chain) can be hydrogenated, thereby providing a second compound having saturated groups (e.g., saturated, polymerized side chains). In some cases, the second compound can lack unsaturated groups (e.g., lack alkenyl groups, alkynyl groups, or both alkenyl and alkynyl groups). A non-limiting example of a second compound includes a compound of Formula IIIC, wherein R⁷″* comprises an unsaturated version of R⁷:

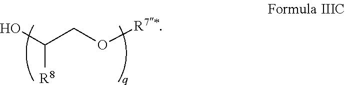

Hydrogenation can be used to control the extent of saturation in a compound. In one case, hydrogenation is used with any initial compound to provide a saturated side chain. Any useful hydrogenation catalyst can be employed, such as palladium (e.g., on carbon), platinum, rhodium, ruthenium, nickel (e.g., Raney nickel), and the like. Heterogeneous and homogeneous catalysts may be employed.

In some cases, a compound having a side chain can be reacted with a backbone monomer that provides repeating units for the backbone of the polymer. Upon reaction, the resulting macromonomer can include a side chain portion and a backbone portion. For instance and without limitation, such reactions can include esterification, nucleophilic substitution, and the like, thereby providing the macromonomer. A non-limiting example of a macromonomer includes Formula III, III-1, III-2, or III-3, wherein R⁴, R⁵, R⁶, R⁷, R⁸, n, and q can be any described herein:

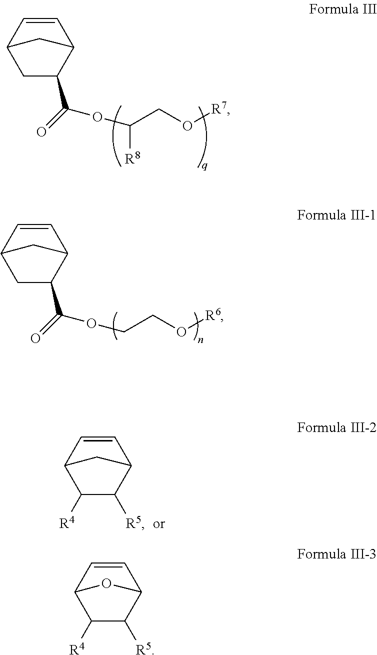

Any useful backbone monomer can be used. In some non-limiting cases, the backbone monomer can include a norbornene derivative that optionally includes one or more functional groups. Non-limiting examples of functional groups include a carboxyl moiety (—CO₂H), an acyl halide moiety (—COX, in which X is halo), an ester moiety (e.g., —CO₂R, in which R is optionally substituted aliphatic or optionally substituted aromatic), an anhydride moiety (e.g., —C(O)—O—C(O)—), an amido moiety (e.g., —C(O)NR^{N1}R^{N2}, in which each R^{N1} and R^{N2} is, independently, H, optionally substituted aliphatic, or optionally substituted aromatic), a dicarboximide moiety (e.g., —C(O)—NR^{N1}—C(O)—, in which R^{N1} is H, optionally substituted aliphatic, or optionally substituted aromatic), and the like. Non-limiting norbornene derivatives include norbornene, 2,5-norbornadiene, 7-oxanorbornene, bicyclo[2.2.2]oct-2-ene, and the like.

Polymerization can be used to prepare a polymer with one or more monomers (e.g., any monomer described herein, including macromonomers). Furthermore, sequential polymerization can be employed to prepare an initial polymer by reacting an initial monomer with a polymerization catalyst, and then to react the initial polymer with a further monomer to provide a further polymer (e.g., a copolymer) having both initial monomers and further monomers.

Polymerization can be conducted in any useful manner. In some cases, a polymerization catalyst (e.g., a Grubbs catalyst) can be employed. Non-limiting polymerization reactions can include ring-opening metathesis polymerization (ROMP), cationic ring-opening polymerization (CROP), anionic ring-opening polymerizations (AROP), isomerization polymerization, addition polymerization, reversible addition-fragmentation chain transfer (RAFT), atom transfer radical polymerization (ATRP), and the like. Non-limiting polymerization catalysts include those having one or more transition metals (e.g., ruthenium (Ru), molybdenum (Mo), rhenium (Re), tungsten (W), titanium (Ti), and the like). Yet other non-limiting polymerization catalysts include Grubbs catalysts, including first-generation Grubbs catalyst, second-generation Grubbs catalysts, third-generation Grubbs catalysts, or Hoveyda-Grubbs catalysts. Further non-limiting polymerization catalysts include dichloro[1,3-bis(2,4,6-trimethylphenyl)-2-imidazolidinylidene](benzylidene)bis(3-bromopyridine)ruthenium(II) (available as Grubbs Catalyst® M300 from Sigma-Aldrich, St. Louis, MO), dichloro (benzylidene)bis(tricyclohexylphosphine)ruthenium(II) (available as Grubbs Catalyst® M102 from Sigma-Aldrich), dichloro[1,3-bis(2,4,6-trimethylphenyl)-2-imidazolidinylidene] (benzylidene)(tricyclohexylphosphine)ruthenium(II) (available as Grubbs Catalyst® M204 from Sigma-Aldrich), dichloro[1,3-bis(2-methylphenyl)-2-imidazolidinylidene](benzylidene) (tricyclohexylphosphine)ruthenium(II) (available as Grubbs Catalyst® M205 from Sigma-Aldrich), dichloro[1,3-bis(2,4,6-trimethylphenyl)-2-imidazolidinylidene](3-methyl-2-butenylidene) (tricyclohexylphosphine)ruthenium(II) (available as Grubbs Catalyst® M207 from Sigma-Aldrich), dichloro[1,3-bis(2,4,6-trimethylphenyl)-2-imidazolidinylidene][3-(2-pyridinyl) propylidene]ruthenium(II) (available as Grubbs Catalyst® M360 from Sigma-Aldrich), dichloro[1,3-bis(2,4,6-trimethylphenyl)-2-imidazolidinylidene](2-isopropoxyphenylmethylene) ruthenium(II) (available as Hoveyda-Grubbs Catalyst® M720 from Sigma-Aldrich), dichloro(2-isopropoxyphenylmethylene) (tricyclohexylphosphine)ruthenium(II) (available as Hoveyda-Grubbs Catalyst® M700 from Sigma-Aldrich), and dichloro[1,3-bis(2-methylphenyl)-2-imidazolidinylidene](2-isopropoxyphenylmethylene)ruthenium(II) (available as Hoveyda-Grubbs Catalyst® M721 from Sigma-Aldrich).

Polymerization can include any useful monomer. In some cases, polymerization can be conducted in the presence of an initial monomer having a first polymerized side chain. In some cases, polymerization can be conducted in the presence of an initial monomer having a first polymerized side chain and a further monomer having a second polymerized side chain. The sequence of adding such monomers can be conducted in any useful manner that provides desired segments, blocks, or arrangement of the monomers in the polymer.

In some cases, polymerization can include the addition of a diluent, which can be used to control the composition and structure of the polymer. For example and without limitation, a diluent can be included to control the graft density or graft location of the side chains within the polymer. In use, a diluent can be any monomer that reacts with a backbone portion of another monomer. The diluent can have any useful structure. In some cases, the diluent has a structure of Formula IVA or IVB, as well as isomers thereof:

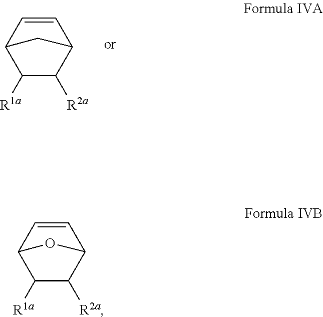

wherein each of R¹a and R²a is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R¹a and R^{2a}, when taken together, form an anhydride moiety or a dicarboximide moiety. Isomers can include stereoisomers, such as enantiomers or diastereomers thereof. Yet other examples of diluents include the following:

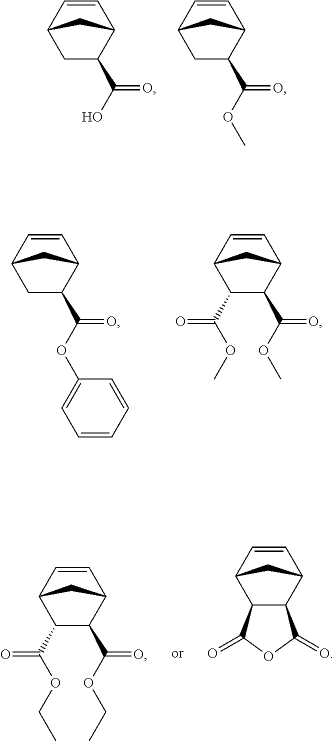

The polymer can have any useful number of repeating units in the backbone that are formed by same or different monomers. In some cases, the polymer can have an m or p or r number of repeating units within the backbone. Non-limiting examples of m, p, and/or r includes more than about 2, 5, 10, 20, 30, 40, 50, 60, 70, 80, 90, 100, 120, 140, 160, 180, 200, 300, 400 or more or from about 2 to 400 (e.g., from 2 to 10, 2 to 20, 2 to 30, 2 to 40, 2 to 50, 2 to 60, 2 to 70, 2 to 80, 2 to 90, 2 to 100, 2 to 120, 2 to 140, 2 to 160, 2 to 180, 2 to 200, 2 to 300, 3 to 10, 3 to 20, 3 to 30, 3 to 40, 3 to 50, 3 to 60, 3 to 70, 3 to 80, 3 to 90, 3 to 100, 3 to 120, 3 to 140, 3 to 160, 3 to 180, 3 to 200, 3 to 300, 3 to 400, 5 to 10, 5 to 20, 5 to 30, 5 to 40, 5 to 50, 5 to 60, 5 to 70, 5 to 80, 5 to 90, 5 to 100, 5 to 120, 5 to 140, 5 to 160, 5 to 180, 5 to 200, 5 to 300, 5 to 400, 8 to 20, 8 to 30, 8 to 40, 8 to 50, 8 to 60, 8 to 70, 8 to 80, 8 to 90, 8 to 100, 8 to 120, 8 to 140, 8 to 160, 8 to 180, 8 to 200, 8 to 300, 8 to 400, 10 to 20, 10 to 30, 10 to 40, 10 to 50, 10 to 60, 10 to 70, 10 to 80, 10 to 90, 10 to 100, 10 to 120, 10 to 140, 10 to 160, 10 to 180, 10 to 200, 10 to 300, 10 to 400, or any ranges therebetween). In some cases, m is 0. In some cases, p is 0. In some cases, r is 0.

The polymer can have any useful number of repeating units within a side chain. In some cases, the polymer can have an n or q number of repeating units within the side chain. Non-limiting examples of n and/or q includes more than about 2, 5, 10, 20, 30, 40, 50, 60, 70, 80, 90, 100, 120, 140, 160, 180, 200, or more or from about 2 to 200 (e.g., from 2 to 10, 2 to 20, 2 to 30, 2 to 40, 2 to 50, 2 to 60, 2 to 70, 2 to 80, 2 to 90, 2 to 100, 2 to 120, 2 to 140, 2 to 160, 2 to 180, 3 to 10, 3 to 20, 3 to 30, 3 to 40, 3 to 50, 3 to 60, 3 to 70, 3 to 80, 3 to 90, 3 to 100, 3 to 120, 3 to 140, 3 to 160, 3 to 180, 3 to 200, 5 to 10, 5 to 20, 5 to 30, 5 to 40, 5 to 50, 5 to 60, 5 to 70, 5 to 80, 5 to 90, 5 to 100, 5 to 120, 5 to 140, 5 to 160, 5 to 180, 5 to 200, 8 to 20, 8 to 30, 8 to 40, 8 to 50, 8 to 60, 8 to 70, 8 to 80, 8 to 90, 8 to 100, 8 to 120, 8 to 140, 8 to 160, 8 to 180, 8 to 200, 10 to 20, 10 to 30, 10 to 40, 10 to 50, 10 to 60, 10 to 70, 10 to 80, 10 to 90, 10 to 100, 10 to 120, 10 to 140, 10 to 160, 10 to 180, 10 to 200, or any ranges therebetween).

In some cases, the polymer comprises a structure of Formula IA:

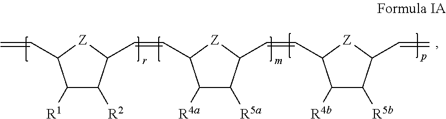

wherein:
Z is oxy (—O—) or C₁₋₆ alkylene (e.g., methylene);
each of R¹ and R² is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R¹ and R², when taken together, form an anhydride moiety or a dicarboximide moiety;
each of R⁴a and R⁴b is, independently, hydrogen or C₁₋₆ alkyl;
each of R⁵a and R⁵b is, independently, the hydrophilic side chain or the hydrophobic side chain;
m is 10-200; p is 0-50; and r is 0-180.

In some cases (e.g., of Formula IA), Z is methylene, R⁵a is a hydrophilic side chain, and R⁵b is a hydrophobic side chain. In some cases, p is 5-50. In some cases, r is 1-180. In some cases, R⁵a is a hydrophilic polymeric side chain. In some cases, R⁵b is a hydrophobic polymeric side chain.

In some cases, the polymer comprises a structure of Formula IB:

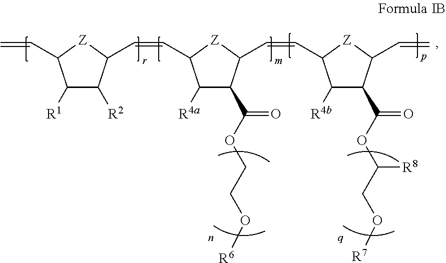

wherein:
Z is oxy (—O—) or C₁₋₆ alkylene (e.g., methylene);
each of R¹ and R² is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R¹a and R^{2a}, when taken together, form an anhydride moiety or a dicarboximide moiety;
each of R⁴a and R⁴b is, independently, hydrogen or C₁₋₆ alkyl;
each R⁶ is, independently, hydrogen or C₁₋₆ alkyl;
each R⁷ is, independently, hydrogen, C₁₋₁₂ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
each R⁸ is, independently, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
m is 10-200; n is 5-200; p is 0-50; q is 5-20; and r is 0-180.

In some cases, the polymer comprises a structure of Formula IC:

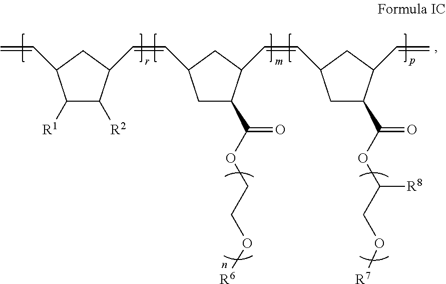

wherein R¹, R², R⁶, R⁷, R⁸, m, n, p, q, and r can be any described herein.

In some cases, the polymer comprises a structure of Formula II:

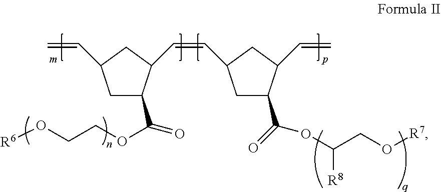

wherein R⁶, R⁷, R⁸, m, n, p, and q can be any described herein.

For any compounds described herein (e.g., monomers, polymers, or other compounds), one or more asymmetric centers may be present. Accordingly, any compounds or compositions herein can include stereoisomeric forms thereof, e.g., enantiomers thereof, diastereomers thereof, as well as mixtures including two or more different stereoisomeric forms.

For any compounds described herein (e.g., monomers, polymers, or other compounds), one or more leaving groups may be present. Such leaving groups can include an atom or a group capable of being displaced by a nucleophile and, as such, allow for reactions to occur between compounds or between functional groups. Non-limiting leaving groups include halo, hydroxyl, alkoxy, sulfonate, nitrate, phosphate, carboxylate, or carbonyl moieties, as well as substituted forms having an optionally substituted aliphatic group or an optionally substituted aromatic group.

### Methods of Use

The methods, compounds, and compositions herein can be used to treat a disease, a disorder, a condition, or a physiological state. As described herein, the polymers herein, as well as compositions including such polymers, can be used to stabilize an interface (e.g., a hydrophobic interface). In some embodiments, the interface can be present on a surface of a biological component or within a biological component. Non-limiting examples of biological components include one or more lipids, membranes, cells (e.g., cardiomyocytes, skeletal muscle cells, neuronal cells, and the like), cellular fragments, tissues (e.g., muscle, cartilage, and the like), biological fluids (e.g., such as blood, plasma, interstitial fluid, etc.), organs, and the like.

Without wishing to be limited by mechanism, providing stability at such interfaces can result in repairing damage at such interfaces. In turn, such damage can result from any number of various diseases, disorders, conditions, or physiological states (e.g., radiation exposure, electric shock, free radical injury, ischemia/reperfusion injuries occurring after heart attacks and strokes, Duchenne's muscular dystrophy, organ preservation conditions, organ transplant conditions, oxidative stress, and others). Such damage can include reduced permeability at the interfaces, decreased cellular function, increased apoptosis or cell death, and the like; and use of at least one polymer (e.g., any described herein) can be used to potentially treat or repair such damage.

In some embodiments, one or more diseases, disorders, conditions, or physiological states can include, but are not limited to, the following: skeletal muscle disorder (e.g., muscular dystrophy, Duchenne's muscular dystrophy, skeletal muscle injury, dystrophin-deficient skeletal muscle, skeletal muscle having a contraction force deficit, skeletal muscle having a Ca2+ imbalance, skeletal muscle having microtears, and the like); sickle cell disease (e.g., sickle cell anemia, sickle cell trait, thalassemia, and the like); reperfusion disease or reperfusion injury; ischemia (e.g., myocardial infarction, limb ischemia, and the like); cardiovascular disease (e.g., diastolic dysfunction, heart failure, ischemic heart failure, congestive heart failure, myocardial infarction, cardiomyopathy, arrythmia, fibrillation, angina, peripheral artery disease, peripheral vascular disease, and the like); shock (e.g., septic shock, hemorrhagic shock, and the like); stroke (e.g., hemorrhagic shock, ischemic stroke, acute stroke, and the like); blood loss (e.g., acute blood loss, blood loss due to trauma or surgery); blood disorder (e.g., anemia, and the like); neurodegenerative disease (e.g., Alzheimer's disease); macular degeneration, diabetic retinopathy; respiratory disease (e.g., acute respiratory distress syndrome (ARDS)); multiple organ failure; acidosis; hypothermia; cartilage damage; osteoarthritis; chronic microvascular disease (e.g., macular degeneration, diabetic retinopathy, and congestive heart failure); wound healing; and the like.

In some embodiments, one or more diseases, disorders, conditions, or physiological states can include, but are not limited to, the following: treating disorders treated by membrane resealing and repair; treating tissue ischemia and reperfusion injury; reducing inflammatory responses; reducing blood viscosity; facilitating thrombolysis; promoting or maintaining hemostasis; as a vehicle for drug, nucleic acid or protein delivery; as an emulsifier to stabilize suspensions of hydrophobic drugs; cleansing skin wounds; as a surfactant in the formulation of cosmetics; as a component for use during organ transplant, tissue preservation, cryopreservation, or organ preservation; to treat storage lesion compromised blood or prevent storage lesion in blood and blood products, to control the viscosity of personal care products and soaps; as a laxative and other uses known to those of skill in the art.

Any composition herein can be formulated in any useful manner. In some embodiments, a pharmaceutical composition can include at least one (e.g., two, three, or more) polymer including a pharmaceutically acceptable excipient (e.g., any described herein) and/or a pharmaceutically acceptable carrier (e.g., any described herein). In some embodiments, the polymer can be pre-treated, sterilized, lyophilized, dried, dissolved, suspended, dispersed, mixed, diluted, and the like to provide the pharmaceutical composition.

In some embodiments, the pharmaceutical composition can further include at least one (e.g., two, three, or more) therapeutic agent. Non-limiting examples of therapeutic agents include a diuretic, a loop diuretic, a potassium sparing agent, a vasodilator, an ACE inhibitor, an angiotensin II antagonist, a positive inotropic agent, a phosphodiesterase inhibitor, a beta-adrenergic receptor antagonist, a calcium channel blocker, a nitrate, an alpha blocker, a central alpha antagonist, a statin, or a combination of these agents. Yet other non-limiting examples of therapeutic agents include streptomycin, corticosteroids (e.g., prednisone, deflazacort), immunosupressive agents (e.g., azathioprine, cyclosporine), valproic acid, phenylbutyrate, sodium butyrate, M344 (a benzamide and histone deacetylase [HDAC] inhibitor; 4-(dimethylamino)-n-[7-(hydroxyamino)-7-oxoheptyl]benzamide), suberoylanilide hydroxamic acid (SAHA), ataluren (PTC124; 3-[5-(2-fluorophenyl)-1,2,4-oxadiazol-3-yl]benzoic acid), or a combination of any of these.

Pharmaceutical compositions can include any useful formulation, including but not limited to solutions, suspensions, emulsions, and other such mixtures, and can be formulated as non-aqueous or aqueous mixtures, creams, gels, ointments, emulsions, solutions, elixirs, lotions, suspensions, tinctures, pastes, foams, aerosols, irrigations, sprays, injections, suppositories, bandages, implants, explants, cartridges, or any other formulation suitable for systemic, topical, or local administration.

Selection of the formulation, carrier and/or excipient is within the skill of the administering professional and can depend upon a number of parameters. These include, for example, the mode of administration (e.g., systemic, intravenous, subcutaneous, intramuscular, intradermal, parenteral, intraarticular, intracisternal, intraocular, intraarterial, intraventricular, intrathecal, intraperitoneal, intratracheal, oral, nasal, pulmonary, local, topical, or any other mode) and the disease, disorder, or condition to be treated. Administration can include, but are not limited to, the following: coronary or cardiac reperfusion, transfusion, intravenous infusion, and the like. In some embodiments, administration of the polymer can include one or more other compounds, such as blood, red blood cells and/or blood products, such as packed red blood cells.

Dosages and regimens can be determined by one skilled in the art. In some embodiments, the at least one polymer can be formulated at a concentration ranging from at least about 1 μg/mL (e.g., at least about 2 μg/mL, at least about 10 μg/mL, at least about 50 μg/mL, at least about 100 μg/mL, at least about 500 μg/mL, at least about 1 mg/mL, at least about 10 mg/mL, at least about 50 mg/mL, or at least about 100 mg/mL) to at most about 300 mg/ml (e.g., at most about 100 mg/mL, at most about 50 mg/mL at most about 10 mg/mL, at most about 1 mg/mL, at most about 500 μg/mL, at most about 100 μg/mL, at most about 50 μg/mL, at most about 10 μg/mL, or at most about 5 μg/mL) for administration. In some embodiments, the at least one polymer can be formulated at a concentration ranging at most about 300 mg/mL, at most about 100 mg/mL, at most about 50 mg/mL at most about 10 mg/mL, at most about 1 mg/mL, at most about 500 μg/mL, at most about 100 μg/mL, at most about 50 μg/mL, at most about 10 μg/mL, at most about 5 μg/mL, or at most about 1 μg/mL for administration.

In some embodiments, the at least one polymer can be formulated for administration to a subject at a dosage of about 0.01 μg/kg of body weight (e.g., at least about 0.05 μg/kg, at least about 0.1 μg/kg, at least about 0.5 μg/kg, at least about 1 μg/kg, at least about 10 μg/kg, at least about 0.02 mg/kg, at least about 0.05 mg/kg, at least about 0.1 mg/kg, at least about 0.2 mg/kg, at least about 0.5 mg/kg, at least about 1 mg/kg, at least about 2 mg/kg, at least about 5 mg/kg, at least about 10 mg/kg, at least about 50 mg/kg, at least about 100 mg/kg, at least about 200 mg/kg, at least about 300 mg/kg, at least about 400 mg/kg, at least about 500 mg/kg, or at least about 1000 mg/kg) to about 2000 mg/kg of body weight (e.g., at most about 1000 mg/kg, at most about 500 mg/kg, at most about 100 mg/kg, at most about 10 mg/kg, at most about 5 mg/kg, at most about 1 mg/kg, at most about 0.5 mg/kg, at most about 0.1 mg/kg, at most about 0.05 mg/kg, at most about 0.01 mg/kg, at most about 1 μg/kg. or at most about 0.1 μg/kg). The dosage may depend upon the disease, disorder, or condition to be treated.

The present disclosure will be further described in the following examples, which do not limit the scope described in the claims.

### EXAMPLES

### Example 1

### Effect of Bottlebrush Poloxamer Architecture on Binding to Liposomes

Many pathologies can be linked to cell membrane damage, such as radiation exposure, electric shock, ischemia/reperfusion injuries occurring after heart attacks and strokes, and Duchenne's muscular dystrophy. This damage can prevent the cell from regulating transport of nutrients and waste and from maintaining a transmembrane ion gradient, resulting in cell and tissue death. Poloxamers, a family of commercially available block polymer amphiphiles consisting of poly(ethylene oxide) (PEO) and poly(propylene oxide) (PPO) blocks, have demonstrated in vitro efficacy in protecting cardiomyocyte and neuronal cells from oxidative stress, reduced permeability of damaged blood brain barriers, proven in vivo efficacy in mouse and pig ischemia/reperfusion models, and enabled heart and skeletal muscle function of dystrophic mice to resemble that of healthy mice.

Simplified abiotic systems including lipid monolayers, bilayers, or liposomes of various lipid compositions can be used to explore the mechanism(s) of membrane stabilization. Experiments on lipid monolayers have revealed that poloxamers can insert into damaged regions due to the reduced surface pressure. Despite such studies of polymer-membrane interactions, a complete understanding of the stabilization mechanism is lacking, hindering the engineering of more effective therapeutics.

In some non-limiting instances, bottlebrush polymers can include polymeric side chains that are densely grafted onto a central backbone, characterized by the backbone degree of polymerization (N_{bb}), the side chain degree of polymerization (N_{sc}), and the average degree of polymerization between side chains (N_{g}). Compared to linear polymers, the steric repulsions among adjacent side chains can lead to larger persistence lengths and reduced entanglement densities. Polymer properties and interactions with other macromolecules can be tuned by manipulating bottlebrush architectural parameters. For example, the segmental relaxation time (chain mobility) can be a function of side chain length. Longer side chains can decrease steric constraints at the units far from the backbone, which enhances interdigitation between chains. Therefore, by combining an efficient strategy to synthesize bottlebrush polymers with PEO and PPO side chains—denoted bottlebrush poloxamers (BBPs)—with knowledge of parameter-property relationships, the bottlebrush architecture can be used as a tool to inform the stabilization mechanism(s) of block polymers.

Herein, described are comparisons between linear poloxamer and an analogous BBP, in which the BBP generally exhibits a higher membrane affinity but similar in vitro protection efficacy. To better understand the effect of bottlebrush architectural parameters on membrane affinity, systematic molecular variations were performed, and a pulsed-field-gradient NMR (PFG-NMR) binding assay was applied to elucidate the roles of the hydrophobic backbone, overall molecular weight, side chain length asymmetry, and statistical versus block architectures. Such studies can be used to inform engineering of more effective membrane binders and elucidate mechanisms of polymer-lipid bilayer interactions.

### Example 2

### Preparation of Materials

The following materials were purchased from Sigma Aldrich and used without further purification: propylene oxide (>99.0%), 18-crown-6 ether, tetrahydrofuran (ACS reagent, 97%, stabilized with 250 ppm butylated hydroxytoluene), butyl-magnesium chloride, potassium tert-butoxide, poly(ethylene oxide) monomethyl ether (Mₙ˜2000 g/mol), poly(ethylene oxide) monomethyl ether (Mₙ˜350 g/mol), N,N′ diisopropylcarbodiimide (99%), 4-dimethylaminopyridine (>99%), exo-5-norbornene-2-carboxylic acid (97%), α-cyano-4-hydroxycinnamic acid, sodium trifluoroacetate, Grubbs second generation catalyst M204, pyridine (>99%), ethyl vinyl ether (stabilized with 0.1% KOH, 99%), benzene (ACS reagent, 99%), sodium chloride (99.5%), magnesium chloride (97%), calcium chloride (anhydrous, 96%), potassium chloride (>99%), HEPES buffer (99.5%), and Triton X-100. The following materials were purchased from Fisher Scientific and used as received: methanol (HPLC grade), dichloromethane (DCM; ACS reagent, anhydrous, 99.8%), diethyl ether (anhydrous), chloroform, and diatomaceous earth. NMR solvents deuterium oxide and chloroform-d (99.8 atom % d) were purchased from Cambridge Isotope Laboratories. 16:0-18:1 1-palmitoyl-2-oleoyl-glycero-3-phosphocholine (POPC) was purchased from Avanti Polar Lipids and used as received. A lactate dehydrogenase (LDH) assay kit was purchased from Pointe Scientific Inc. SiliaMetS® DMT was obtained from Silicycle.

Synthesis and characterization of BBPs: Synthesis methods were described in detail in a Hassler J F et al., ACS Macro Lett. 2022; 11:460-467. Briefly, norbornene-functionalized macromonomers (MMs) were synthesized by chain-end modification of mono-alcohol functionalized poly(propylene oxide) (PPO) and poly(ethylene oxide) (PEO). The PPO polymers were prepared via anionic polymerization. The PEO polymers were purchased and freeze dried from benzene. Three MMs were synthesized: PPO with Mₙ=1090 g/mol (PPO-1k), PEO with Mₙ=2060 g/mol (PEO-2k), and PEO with Mₙ=600 g/mol (PEO-0.6k). To install the norbornene functional group, a condensation reaction was performed between 1 equivalent (equiv.) of mono-alcohol functionalized polymer and 1.5 equiv. of exo-5-norbornene-2-carboxylic acid in the presence of 0.2 equiv. of 4-dimethylaminopyridine and 1.5 equiv. of N,N′-diisopropylcarbodiimide at room temperature in anhydrous DCM at a polymer concentration of 0.05 M. Condensation reactions of PEO MMs were run for 48 hours (h) while the PPO MM required 7 days (d). Note that in the case of PPO, a hydrogenation reaction was performed prior to the condensation reaction to eliminate alkene α-chain end impurities. Percent norbornene functionality was assessed via matrix-assisted laser desorption/ionization time of flight (MALDI-ToF) spectrometry and 1H NMR spectroscopy and was found to be quantitative for PPO-1k, 94% for PEO-2k, and 92% for PEO-0.6k. PEO-2k was purified via two precipitations in cold diethyl ether and PPO-1k and PEO-0.6k were purified by vacuum drying at 40° C. for 7 d.

After macromonomer purification, two sequential ring-opening metathesis polymerization reactions were performed in a glovebox with an argon atmosphere to yield diblock BBPs. An example protocol is: 1 equiv. of Grubbs third generation catalyst (prepared by methods reported by Love J A et al., Angew. Chemie—Int. Ed. 2002;41(21):4035-4037) was added to the reaction vessel containing the PPO-1k MM dissolved in anhydrous DCM at 0.05 M. The reaction was stirred for 10 minutes (min), and then the second MM was added also at 0.05 M. After 10 min, the reaction was removed from the glovebox and was diluted by half with a 1:1 (v:v) mixture of ethyl vinyl ether: DCM to quench the reaction. This mixture was then stirred over SiliaMetS® DMT for a minimum of 3 h to chelate the deactivated catalyst, which was then removed by filtering through a column of Celite® (purchased from Fisher Scientific). The solvent was removed via rotary evaporation, and the product was freeze-dried from benzene. For statistical copolymers, two macromonomers were mixed in the desired stoichiometric ratios and then the catalyst was added.

The conversion and composition of the resulting polymers were characterized by ¹H NMR spectroscopy on a Bruker Avance III HD-500 MHz spectrometer equipped with a 5 mm Prodigy TCI cryoprobe. The molecular weight distributions were characterized by size exclusion chromatography equipped with a Wyatt Dawn Heleos II multi-angle light scattering detector. The refractive index increment (dn/dc) of the BBPs was taken as the mass fraction weighted average of the dn/dc values of PEO (0.068 mL/g) and PPO (0.087 mL/g) in tetrahydrofuran.

Liposome production: POPC powder, typically ˜15 mg, was dissolved in a minimal amount of chloroform. The chloroform was then evaporated using a gentle, filtered stream of nitrogen (0.4 μm PTFE filter) to yield a thin film of lipid around the vial walls. This film was then hydrated with 1 mL of D₂O and placed on a vortex plate for 1 h. The resulting disperse mixture of vesicles was then extruded 29 times through a membrane with 200 nm diameter pores using an Avanti mini-extruder apparatus. This was done at room temperature, which was above the melting temperature of POPC (Tₘ=−2° C.).

Dynamic Light Scattering: To assess the quality of the liposome preparation, the POPC liposome stock solution was diluted to 1750 μM in D₂O, filtered with a 0.2 μm GHP filter into a glass test tube (200 mm×7 mm with 5 mm inner diameter) and multi-angle dynamic light scattering (DLS) was performed using a Brookhaven BI-200SM instrument with a 637 nm laser and a refractive index matching bath. Autocorrelation functions were recorded for 4 min, at angles of 60°, 75°, 90°, 105°, and 120° over a delay time range of 1 μs-100 ms. The data were then fit to the second-order cumulant expansion model and the bi-exponential model, and in all cases the second-order cumulant expansion model yielded equally random residuals with fewer fitting parameters, indicating a superior fit and therefore a single relaxation mode.

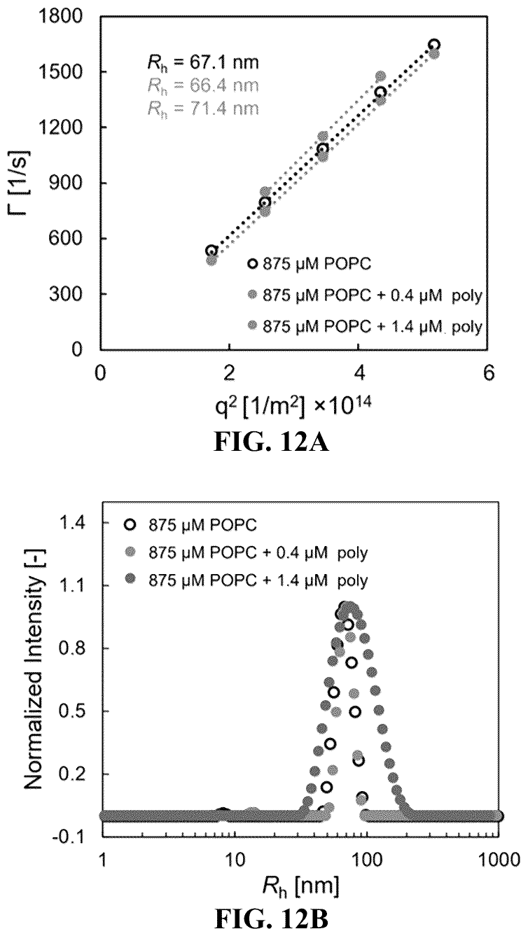

Pulsed-field-gradient-NMR (PFG-NMR) binding assay: A 1750 μM POPC liposome stock solution and a 28 μM polymer stock solution were prepared in D20. These stock solutions were then combined in a 1:1 volume ratio to yield the PFG-NMR sample with 875 μM POPC and 14 μM polymer. The polymer and lipid samples were incubated at room temperature for a minimum of 4 h to ensure that a steady state was reached. Polymer-only control samples were prepared by diluting the polymer stock with D₂O to a final concentration of 14 μM. FIG. 14 shows a non-limiting assessment of BBP stability in D₂O.

PFG-NMR experiments were conducted on a Bruker Avance III 500 MHz spectrometer equipped with a 5 mm TBO triple resonance PFG probe. The ledbpg2s sequence was performed with 32 scans, an acquisition time and delay time of Is, a diffusion time of 700 ms, a gradient pulse duration of 5 ms, and a gradient strength ranging from 2-95%. All measurements were taken at 27° C. The PEO methylene signal was analyzed in all cases because it has the highest signal-to-noise ratio. Complete dissolution of all BBP samples was verified by the homogeneous, clear appearance of all samples, the existence of only two populations in the polymer-only control samples (free chains and micelles), and the ability of the spectrometer to properly lock and shim on the sample which would be hindered by any insoluble particulates.

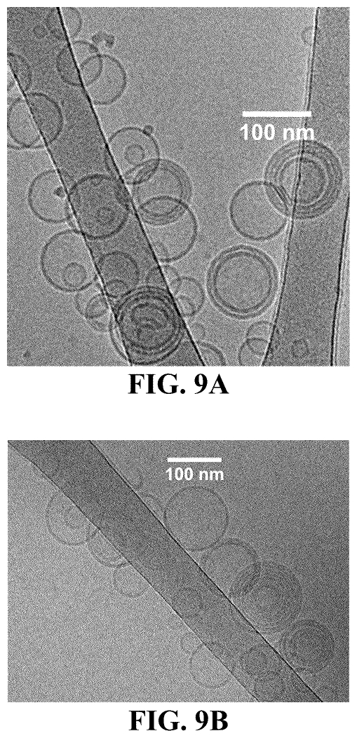

Cloud point measurements: Cloud point measurements were performed on a home-built apparatus. A 10 mW HeNe laser (633 nm) passes through a neutral density filter, the sample chamber, and then is focused by a lens onto a photodiode detector. Samples were sealed in glass ampoules and placed in the temperature-controlled sample chamber. Then, samples were heated from room temperature to 95° C. at a rate of 0.5° C./min, then a fan was turned on and the sample cooled by convection for another 2 h. The transmitted light was normalized by the maximum intensity during each sweep. The cloud point was determined to be the temperature at which the normalized transmittance falls below 75% on the heating trace. FIG. 10 shows non-limiting cloud point measurements.

Osmotic stress in vitro protection assay: C2C12 murine myoblasts were cultured using growth media (20% fetal bovine serum, 1% penicillin-streptomycin, 79% Dulbecco's modified Eagle medium) in a humidified chamber at 37° C. Cells were plated in a 96-well plate with 5000 cells per well and grown for 48 h. Isotonic (330 mOsm; 140 mM NaCl, 5 mM KCI, 2.5 mM CaCl₂, 2 mM MgCl₂, and 10 mM HEPES) and hypotonic (75 mOsm; 20 mM NaCl, 5 mM KCl, 2.5 mM CaCl₂, 2 mM MgCl₂, and 10 mM HEPES) buffers were prepared and adjusted to pH 7.2 by the addition of sodium hydroxide. Polymer solutions in isotonic and hypotonic buffer were prepared and diluted as needed. Once grown to approximately 70% confluency, growth media was removed from the cells; and 100 μL of isotonic buffer solutions, with or without polymer, was added to the cells for 30 min. Next, hypotonic buffer solutions replaced the isotonic buffer for 50 min. This was followed by an isotonic recovery period where hypotonic buffer was removed, and isotonic buffer solutions were added for 30 min. Lastly, the isotonic buffer was removed, and cells were lysed with 0.01% Triton X-100 for 50 min to ensure total lactate dehydrogenase (LDH) release.

Supernatants from each step (isotonic, hypotonic, isotonic recovery, and Triton) were retained, and the amount of LDH present was quantified using the LDH assay kit. The quantity of LDH in the supernatants is indicative of membrane integrity: if the membrane is damaged, LDH will leak out of the cell into the solution. The assay utilizes the fact that LDH catalyzes the reduction of nicotinamide adenine dinucleotide (NAD) to NADH, which absorbs light at 340 nm. Thus, the rate of increase in light absorption over 120 min is related to the concentration of LDH present in the supernatant. The total amount of LDH released is calculated per well, and the percentage of total LDH for each step is then normalized to the corresponding step in the untreated control (no polymer). This normalization can result in percent LDH releases above 100%, indicating more LDH is released in the treated sample than the untreated control. In this assay, the cell membrane is stretched during the hypoosmotic step as the cell swells; however, maximal LDH release occurs during isotonic recovery due to the net flow of solution out of the cell. Therefore, data from the isotonic recovery step is presented here because it best distinguishes performance differences among the polymers. In vitro experiments were carried out on three unique cell passages with three technical replicates each for a total of 9 independent replicates.

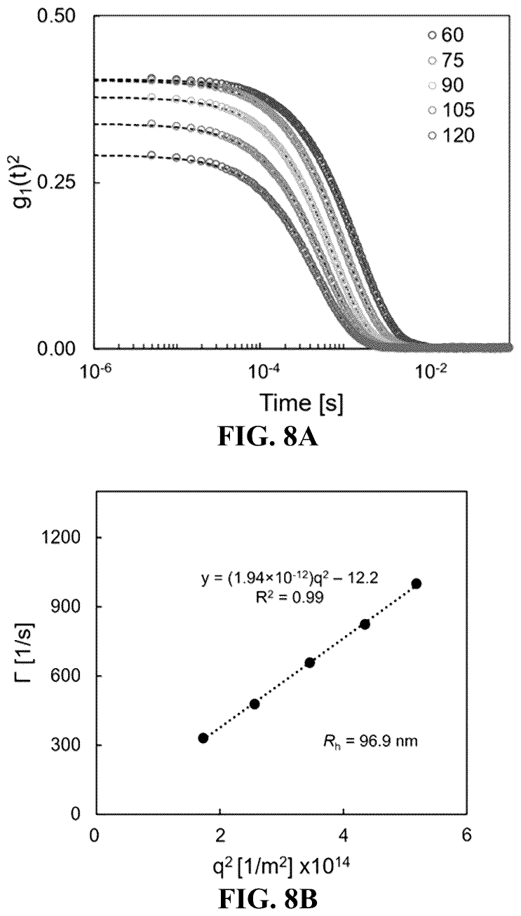

Cryo-Transmission Electron Microscopy: Cryo-TEM samples were prepared inside a Vitrobot system (Thermo Fisher Scientific) kept at room temperature and 100% humidity. A 3 μL drop of sample solution was placed onto a perforated carbon support film on a copper TEM grid (lacey Formvar/carbon films on 200 mesh Cu grids, Ted Pella, Inc., Redding, CA, U.S.A), held by tweezers inside the Vitrobot, followed by blotting the sample with a filter paper into a thin film, and plunging into liquid ethane for vitrification. Before sample preparation, the TEM grids were cleaned with glow-discharge air-plasma (PELCO EasiGlow™, Ted Pella, Inc.) to increase the support film hydrophilicity. Imaging was performed with a FEI Tecnai G2 F30 field emission gun TEM operated with an acceleration voltage of 300 kV, and using a Gatan 626 cryo-holder, kept at −175° C. Images were recorded digitally using a Gatan UltraScan 4000 4k×4k CCD camera. FIG. 9A-D shows representative cryo-TEM micrographs from neat POPC liposomes and POPC liposomes treated with a bottlebrush polymer.

Cryo-TEM micrographs (e.g., as in FIG. 9A-D) were used to assess the fraction of the lipid surface area that is in the outer leaflet, as well as to obtain a real-space image of a BBP bound to a liposome. By analyzing objects from 5 representative TEM micrographs of neat POPC liposomes, each observed bilayer was fit with the circle tool in ImageJ to calculate the surface area. From this procedure, about 68% of the liposome surface area was estimated to be on the outer leaflet and exposed to the polymer solution. This represents the parameter f in Eq. 3A below. In the TEM micrographs of liposomes treated with B-E₁₆₈⁴³P₄₃¹⁵, protrusions from the membrane were not observed. Furthermore, broken or open liposomes were not observed, which suggests that prolonged exposure to high Mₙ BBPs is not disruptive to the lipid bilayer structure.

### Example 3

### Polymer-Lipid Bilayer Association of Linear and Bottlebrush Poloxamers

Single-component POPC liposomes were used as model cell membranes to probe polymer-lipid bilayer binding. This model has been applied extensively because POPC is the most abundant lipid in the exterior membrane of mammalian cells, so this simplified model best balances the need for efficiency and physiological relevance. The nomenclature adopted for the BBPs is B-E_{N}_{_{bb}}^{N}^{_{sc}}P_{N}_{_{bb}}^{N}^{_{sc}}, where B indicates the brush architecture, E the PEO block, P the PPO block, and N_{sc} and N_{bb} are the average side chain and backbone degrees of polymerization, respectively.

Liposome size and dispersity were assessed via multi-angle DLS. The autocorrelation function from each angle was fit to the second-order cumulant expansion model shown in Eq. 1A:

$${g}_{1} ( t ) = exp ( - 2 Γ t + {k}_{2} {t}^{2} ) , \qquad ( 1 A )$$

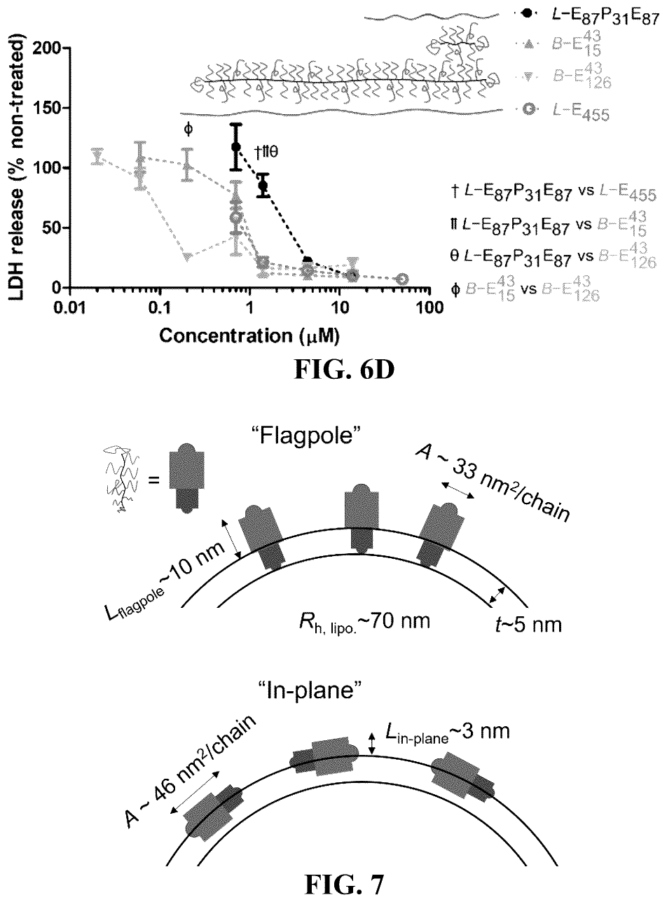

and a representative Rₕ and Ð for the liposomes is 97 nm and 1.03, respectively, consistent with the extrusion protocol. In Eq. 1A, tis time, I′and k₂ are the first and second cumulants which capture the mean and width of the relaxation rate in g₁, respectively. In brief, DLS analysis included converting the measured intensity autocorrelation function (g₂(1)) to the electric field autocorrelation function (g₁(1)) via the Siegert relation (see FIG. 8A). Then, the autocorrelation function was fit to the second-order cumulant expansion model described by Eq. 1A. The mutual diffusion coefficient (Dₘ) can be obtained by dividing I′ by the slope of the relationship between I′ and q² shown in FIG. 8B. For dilute solutions, the mutual diffusion coefficient is approximately equal to the tracer diffusion coefficient (Dₜ), which can then be related to the hydrodynamic radius (Rₕ) of the particles via the Stokes-Einstein equation given by Eq. 1B:

$${R}_{h} = \frac{kT}{6 {πη}_{s} {D}_{t}} , \qquad ( 1 B )$$

where k is the Boltzmann's constant, Tis the temperature in Kelvin, and ηₛ is the viscosity of the solvent. A representative value for Rₕ of the liposomes is 97 nm, which is consistent with the pore sized used during extrusion. Finally, the dispersity (Ð) of the liposomes can be estimated by Eq. 1C:

$$Ð = 1 + {k}_{2} / {Γ}^{2} . \qquad ( 1 C )$$

The linearity and y-intercept of zero (within error) of the relationship between the first cumulant (I′) and the scattering vector squared (q²) indicate that a single diffusive process was observed.

After verifying liposome quality, the liposomes were incubated with the polymer of interest for at least 4 h to allow the system to approach a steady state. PFG-NMR spectroscopy was then applied to quantify the fraction of chains bound to the liposomes. In a PFG-NMR experiment, the intensity of all proton echo signals decays exponentially as the magnetic field gradient strength (G) increases. The rate of signal attenuation is related to the translational diffusion coefficient through Eq. 2A:

$$\frac{I}{{I}_{0}} = exp ( - {γ}^{2} {δ}^{2} {G}^{2} D ( Δ - \frac{δ}{3} ) ) , \qquad ( 2 A )$$

where I is the signal intensity at the given gradient strength, I₀ is the signal intensity with no gradient, γ is the gyromagnetic ration of a proton (42.6 MHz/T), G is the magnetic field gradient strength (2-95%), D is the translational diffusion coefficient of the species contributing to the selected ¹H NMR signal, Δ is the diffusion time (700 ms), and δ is the pulse duration (5 ms). Once D is determined, the Stokes-Einstein equation (Eq. 1B) can be applied to calculate Rₕ. In resolving free polymer and liposome-bound polymer, when multiple populations are present, if the length scales of the different states differ by more than a factor of ˜2, they can be resolved as separate rates of decay. In this case, the PFG-NMR data can be described by an expansion of exponentials shown by Eq. 2B:

$$\frac{I}{{I}_{0}} = {\sum }_{i = 1}^{n} {f}_{i} exp ( - {γ}^{2} {δ}^{2} {G}^{2} {D}_{i} ( Δ - \frac{δ}{3} ) ) , \qquad ( 2 B )$$

where fᵢ and Dᵢ are the mole fraction and diffusion coefficient of the i^{th} species.

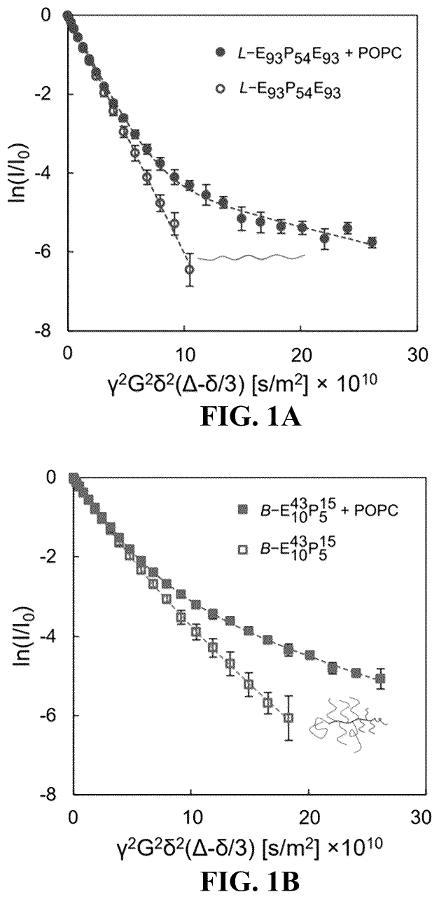

FIG. 1A shows the PFG-NMR results for the commercial poloxamer F127 (L-E₉₃P₅₄E₉₃), which is a linear poloxamer. When the natural logarithm of the normalized intensity is plotted against gradient strength, the magnitude of the slope of the decay curve gives the diffusion coefficient (D). In FIG. 1A, the decay curve for the polymer-only control (open symbols) is linear and can be described by a single exponential decay, indicating a single population with Rₕ=3.1±0.1 nm, which is consistent with the estimate for L-E₉₃P₅₄E₉₃ if it were a Gaussian coil (˜2.8 nm). Polymer binding to the liposomes can be seen by comparing the polymer-only control to the polymer+POPC decay curves. In the latter, polymer binding to liposomes results in the onset of a second regime with a weaker slope at high values of the x-coordinate. Fitting the decay curve to a two-term expansion of Eq. 2B (bi-exponential model) yielded an estimate of 2.3±0.9 mol % of the L-E₉₃P₅₄E₉₃ chains bound to the liposomes with an Rₕ=26±6.1 nm.

There is a discrepancy between the size estimates for the liposomes from PFG-NMR and DLS. This has been observed in the past and is attributed to a combination of two factors: (i) DLS emphasizing large species because they scatter light more intensely, and (ii) PFG-NMR underestimating the size of liposomes because NMR signals from the larger liposomes in the distribution relax more slowly, leading to signal broadening. Analysis of cryo-TEM images of liposomes extruded through a 200 nm diameter membrane yielded an estimate for the liposome radius of 63±29 nm, which is intermediate to the estimates from PFG-NMR and DLS.

FIG. 1B shows the PFG-NMR decay curves for B-E₁₀⁴³P₅¹⁵, a BBP with a similar weight fraction of PEO and a similar number of PPO units as I-E₉₃P₅₄E₉₃. Note that a direct comparison between the linear and bottlebrush architectures where only one variable is changed is impossible, due to the backbone chemistry being unique to the bottlebrush architecture. Therefore, the number of PPO units was chosen as the most appropriate variable to hold constant because the anchor and chain model of polymer-lipid interactions asserts that the PPO moiety is the primary driver of binding. The first impact of the architectural change can be seen by comparing the two polymer-only control curves. The decay curve corresponding to B-E₁₀⁴³P₅¹⁵ is non-linear, indicating the coexistence of two populations with distinct sizes. Fitting the decay curve to the bi-exponential model indicated that the sizes of the populations differ by a factor of two, which is attributed to a coexistence of free chains (R_{h, free chains}=3.9±0.5 nm) and micelles (R_{h, micelles}=8.0±1.5 nm). Because bottlebrush molecules adopt extended conformations, micelles are expected to be roughly twice as large as free chains (R_{h, micelle}˜2 R_{h, free chain}).

When liposomes are treated with B-E₁₀⁴³p₅¹⁵, the decay curve shows an additional, slower decay than the polymer-only control, indicating binding to liposomes. In this case, the polymer+POPC sample contained three distinct populations: free chains, micelles, and liposome-bound polymer. To reduce the number of fitting parameters without compromising accuracy, the decay curve was fit to a constrained three-term expansion of Eq. 2B (tri-exponential model). Since R_{h, free chains} and R_{h, micelles} are independent of liposome presence, the bi-exponential model fitting results from the polymer-only control were used to constrain the diffusion coefficients of these populations. The resulting fraction of polymer bound to liposomes is 7.3±1.3 mol % and R_{h, liposome}=25±11 nm. A Welch's T-test confirmed that the difference in bound fraction between the linear (2.3±0.9 mol %) and bottlebrush (7.3±1.3 mol %) poloxamers was statistically significant (p=0.012), while there is no difference in the Rₕ of the liposomes (p=0.20). The model fitting procedure described here was applied to all BBP+POPC samples. Due to the presence of micelles in the BBP samples, relatively large liposomes were used to increase the difference in size between the micelle and bound states, particularly as the backbone degree of polymerization of the BBP is increased. Without wishing to be limited by mechanism, decreasing the liposome radius has been shown to increase the fraction of linear poloxamer that binds to the bilayer due to the increase in membrane curvature. A similar trend could exist in BBPs; however, this is experimentally challenging due to the presence of micelles. To compare the binding ability of bottlebrush to linear poloxamers and to explore the bottlebrush parameter space, a single liposome size (extrusion diameter=200 nm) was chosen.

FIG. 1C summarizes the number fraction of chains in each state for all four decay curves. The polymer-only control for L-E₉₃P₅₄E₉₃ shows a single exponential decay, confirming no micellization at 14 μM. Adding liposomes leads to 2.3 mol % of the linear poloxamer chains binding to the bilayer vesicles. In comparison, the polymer-only control for the analogous BBP does micellize at 14 μM with 27 mol % of chains forming micelles. The fitting results for the B-E₁₀⁴³P₅¹⁵+POPC sample suggest 21 mol % of chains exist in micelles and 7.3 mol % are bound to the liposomes. A complete summary of the fitting parameters are shown in Table 3A below.

TABLE 3A
Summary of the summation of exponentials model for all polymer + liposome samples

| Polymer | f_{micelle} [mol %] | f_{bound} [mol %] | R_{h, free chain} [nm] | R_{h, micelle} [nm] | R_{h, liposome} [nm] | CMC^{a} [mg/mL] | CMC^{a} [μM] |
|---|---|---|---|---|---|---|---|
| L-E₉₃P₅₄E₉₃ (F127) | 0 | N/A | 3.1 ± 0.1 | — | N/A | 0.5 ± 0.1 | 40 ± 10 |
| L-E₉₃P₅₄E₉₃ (F127) + POPC | 0 | 2.3 ± 0.9* | 3.2 ± 0.1* | — | 26.0 ± 6.1* | | |
| B-E₁₀⁴³P₅¹⁵ | 26.8 ± 9.3 | N/A | 3.9 ± 0.5 | 8.0 ± 1.5 | N/A | 8 ± 3 | 300 ± 100 |
| B-E₁₀⁴³P₅¹⁵ + POPC | 20.5 ± 8.6 | 6.8 ± 1.5 | 3.9 ± 0.5 | 8.0 ± 1.5 | 25.4 ± 10.8 | | |
| B-E₂₁⁴³P₁₁¹⁵ | 38.9 ± 2.1 | N/A | 6.3 ± 0.6 | 12.7 ± 0.7 | N/A | 2 ± 0.5 | 40 ± 10 |
| B-E₂₁⁴³P₁₁¹⁵ + POPC | 56.4 ± 22.9* | 0 | 6.4 ± 0.5* | 13.2 ± 1.3* | — | | |
| B-E₅₄¹⁰P₈¹⁵ | 83.3 | N/A | 2.5 | 12.0 | N/A | 0.4 ± 0.1 | 30 ± 10 |
| B-E₅₄¹⁰P₈¹⁵ + POPC | 83.5* | 0 | 1.7* | 11.0* | — | | |
| B-E₄₀¹⁰P₇¹⁵ | 80.0 ± 9.3 | N/A | 2.6 ± 0.3 | 12.5 ± 1.4 | N/A | | |
| B-E₄₀¹⁰P₇¹⁵ + POPC | 77.2 ± 11.1* | 0 | 2.4 ± 0.9* | 12.5 ± 1.8* | — | | |
| B-E₄₀¹⁰-s-P₇¹⁵ | 0 | N/A | 4.2 ± 0.1 | — | N/A | | |
| B-E₄₀¹⁰-s-P₇¹⁵ + POPC | 0 | 0 | 4.5 ± 0.1* | — | — | | |
| B-E₁₁⁴³P₆¹⁵ | 17.8 ± 11.7 | N/A | 4.4 ± 0.1 | 11.2 ± 0.7 | N/A | | |
| B-E₁₁⁴³P₆¹⁵ + POPC | 28.5 ± 12.5 | 8.3 ± 4.1 | 4.4 ± 0.1 | 11.2 ± 0.7 | 28.4 ± 11.7 | | |
| B-E₁₂⁴³-s-P₆¹⁵ | 0 | N/A | 4.7 | — | N/A | | |
| B-E₁₂⁴³-s-P₆¹⁵ + POPC | 5.1 ± 5.1* | 0 | 4.6 ± 0.1* | 10.0* | — | | |
| L-E₄₅₅ | 0 | N/A | 4.3 | — | N/A | — | — |
| L-E₄₅₅ + POPC | 0 | 0 | 4.6* | — | — | | |
| B-E₁₅⁴³ | 0 | N/A | 3.9 | — | N/A | | |
| B-E₁₅⁴³ + POPC | 0 | 0 | 3.7 ± 0.1 | — | — | | |

For Table 3A, all polymer+liposome fits were done with the constrained tri-exponential model as described in the main text unless otherwise noted. All polymer-only fits were done with the bi-exponential model. In several cases, this model collapses to the single exponential model. * indicates an unconstrained bi-exponential model; error is the standard deviation of two independent replicates for the statistical vs. linear molecules and three independent replicates elsewhere; and ^{a}indicates that CMC was measured at 37° C. via dynamic light scattering.

Firstly, these results show that, after the introduction of liposomes, the fraction of BBP chains in the micellar state decreases while the fraction of free chains remains the same. Without wishing to be limited by mechanism, this suggests that polymer binding to the liposome perturbs the equilibrium between free chains and micelles, at which point chain expulsion from existing micelles re-establishes equilibrium. The significant population of free chains indicates that the barrier to chain exchange is small. Furthermore, chain exchange between micelles formed by linear-b-brush polymers with the brush block in the core has been shown to occur relatively rapidly. Secondly, the three-fold increase in the bound fraction of B-E₁₀⁴³p₅¹⁵ compared to L-E₉₃P₅₄E₉₃ was surprising given the relative rigidity and steric bulk of the brush architecture. To better understand what causes this increase in membrane affinity, various characteristics were systematically varied, including polymer composition, overall molecular weight, side chain length asymmetry, and compared statistical and block copolymers. A summary of characteristics of the synthesized polymers is given in Table 3B. Every polymerization went to complete conversion, yielded low dispersity polymer (Ð<1.10), contained no residual catalyst (absence of an NMR signal at ˜20-21 ppm), and was very close to the targeted composition.

TABLE 3B
Polymer characteristics

| | Mₙ | | wt % | wt % | | |
|---|---|---|---|---|---|---|
| Polymer | [kDa]^{a} | Ð^{a} | PEO^{b} | PPO^{b} | N_{PEO}^{c} | N_{PPO}^{c} |
| L-E₈₇P₃₁E₈₇ (P188) | 9.4 | 1.19 | 81 | 19 | 170 | 31 |
| L-E₉₃P₅₄E₉₃ (F127) | 11.3 | 1.14 | 73 | 27 | 190 | 54 |
| B-E₁₀⁴³P₅¹⁵ | 26.2 | 1.07 | 73 | 19 | 430 | 78 |
| B-E₂₁⁴³P₁₁¹⁵ | 55.3 | 1.10 | 72* | 20* | 910 | 160 |
| B-E₁₆₈⁴³P₄₃¹⁵ | 390 | 1.15 | 78 | 15 | 6900 | 910 |
| B-E₅₄¹⁰P₈¹⁵ | 40.9 | 1.09 | 62 | 18 | 530 | 120 |
| B-E₁₁⁴³P₆¹⁵ | 29.4 | 1.06 | 72 | 20 | 470 | 94 |
| B-E₁₂⁴³-s-P₆¹⁵ | 32.0 | 1.06 | 75 | 17 | 530 | 90 |
| B-E₄₀¹⁰P₇¹⁵ | 31.5 | 1.09 | 59 | 21 | 390 | 100 |
| B-E₄₀¹⁰-s-P₇¹⁵ | 30.9 | 1.04 | 60 | 20 | 390 | 100 |
| L-E₄₅₅ | 20.0 | 1.10 | 100 | 0 | 460 | 0 |
| B-E₁₅⁴³ | 30.6 | 1.06 | 93 | 0 | 640 | 0 |
| B-E₁₂₆⁴³ | 260 | 1.08 | 93 | 0 | 5400 | 0 |
| B-E₅₇⁴³P₆¹⁵ | 120 | 1.04 | 93 | 6 | 2400 | 94 |
| B-E₁₁₇⁴³P₆¹⁵ | 250 | 1.07 | 93 | 3 | 5000 | 94 |

In Table 3B, ^{a}indicates determined from SEC(MALS+dRI); ^{b}indicates estimated from ¹H NMR spectra and knowledge of the macromonomer molecular weights after functionalization with the norbornene (MALDI-ToF) unless otherwise noted; *indicates composition reported from SEC; and ^{c}indicates Mₙ for both blocks was calculated as the product of the weight fraction of that block (including the backbone mass) and the overall molecular weight. The number of grafts in both blocks was then estimated by dividing the Mₙ of each block by the corresponding macromonomer molecular weight. Finally, the number of PEO/PPO units in the chain was calculated by the product of the number of grafts and the average number of PEO/PPO repeat units in each graft as determined via MALDI-ToF.

### Example 4

### Effect of the Hydrophobic Backbone

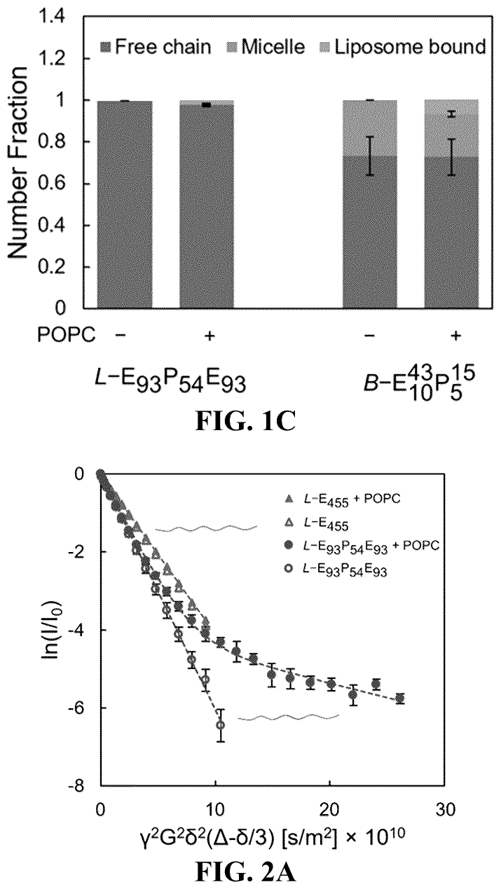

Linear PEO homopolymers with Mₙ<100 kDa did not bind to liposomes to a measurable extent, indicating that the PPO block may be useful for membrane association (FIG. 2A). Without wishing to be limited by mechanism, this may be be because PEO is too hydrophilic to drive a binding event on its own at 27° C. As shown in Scheme 4A, BBPs contain a norbornene backbone, which constitutes another source of hydrophobicity that could drive binding.

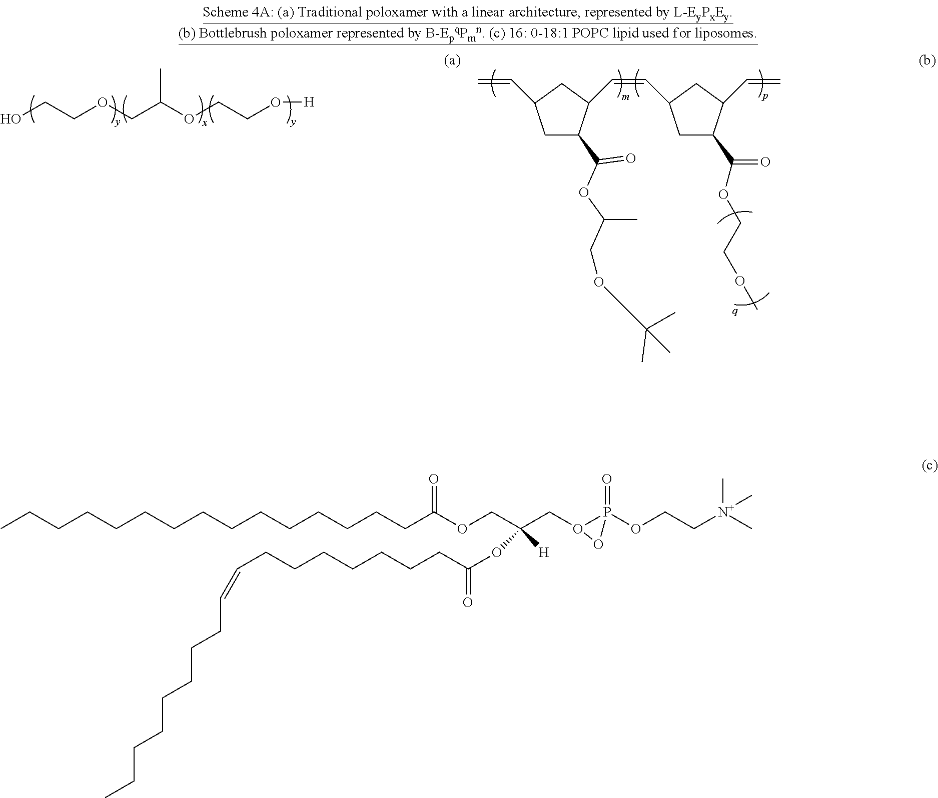

To evaluate this possibility, B-E₁₅⁴³ was synthesized, and this polymer has the same total backbone degree of polymerization as the diblock discussed above (B-E₁₀⁴³P₅¹⁵) but with only PEO side chains. FIG. 2B compares the PFG-NMR data for this PEO bottlebrush homopolymer with the corresponding PEO-PPO bottlebrush diblock polymer (B-E₁₀⁴³P₅¹⁵). The superposition of the polymer-only control and the polymer+POPC decay curves in the brush PEO homopolymer case indicates that B-E₁₅⁴³ does not bind to the liposomes at a detectable level. Therefore, in some non-limiting cases, the hydrophobicity of the backbone alone may not be sufficient to drive binding. Furthermore, a relatively hydrophobic PPO brush block may be useful to promote binding in some non-limiting instances. Without wishing to be limited by theory, this likely reflects the steric shielding of the backbone by the PEO side chains, which could prevent the backbone from entering the acyl region of the bilayer and/or limit solvent-backbone contact.

To assess the ability of water to access the backbone, the cloud point of L-E₄₅₅ and B-E₁₅⁴³ was measured to compare the hydrophilicity of these molecules. In an aqueous buffer at 3 mg/mL, neither of these solutions turn cloudy below 100° C. Then, cloud point measurements were performed in a 1 M potassium fluoride solution, as this salt is known to depress the cloud point of linear PEO to a more convenient temperature range. The bottlebrush PEO polymer had a cloud point of 48° C. and the linear PEO had a cloud point of 60° C. (FIG. 10). This indicates that the backbone may slightly increase the hydrophobicity of the bottlebrush polymer. Without wishing to be limited by theory, water may be able to access the backbone to some extent. Thus, the steric hindrance of the sidechains could prevent the added hydrophobicity of the backbone from affecting membrane binding, and there may be hydrophobicity on the periphery of the molecule for membrane binding to occur. Because the backbone is less likely to insert on its own, it cannot explain the increase in membrane affinity of the BBP compared to the linear poloxamer.

### Example 5

### Molecular Weight Effect

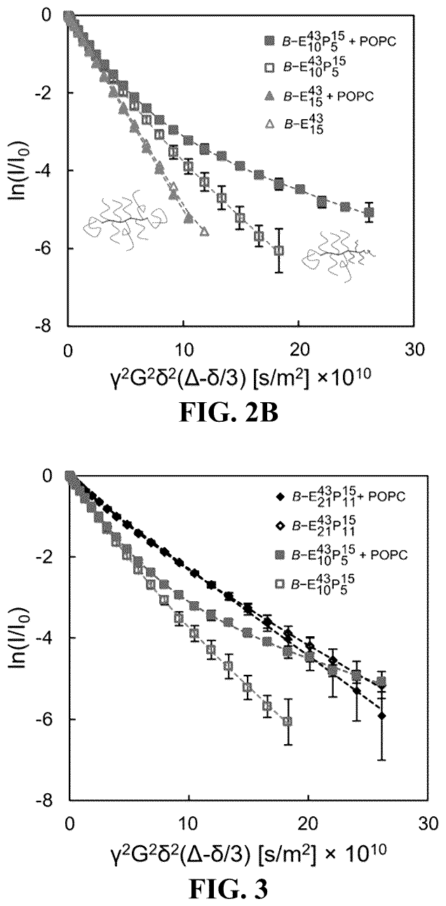

The BBP discussed above (B-E₁₀⁴³P₅¹⁵) had roughly double the molecular weight of L-E₉₃P₅₄E₉₃, the linear poloxamer control in FIG. 1A-C. Due to the molecular weights of the macromonomers (˜1 kDa for PPO and ˜2 kDa for PEO), synthesizing a diblock BBP at a molecular weight equal to L-E₉₃P₅₄E₉₃ would require degrees of polymerization of only 5 and 2 for the PEO and PPO blocks, respectively. These two molecules do have similar numbers of total PPO units, 54 for L-E₉₃P₅₄E₉₃ and 78 for B-E₁₀⁴³P₅¹⁵. Since the PPO moieties could affect binding events, comparing these molecules may be useful. However, for linear poloxamers, increasing the molecular weight can increase the extent of binding, so the effect of overall molecular weight could be explored in the bottlebrush architecture as well. To this end, B-E₁₀⁴³P₅¹⁵ and B-E-₂₁⁴³P₁₁¹⁵ were synthesized. These two BBPs have identical compositions and side chain lengths but have molecular weights that differ by a factor of ˜2. The PFG-NMR binding assay results for these two polymers are shown in FIG. 3. For the higher molecular weight BBP (black), the decay curves of the polymer-only control and the polymer +POPC sample are equivalent within uncertainty. Therefore, both decay curves were fit to the bi-exponential model and the Rₕ of the small and large species differ by a factor of two. Thus, the large species detected in the B-E₂₁⁴³P₁₁¹⁵+POPC sample is micelles. Finally, since the size of the B-E₂₁⁴³P₁₁¹⁵ micelles (13±1.3 nm) is significantly smaller than the Rₕ of the liposome-bound species detected in the L-E₉₃P₅₄E₉₃+POPC (R_{h, liposome}=26±6.1 nm) and B-E₁₀⁴³P₅¹⁵+POPC (R_{h, liposome}=25±11 nm) samples, if this polymer did bind to the liposomes the PFG-NMR assay would have detected an additional mode of relaxation. The B-E₂₁⁴³P₁₁¹⁵ chains likely do not bind to liposomes to a detectable extent. When the molecular weight of a BBP is increased, the size of the micelles increases. At an Mₙ˜100 kDa, the length scales of the micelles and liposomes are commensurate, and therefore the PFG-NMR binding assay cannot discriminate between them. Given the effect of Mₙ between 26 kDa and 55 kDa, BBPs with Mₙ>55 kDa could also have very low membrane affinity.

### Example 6

### Role of PEO Block Side Chain Length

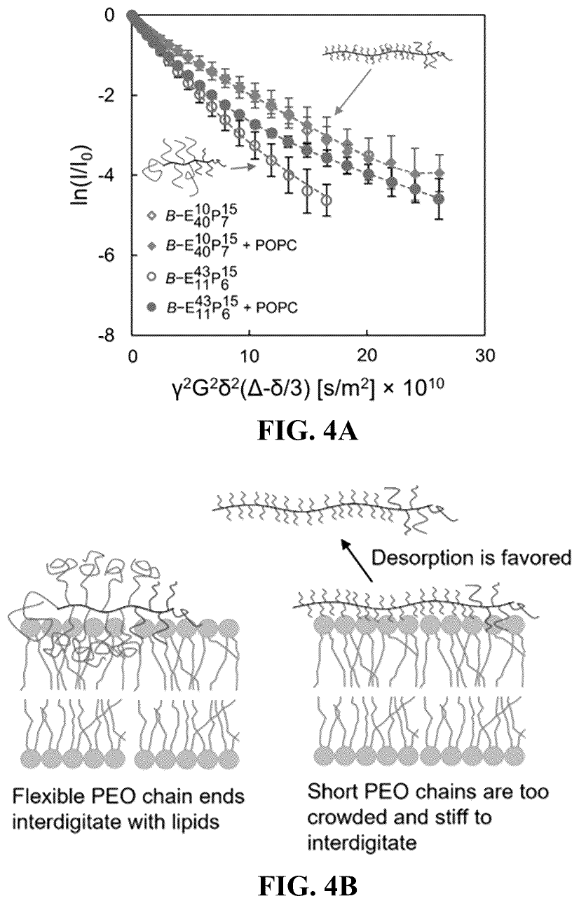

The side chain length of bottlebrush polymers could impact properties such as shape and side chain flexibility, which may influence polymer-membrane interactions. Thus, the effect of the PEO block side chain length was evaluated by comparing the binding ability of B-E₁₁⁴³P₆¹⁵ and B-E₄₀¹⁰P₇¹⁵. For these two BBPs, the hydrophobic block is nearly identical, but the hydrophilic block side chain and backbone lengths differ by a factor of four. Notably, when N_{sc, PEO} was reduced, N_{bb, PEO} was increased to maintain a roughly constant number of EO units. The PFG-NMR decay curves for B-E₁₁⁴³P₆¹⁵ (open and closed circles in FIG. 4A) show a shift to the upper right when liposomes are present, indicating binding. The tri-exponential fit reveals that roughly 8±4 mol % of the chains bound while 29±13 mol % existed as micelles; these results are consistent with those of B-E₁₀⁴³P₅¹⁵ from FIG. 1A-C, which is a very similar molecule. The PFG-NMR data for B-E₄₀¹⁰P₇¹⁵ (open and closed diamonds in FIG. 4A) reveal that shortening the PEO block side chains and lengthening the PEO backbone changes the extent of micellization and the membrane affinity of the polymer. First, fitting the polymer-only control decay curve to the bi-exponential model revealed that 80±9 mol % of chains exist as micelles. This is much higher than the micelle fraction of the B-E₁₁⁴³P₆¹⁵ polymer-only control, which is only 18±12 mol %. Second, the polymer-only control and the polymer+POPC sample for B-E₄₀¹⁰P₇¹⁵ (open and closed diamonds) overlap, indicating that no binding was detected despite the increased hydrophobicity of this molecule (lower wt % PEO). This result demonstrates that the PEO block may play a role in binding to liposomes. As the PEO side chain length is decreased, the units most distal from the backbone could have less space and may therefore be less flexible. Bottlebrush molecules with reduced flexibility at the side chain ends may form smaller, looser aggregates due to reduced side chain interdigitation. Without wishing to be limited by theory, a similar effect may occur in BBP interactions with lipid membranes. In some non-limiting cases, the side chains of the PPO block may be the first moiety of the BBP to insert into the bilayer. This could bring the PEO block close to the lipid bilayer surface. If the PEO side chains are sufficiently long and flexible, they could interdigitate between lipid molecules, which can disrupt the water shell around the ether units, enabling the PEO block to provide an additional anchoring effect. On the other hand, short PEO side chains may be too sterically constrained by their neighbors to interact with the lipid headgroups, and therefore steric repulsion between the PEO brush block and the lipids could lead to rapid desorption. FIG. 4B shows a non-limiting schematic of this hypothesized interpretation of the effect of the PEO side chain length.

### Example 7

### Comparison of Statistical Versus Block BBPs

In certain studies, a minimum PPO block length for membrane insertion of a linear poloxamer was determined to be ˜14 units. The number-average degree of polymerization of the PPO side chains in the BBPs tested met this threshold. Furthermore, each PPO side chain could serve as a pseudo-independent anchoring site, thereby increasing the membrane affinity. To test this hypothesis, two pairs of molecules with different PEO side chain lengths were synthesized, where each pair contains one statistical copolymer and one block copolymer of nearly identical composition and molecular weight.

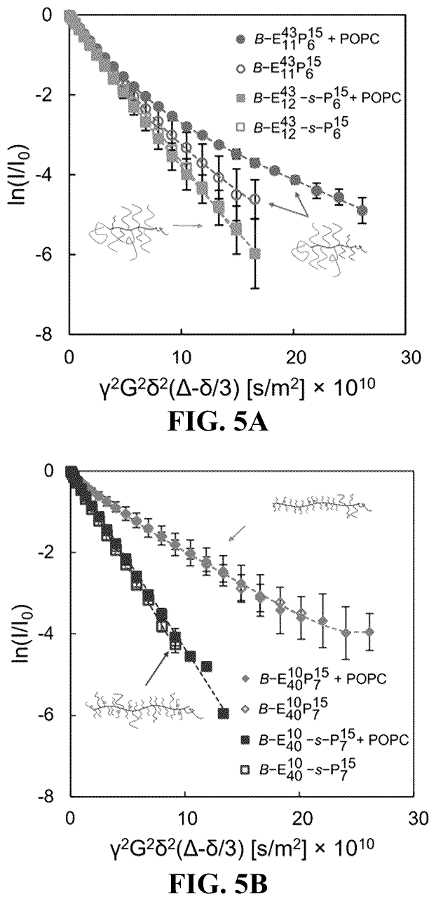

The PFG-NMR results for the statistical/block pair with long and short PEO side chains are shown in FIG. 5A and FIG. 5B, respectively. The polymer-only control for the block copolymer with long PEO side chains (open circles in FIG. 5A) showed a curvature indicating micellization, and the bi-exponential fitting revealed that 18±12 mol % of chains are in micelles. In contrast, the decay curve of the polymer-only control corresponding to the statistical copolymer with long PEO side chains (open and closed squares in FIG. 5A) was essentially linear. In this case, the bi-exponential model estimated only 5±5 mol % in micelles. The reduction of the micelle population is indirect evidence that the copolymerization of the PPO and PEO macromonomers was approximately random since the molecules are less able to organize into a core-shell structure.

Comparing the decay curves of corresponding polymer+POPC and polymer-only samples in FIG. 5A demonstrates that the block architecture binds to liposomes. The tri-exponential model fitting for the decay curve of B-E₁₁⁴³P₆¹⁵+POPC indicated that roughly 8±4 mol % of the chains bind to liposomes. This result suggested that when the PEO side chains are roughly three times longer than the PPO side chains, a block architecture may be useful for binding to occur. At this ratio of side chain lengths, the PPO chains may be screened by neighboring PEO chains in the statistical architecture, but not in the block architecture. Therefore, a statistical/block pair of copolymers with PPO side chains that are roughly twice the length of the PEO side chains were synthesized to test whether removing the steric constraint allowed a statistical copolymer to bind.

FIG. 5B shows the decay curves of block and statistical copolymers with long PPO side chains relative to the PEO side chains. In this case, the statistical and block copolymers both showed overlap between the polymer-only and polymer+POPC decay curves, indicating that neither architecture produced binding to liposomes. Therefore, reducing the steric hindrance of the PPO units on the periphery of the molecule, in this non-limiting case, may not result in binding of the statistical copolymer. Furthermore, in this non-limiting case, individual PPO side chains when statistically distributed along the entire backbone may not act as independent anchor sites. In some non-limiting cases, both a blocky architecture and a sufficiently long PEO side chain may be present in the polymer to promote binding to liposomes.

### Example 8

### In Vitro Protection Efficacy Against Osmotic Stress

Cellular protection efficacy of BBPs were evaluated and compared to linear analogues using an osmotic stress assay. Briefly, this assay subjects C2C12 myoblast cells to a hypoosmotic stress followed by isotonic recovery. If the membrane is damaged, macromolecules, such as the protein LDH, leak out of the cell and can then be detected via enzymatic colorimetry. A given polymer treatment is present throughout the assay, and a reduced LDH release relative to the non-polymer treated control indicates a protective effect.

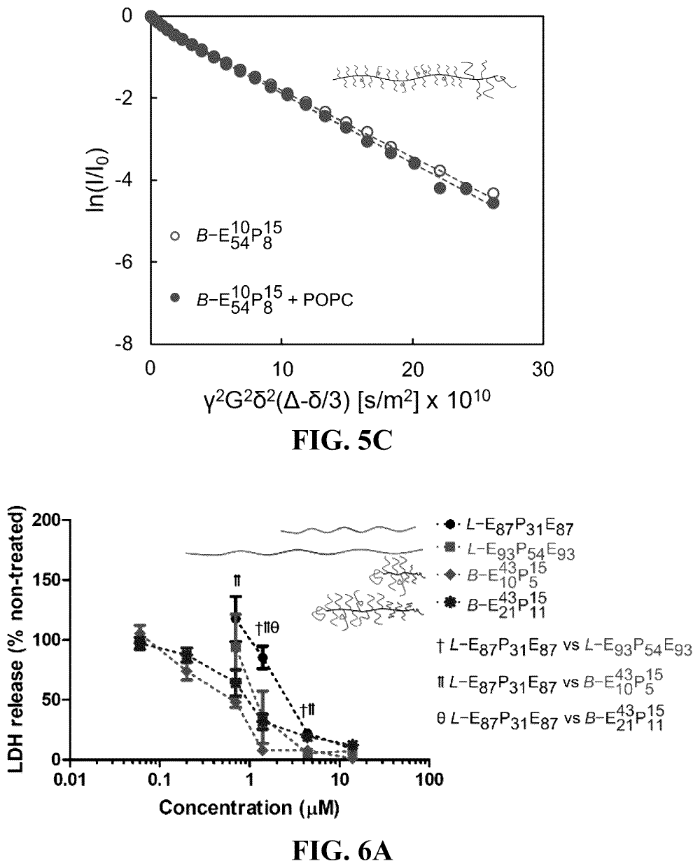

FIG. 6A shows a comparison of the linear and bottlebrush architectures, where L-E₈₇P₃₁E₈₇ (P188) was included as a “first-in-class” control. B-E₁₀⁴³P₅¹⁵ (diamond symbols) exhibits efficacy at sub-μM concentrations, indicating that BBPs can have a protective effect. Moreover, B-E₁₀⁴³P₅¹⁵ exhibited significantly greater cellular protection than the L-E₈₇P₃₁E₈₇ control (p<0.01) on a molar basis. To control for the difference in PEO mass fraction between B-E₁₀⁴³P₅¹⁵ and L-E₈₇P₃₁E₈₇, L-E₉₃P₅₄E₉₃ was included. L-E₉₃P₅₄E₉₃ is a linear poloxamer with an identical mass fraction of PEO and a similar number of PPO units to B-E₁₀⁴³P₅¹⁵; the protection efficacy of these two molecules is not statistically different at polymer concentrations >0.7 μM.

A comparison of B-E₁₀⁴³O₅¹⁵ and B-E₂₁⁴³P₁₁¹⁵ (curves for diamond and star symbols in FIG. 6A) indicated that doubling the molecular weight of the BBP nominally decreased protection efficacy. This effect was previously observed for high concentrations of linear poloxamers containing 70 and 80 wt % PEO and can be attributed to differences in the onset of micellization. Micellization likely cannot explain the reduced protection efficacy upon increasing Mₙ of BBPs, because all BBPs tested here form micelles over part of the concentration range tested, and no coincident reduction in protection efficacy was observed. Furthermore, as BBPs adsorb to the cell membrane, micelles can act as a reservoir by ejecting single chains to restore the free chain-micelle equilibrium.

A scaling theory by Hristova and Needham concerning mixtures of lipids and polymer-grafted lipids suggests that membrane mechanical properties are a function of both polymer molecular weight and concentration in the bilayer. Furthermore, their theory suggests that as the molecular weight of the chain is increased, the maximum possible membrane incorporation decreases. Therefore, without wishing to be limited by mechanism, a possible explanation for peak protection efficacy occurring at intermediate molecular weights (Mₙ˜10-30 kDa) is that as Mₙ is increased there are competing effects: less polymer binds, but each binding event adds more polymer mass to the membrane, leading to a larger effect on mechanical properties per binding event.

The effect of BBP architectural parameters on protection efficacy was explored. FIG. 6B shows that shortening the PEO side chains leads to a nominal improvement in protection efficacy. In particular, the BBP with shorter PEO side chains (B-E₅₄¹⁰P₈¹⁵) did not bind to a detectable level in the PFG-NMR assay (FIG. 5C), yet it does have a protective effect (which is also true for B-E₂₁⁴³P₁₁¹⁵ and the statistical BBPs). Without wishing to be limited by theory, this could be because the single component POPC liposome model neglects the effects of transmembrane proteins, surface receptors, glycolipids, and lipid phase coexistence that exist on a physiological membrane which could impact polymer-cell membrane interactions. Additionally, as temperature increases, one might expect binding to increase due to the reduced solvent quality of water for both PPO and PEO, so it is possible that by increasing the temperature from 27° C. to 37° C. in the PFG-NMR assay, binding of B-E₅₄¹⁰P₈¹⁵ and/or B-E₂₁⁴³P₁₁¹⁵ to liposomes would be observed. Furthermore, the relationship between abiotic membrane binding and cellular protection is poorly understood, so one should interpret the results of the liposomal PFG-NMR and the in vitro osmotic stress assays separately. Overall, these results show that low-affinity polymers can have a protective effect.

FIG. 6C indicates that protection efficacy may not be unique to the block architecture, as statistical copolymer bottlebrushes are also significantly more protective than L-E₈₇P₃₁E₈₇ on a molar basis. Furthermore, for the pair of molecules with long PEO side chains, the block and statistical architectures showed similar protection efficacy (closed squares vs. closed triangle symbols). However, when the PEO side chain length was shortened, the statistical copolymer performed significantly better than the corresponding block polymer at very low concentrations (red vs. blue points, p<0.01). Interestingly, it remained protective even down to 0.014 μM, which is the lowest protective polymer concentration of any polymer tested to date. All other molecules follow a threshold effect, where polymers lose protective efficacy somewhere between 0.8-4 μM. Future characterization could be conducted with any polymers herein, in which experiments can include a tissue-level protection assay with dystrophic mouse muscle fibers, which is a step closer to a physiological situation.

Finally, FIG. 6D shows that bottlebrush PEO polymers can be protective even without the PPO block. Additionally, a linear PEO homopolymer and a brush PEO polymer of similar molecular weights have the same protection efficacy. At low polymer concentrations, increasing the molecular weight of the brush PEO homopolymer from 30 kDa to over 200 kDa significantly improves the per molecule protection efficacy (p<0.01). As discussed above, it may be unlikely that the backbone alone imparts sufficient hydrophobicity to affect binding. Therefore, these results suggest that polymers that only weakly associate with POPC liposomes can be protective of cellular membranes.

### Example 9

### Discussion

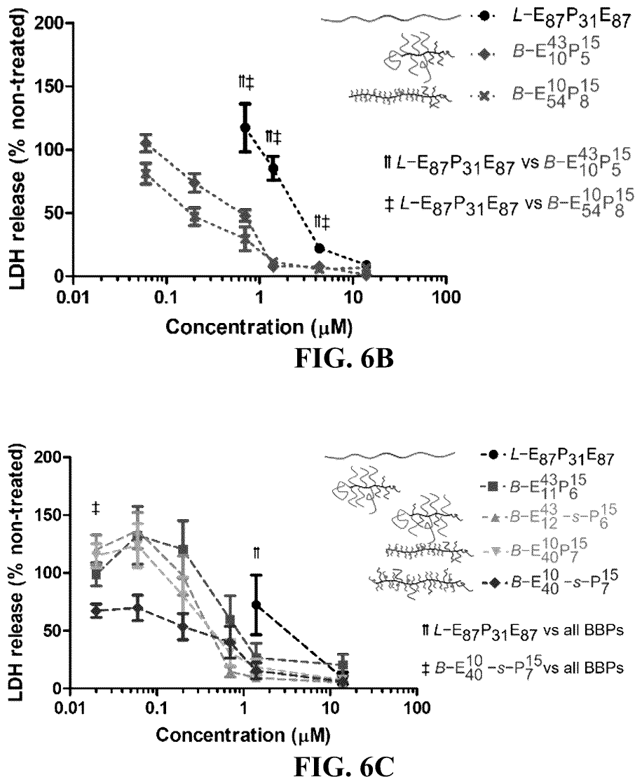

The fraction of chains in micelles and bound to liposomes for all polymer+POPC PFG-NMR experiments are summarized in Table 9A. Using the PFG-NMR results from the BBP architectural variants described, useful information about the mechanism of BBP-phospholipid interactions can be deduced. First, the hydrophobic norbornene backbone by itself may be too sterically hindered to insert into the membrane; and the hydrophobic PPO block may be useful to promote binding in some non-limiting instances. This result is consistent with the anchor-and-chain hypothesis; although in the bottlebrush architecture, the conformation of a PPO anchoring block has two distinct, non-limiting possibilities, which are illustrated in FIG. 7. In the “flagpole” conformation the backbone orients normal to the liposome surface, while the PPO block occupies a relatively large cylindrical volume within the bilayer. On the other hand, in the “in-plane” conformation the backbone lies largely in the bilayer plane and the side chains interdigitate with the lipids parallel to the lipid tails. Understanding whether either conformation is preferred can be useful for further engineering the molecular design, because polymer-lipid interactions are likely different between these conformations. For example and without limitation, in the flagpole conformation the lateral surface area covered by a single chain is related to the side chain length of the PEO block, and the insertion depth can be affected by the PPO block contour length. Meanwhile, for the in-plane conformation, the surface area coverage can be estimated by projecting a rectangle with dimensions proportional to the side chain and backbone lengths and the insertion depth by the side chain lengths of both blocks. Based on the side chain lengths and grafting density, BBPs are likely loose brushes in some non-limiting cases, so the side chains can be considered Gaussian coils while the backbone is extended. The core dimensions of BBP micelles can reflect behavior of a worm-like chain with a persistence length of ˜10 nm, which was higher than the 0.7 nm persistence length of linear polynorbornene.

TABLE 9A
Summary of micelle and bound populations

| Polymer | f_{micelle} [mol %]^{a} | f_{bound} [mol %]^{b} |
|---|---|---|
| L-E₉₃P₅₄E₉₃ (F127) | 0^{b} | 2.3 ± 0.9^{b} |
| B-E₁₀⁴³ P₅¹⁵ | 21 ± 9 | 7.3 ± 1.3 |
| B-E₂₁⁴³ P₁₁¹⁵ | 56 ± 23 | 0 |
| B-E₄₀¹⁰ P₇¹⁵ | 77 ± 11^{d} | 0 |
| B-E₄₀¹⁰ -s-P₇¹⁵ | 0 | 0 |
| B-E₁₁⁴³ P₆¹⁵ | 29 ± 13^{d} | 8.3 ± 4.1^{d} |
| B-E₁₂⁴³ -s-P₆¹⁵ | 5.1 ± 5.1^{d} | 0 |
| L-E₄₅₅ | 0^{c} | 0^{c} |
| B-E₁₅⁴³ | 0^{c} | 0^{c} |

^{a}Calculated via the constrained tri-exponential fitting procedure described in the methods section unless otherwise noted.
^{b}Unconstrained bi-exponential model, as the polymer-only control showed a single exponential decay.
^{c}Single exponential model applied. Error is the standard deviation of 3 independent replicates unless otherwise noted.
^{d}Error is the standard deviation of 2 independent replicates.

Without wishing to be limited by mechanism, an in-plane conformation may be more likely because it could enable the PPO side chains to intercalate between lipid molecules parallel to the acyl tails. In contrast, for the BBP to occupy the flagpole conformation, the polymer would have to overcome both van der Waals and hydrophobic forces to create the open volume in the lipid bilayer required for the PPO block to insert. Therefore, the in-plane conformation likely has a lower free energy than the flag-pole conformation. Additionally, the relationships between architectural parameters and membrane binding affinity are more consistent with the in-plane conformation. For example and without limitation, in the flagpole architecture, one would not expect the PEO side chain length to have as significant of an impact on binding ability as observed, e.g., that shortening the PEO block side chain length by a factor of ˜4 eliminates binding ability. Furthermore, this effect is consistent with the in-plane conformation because as side chain length is increased, the flexibility on the periphery of the brush could enable the PEO chains to intercalate into the bilayer, while shorter PEO chains may be too stiff and therefore would be excluded. Finally, if BBPs adopted the flagpole conformation, then when the contour length of the PPO block exceeds the bilayer thickness, the polymer would likely disrupt the bilayer. Cryo-TEM images of POPC liposomes incubated with B-E₁₆₈P⁴³P₄₃¹⁵, a BBP whose PPO block contour length is ˜6 times longer than the acyl region of the bilayer, showed no membrane perturbations or open vesicles. Although B-E₁₆₈⁴³P₄₃¹⁵ likely has very low membrane affinity, due to the relationship between Mₙ and f_{bound} described herein, liposome stability in its presence suggests that this molecule is less likely to assume a flagpole conformation. Nonetheless, regardless of whether the polymer adopts a flagpole, in-plane, or other conformation, the polymers herein can be assessed in a manner to determine whether such a conformation affects binding to liposomes.

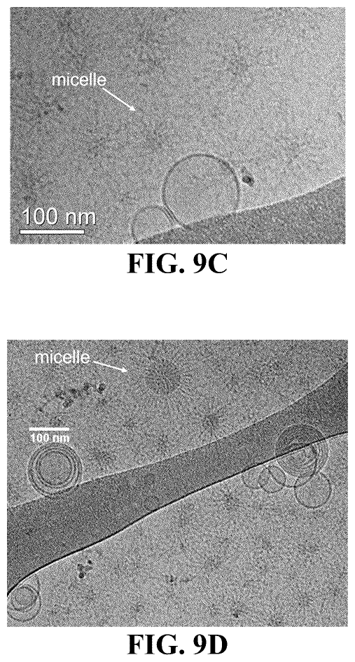

Direct experimental evidence of the conformation of membrane-bound BBPs is difficult to obtain due to the dynamic nature of phospholipid bilayers and the relatively low fraction of chains bound. PFG-NMR experiments were performed on liposomes treated with B-E₁₁⁴³P₆¹⁵ over a series of lipid concentrations to estimate the number of polymers bound to each liposome. Based on polymer characterization data, the surface area coverage for both conformations was calculated using simple geometrical approximations. For both conformations, a wide range of surface areal coverage (˜25%-100%) was observed. DLS experiments conducted at the same concentrations revealed that the Rₕ of the liposomes were independent of polymer treatment and within error of the neat liposome control. Because the flagpole conformation protrudes from the liposome surface by ˜10 nm while the in-plane conformation protrudes by only ˜3 nm, this result is consistent with the in-plane conformation. However, this analysis should be viewed cautiously because the Rₕ of polymer-treated liposomes cannot be described simply as the sum of the liposome radius and the contour length of the polymer, since it is unknown how an incompletely coated brush layer would impact diffusion. Details of this experiment and analysis are shown in FIG. 11A-D and as described in Example 10. In addition to experimental observations, molecular dynamics simulations may be conducted to assess the conformation of bound BBPs.

In both linear and bottlebrush poloxamers, the block architecture may be useful to promote binding in some non-limiting instances. Without wishing to be limited by the proposed mechanism, a hydrophobic PPO block could act as an anchor, and a PEO block could interact with the hydrophilic headgroups and the bound water layer. However, based on the effect of PEO side chain length on BBP-liposome binding, the PEO block may also play a role in providing additional interpenetration with phospholipids. Without wishing to be limited by theory, the enhanced membrane affinity of B-E₁₀⁴³P₅¹⁵ relative to an analogous linear poloxamer may be attributable to an enhanced interdigitation effect.

All liposome models are distant abstractions from a living membrane, but they are useful because their simplicity allows one to ask targeted questions by specifying the desired bilayer constituents. Single component POPC liposomes were tested to compare membrane binding ability of bottlebrush and linear poloxamers and to explore the effect of bottlebrush architectural parameters. Lipid head and tail-group identities can affect interactions between linear poloxamers and lipid bilayers. For instance, linear poloxamers can have a higher affinity for POPG liposomes than POPC liposomes, likely due to hydrogen bonding between the ether groups of the polymer and the alcohol of the POPG. BBPs may also have a higher affinity towards a POPG bilayer (or any lipid bilayer with hydrogen bond donors in the headgroup region) than a POPC bilayer since BBPs contain many ether groups in close proximity to the lipid bilayer. Additionally, linear poloxamers typically have a higher affinity towards bilayers composed of DOPC than POPC because the additional unsaturated tail in DOPC reduces the bending modulus, facilitating polymer insertion. Without wishing to be limited by mechanism or theory, this effect is likely independent of polymer architecture, and therefore BBPs could show a similar relationship between binding and lipid tail unsaturation.

Molecular structure was systematically varied to explore the effects of several bottlebrush polymer architectural parameters including side chain length, overall molecular weight, and monomer distribution (statistical versus block). The membrane binding ability of a BBP can be modified, for example and without limitation, by adjusting the side chain length and backbone degree of polymerization. In one non-limiting case, a high affinity BBP had approximately a three times higher membrane affinity than an analogous linear poloxamer, despite a more rigid and crowded architecture. The hydrophobic backbone of the bottlebrush likely does not participate significantly in direct membrane binding. Without wishing to be limited by theory, increased binding may be attributed to the ability for side chains to interdigitate between neighboring phospholipids. These results can be used to tune membrane affinity of amphiphilic macromolecules by providing mechanistic insight about which parts of the molecule drive binding.

Additionally, as described herein, BBPs can protect cell membranes from osmotic stress. The tested bottlebrush polymers ranged in total molecular weight from 26 kDa to 260 kDa, and they maintained protective ability throughout this range. Linear block copolymer amphiphiles have only demonstrated protection efficacy up to ˜20-30 kDa. The extension in molecular weight of protective BBPs may be useful, as renal clearance can depend on molecule size. Finally, unlike liposome binding, cell membrane protection was provided by different polymeric structures, including bottlebrush PEO homopolymers and statistical copolymers with PEO and PPO side chains, which outperformed L-E₈₇P₃₁E₈₇ (P188) in an osmotic stress assay on a molar basis. This is another example of low affinity polymers being protective in vitro and re-emphasizes that there may be differences between studies of polymer-liposome binding and in vitro cellular protection.

### Example 10

### Conformation of Bound BBPs Via PFG-NMR and DLS

To obtain experimental insight into the conformation of bound BBPs, PFG-NMR and dynamic light scattering experiments were performed over a series of POPC concentrations to determine if the Rₕ of the liposomes is a function of polymer surface coverage. If the flagpole conformation exists, then a high surface coverage a BBP with a PEO block contour length of ˜10 nm could impact the Rₕ of the liposomes in an observable way.

B-E₁₁⁴³P₆¹⁵ was employed due to its membrane affinity. PFG-NMR binding assays were performed over a range of liposome concentrations. The decay curves are shown in FIG. 11B, and binding was detected in all cases except for the polymer-only control. As the liposome concentration decreases, there are fewer binding sites and, thus, the amount of polymer bound per liposome was expected to increase. The number of polymers bound to each liposome can be calculated based on the PFG-NMR data, lipid and polymer concentrations, the fraction of the liposome surface area that is exposed to the solution (estimated via cryo-TEM), and an estimate of the number of lipid molecules per liposome. Then, the surface area per chain in the flagpole and in-plane conformations can be estimated from BBP characterization data. Finally, the fraction of the liposome surface area covered by polymer was calculated for both proposed conformations, and the results are shown in FIG. 11C.

Regarding FIG. 11C, the number of polymers bound to each liposome (N_{poly}) was calculated by Eq. 3A:

$${N}_{poly} = \frac{c}{{M}_{n}} * \frac{{f}_{bound}}{{n}_{lipid} f} * {N}_{tot} , \qquad ( 3 A )$$

where c is the polymer concentration (14 μM), Mₙ is the number average molecular weight, f_{bound} is the mol fraction of chains bound to liposomes which was estimated by the tri-exponential model, n_{lipid} is the concentration of lipid (875 μM), f is the fraction of liposome surface area that is on the exterior bilayer and thus available for polymer binding (estimated via cryo-TEM in FIG. 9), and Nₜₒₜ is the number of lipids per liposome. Nₜₒₜ was estimated by Eq. 3B:

$${N}_{tot} = \frac{4 π {R}_{lipo}^{2}}{A} + \frac{4 {π ( {R}_{lipo} - t )}^{2}}{A} , \qquad ( 3 B )$$

where Rₗᵢₚₒ is the hydrodynamic radius of the liposomes measured by DLS, A is the surface area per lipid (0.683 nm²), and t is the bilayer thickness (5.2 nm). This calculation to calculate the fraction of the liposome surface that is covered by polymers. Knowing the chain dimensions and the number of polymers bound to the surface, the surface area coverage for the two hypothesized conformations can be estimated. For the flagpole conformation, the PEO side chains were approximated as Gaussian coils, and projected the circle with radius of 2R_{g, PEO} side chains onto the liposome surface. Thus, the area occupied by a single chain in the flagpole conformation (A_{flagpole}) was estimated as follows:

$${A}_{flagpole} = 4 π {N}_{sc , PEO} {b}_{PEO}^{2} / 6 , \qquad ( 3 C )$$

where N_{sc, PEO} is the side chain degree of polymerization for the PEO block and b_{PEO} is the statistical segment length of PEO (0.6 nm). For the in-plane conformation, each bound polymer was approximated by adjoined rectangles and semi-circles. In this model, the rectangle represents the cross section of the cylindrical bottlebrush, and the semi-circle the terminal side chain, which can protrude in the axial direction. Rectangle dimensions were estimated by the contour length of the backbone and 4R_{g, sc}, and the semicircle had a radius of 2R_{g, sc} described as follows:

$${A}_{in - plane} = 4 {{L}_{bb , PPO} ( \frac{{N}_{sc , PPO} {b}_{PPO}^{2}}{6} )}^{\frac{1}{2}} + \frac{1}{2} π ( 4 \frac{{N}_{sc , PPO} {b}_{PPO}^{2}}{6} ) 4 {{L}_{bb , PEO} ( \frac{{N}_{sc , PEO} {b}_{PEO}^{2}}{6} )}^{\frac{1}{2}} + \frac{1}{2} π ( 4 \frac{{N}_{sc , PEO} {b}_{PEO}^{2}}{6} ) , \qquad ( 3 D )$$

where L_{bb} is the contour length, N_{sc} is the side chain degree of polymerization, and b is the statistical segment length for the given block (PPO or PEO block). Now, the fraction of the liposome surface (θₓ) that is covered by the BBP in each conformation was calculated as follows:

$${θ}_{x} = \frac{{N}_{poly} {A}_{x}}{4 π {R}_{lipo}^{2}} , \qquad ( 3 E )$$

where Aₓ represents the area occupied by a chain in the conformation of interest. FIG. 11C shows the relationship between the lipid concentration and surface area coverage for both conformations.

If BBPs adopt the in-plane conformation, then the surface area coverage ranges from complete coverage, 144±39%, to only 30±4% at the lowest and highest concentrations of liposomes, respectively. If the flagpole conformation predominates, then the surface area coverage ranges from 104±28% to only 22±3%. Thus, for both conformations a wide range of surface area coverages was observed. Multi-angle DLS results for POPC liposomes incubated with B-E₁₁⁴³P₆¹⁵ at the same three POPC concentrations and a neat liposome control are shown in FIG. 11D. The slopes of the I′ vs. q² relationships are identical within error, indicating that polymer incubation does not change the Rₕ of the liposomes. Therefore, the Rₕ of the liposomes is likely independent of polymer surface coverage under these tested conditions. This result is consistent with the in-plane conformation; however, the following should also be considered: (i) the Rₕ of polymer treated liposomes cannot be described simply as the sum of the liposome radius and the contour length of the polymer since it is not clear how much an incompletely coated brush layer would impact diffusion, and (ii) the difference in polymer dimensions normal to the bilayer between the two conformations is relatively small for B-E₁₁⁴³p₆¹⁵ (˜10 nm for flagpole vs. ˜3 nm for the in-plane). Due to these experimental challenges, the dynamic nature of phospholipid bilayers, and the low fraction of polymer chains that bind to the bilayer, further experiments and/or simulations may be useful ascertain conformation of bound polymers.

Note, the radius of the liposomes used in the PFG-NMR experiment was twice that of those used in the DLS experiment. The larger liposomes help discriminate between micelles and liposome-bound polymer in the PFG-NMR experiment by increasing the difference in size of these two states. As the curvature of the liposome increases, binding can increase. Thus, the fraction of surface area coverage shown in FIG. 11C is likely an underestimate of what is occurring in the DLS experiment. The smaller pore size was chosen in the DLS experiment to increase the % change in radius expected if the flagpole conformation persists.

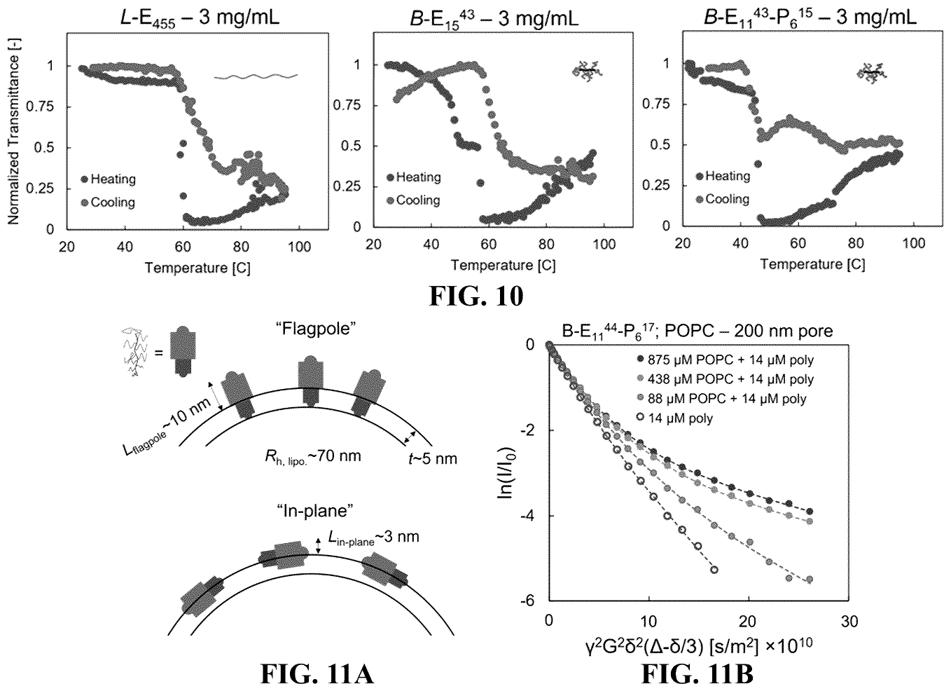

To further assess the bound BBP conformation, DLS experiments were repeated (as described herein) with B-E₁₆₈⁴³P₄₃¹⁵, which has a contour length of roughly 120 nm which is ten times longer than that of B-E₁₁⁴³P₆¹⁵. Thus, without wishing to be limited by mechanism, if this polymer adopts the flagpole conformation, it may protrude much further from the liposome surface and be more likely to affect diffusion. Due to the high Mₙ of B-E₁₆₈⁴³P₄₃¹⁵, the polymer concentration was reduced to minimize scattering from unimers. FIG. 12A shows the multi-angle DLS data for three conditions of POPC liposomes treated with B-E₁₆₈⁴³P₄₃¹⁵, and the Rₕ of all conditions are within error. Thus, there is no evidence of a flagpole conformation under these tested conditions. Furthermore, REPES analysis shown in FIG. 12B shows that B-E₁₆₈⁴³P₄₃¹⁵ treatment does not change the liposome size distribution. This corroborates the TEM result that B-E₁₆₈⁴³P₄₃¹⁵ is not disruptive to lipid bilayers.

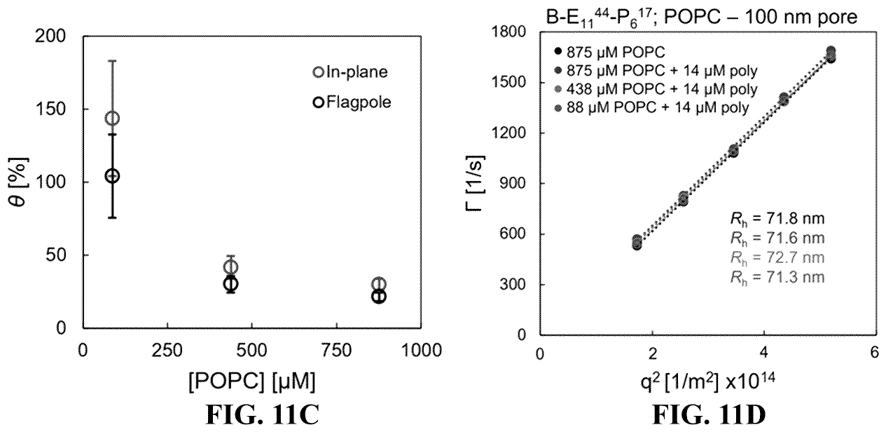

Extending the backbone of the hydrophilic block can cause a shape change of the molecule (see, e.g., FIG. 13A). When the side chain length is longer than the backbone, the molecule can occupy a roughly spherical conformation, and this is called the star-like regime. When the backbone is extended and becomes longer than the side chains, the molecule can be characterized as being cylindrical, which can be considered a non-limiting bottlebrush regime.

When the backbone was extended past the star-to-brush transition, the size of micelles (Rₕ˜20 nm) approaches that of the liposomes (Rₕ˜30 nm). Therefore, the PFG-NMR binding assay is less likely to distinguish polymers between these two states. In other words, the overlap between the polymer-only control and the polymer+liposome samples for B-E₅₇⁴³P₅¹⁵ in FIG. 13C is not necessarily evidence that no polymer binds because chains in the micelle state and liposome state are less distinguishable. However, given the relationship between molecular weight and binding, no detectable binding occurred for B-E₅₇⁴³P₇¹⁵.

The osmotic stress protection assay data in FIG. 13D demonstrate that the protective ability is not lost when the PEO block backbone is extended. The polymer with the longest PEO backbone (B-E₁₁₇⁴³P₇¹⁵) is significantly more protective than the shortest backbone polymer at a polymer concentration of 0.2 μM. In addition, the protective efficacy of B-E₁₁₇⁴³P₇¹⁵ and for B-E₅₇⁴³P₇¹⁵ decreases relative to the linear controls and B-E₁₀⁴³-P₇¹⁵ at higher concentrations. As there is little correlation between binding affinity in the PFG-NMR assay and protection in the osmotic stress assay, a decrease in membrane affinity is unlikely the sole explanation, and other factors may be implicated.

FIG. 13E shows the same data as in FIG. 13D but in mass units. When the data are plotted this way, the higher molecular weight BBPs are less protective than the smaller ones. This may suggest that distributing PEO/PPO mass among more chains could be beneficial because it could lead to more binding sites across the membrane surface. In some cases, this could allow the polymer to manipulate the mechanical properties of the membrane in more locations and therefore more uniformly.

### Example 11

### Synthesis and Micellization of Bottlebrush Poloxamers

Synthesis of BBPs can be challenging. Controlled radical polymerization can produce grafted PPO polymers; however, these methods can result in low monomer conversion or low grafting density to maintain modest dispersities. Described herein are synthetic routes to BBPs that circumvents these challenges and gives more control over parameters, such as graft length and graft density. Micellization behavior of a set of BBPs was examined to understand the relationship between the apparent critical micellization concentration (CMCₐ) and the total molecular weight. The resulting scaling relationship provides a non-limiting comparison of the thermodynamic drive towards micellization between the bottlebrush and the chemically analogous linear architectures.

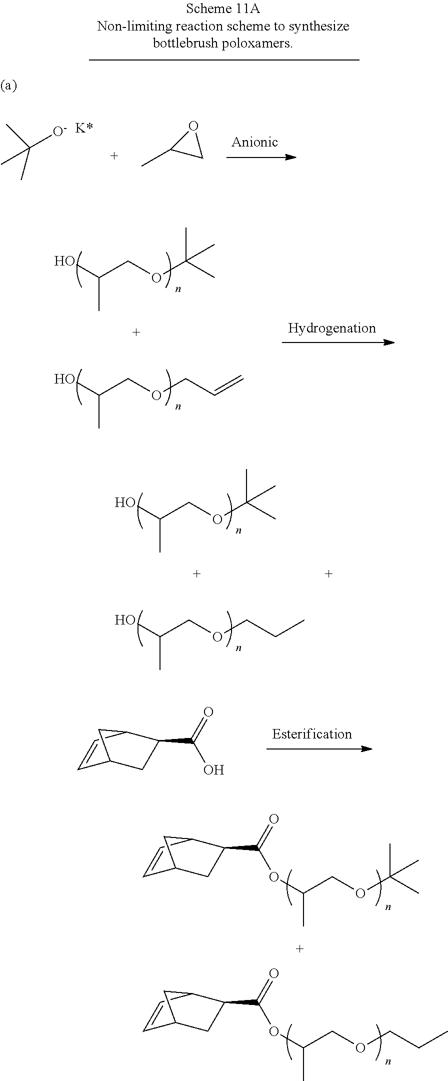

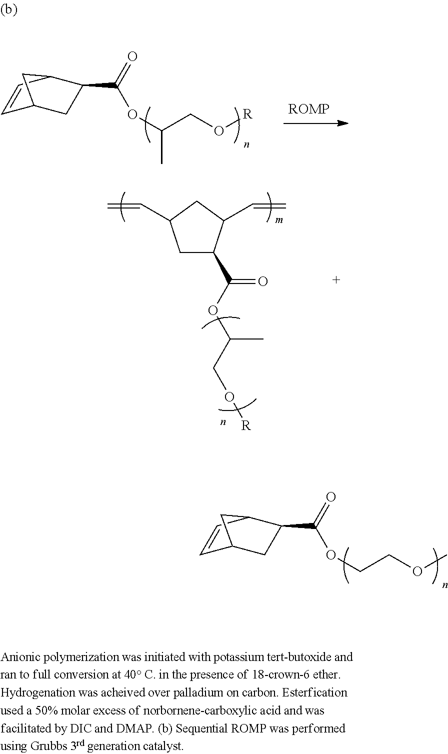

A non-limiting synthetic route is shown in Scheme 11A. Living anionic polymerization followed by chain end modification was used to make norbornene (NB)-functionalized macromonomers (MMs), which were then polymerized via ring opening metathesis polymerization (ROMP). ROMP has successfully been combined with living polymerization strategies (e.g., such as anionic, RAFT, and ATRP) and is compatible with PEO. This strategy has several non-limiting advantages. First, anionic polymerization can yield low dispersity PEO and PPO polymers of a targeted molecular weight with well-defined α-chain ends. Second, ROMP of NB macromonomers can reach >99% conversion within minutes. Third, ROMP can avoid chain transfer/coupling side reactions that are often observed during radical polymerization, to which PPO macromonomers can be vulnerable due to the abstractable proton on the tertiary carbon site of every repeat unit. Together, these advantages could allow for, in some non-limiting instances, high-throughput synthesis of high molecular weight, high grafting density, and/or low dispersity BBPs with control over graft length and graft end group. Regarding BBP nomenclature, BB-E_{q,p}-b-BB-P_{n,m} can be used, in which “BB” indicates bottlebrush, “E” and “P” indicate PEO and PPO, respectively, and the subscripts are number average degrees of polymerization in reference to Scheme 11A.

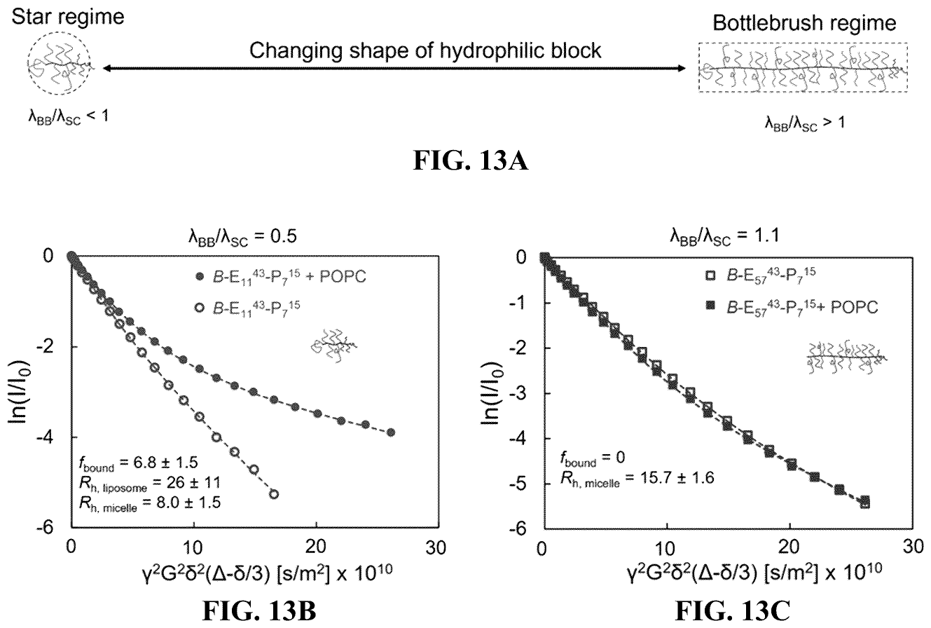

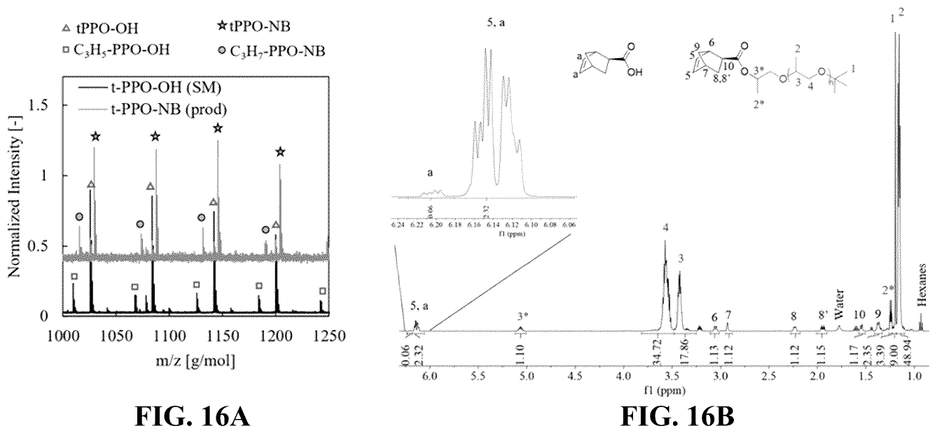

The black (lower) trace in FIG. 15 shows matrix-assisted laser desorption/ionization mass spectrometry (MALDI) data of the product PPO from anionic polymerization. Notably, there are two populations with peak spacings of 58 g/mol, indicating both are PPO. The more intense family of peaks is the product with a tert-butyl α-chain end and an alcohol ω-chain end (t-PPO-OH). The family labelled with open squares is an impurity with an alkene α-chain end, which likely arises from deprotonation of PPO methyl groups during polymerization. This assessment was confirmed by ¹H nuclear magnetic resonance (NMR) spectroscopy. This side reaction can be limited by using 18-crown-6 ether, but roughly 7 mol % (from NMR) of the chains can have this impurity. This alkene impurity is likely to be susceptible to cross-metathesis during ROMP, leading to high dispersity polymers with limited control over molecular weight (see FIG. 19). Thus, a post-polymerization hydrogenation was conducted to eliminate this alkene impurity. The MALDI data in FIG. 15 show a 2 g/mol increase in molecular weight for each of the peaks identified in the alkene chain end population, open squares to open circles, which indicates that this chain end modification was successful without changing the molecular weight distribution.

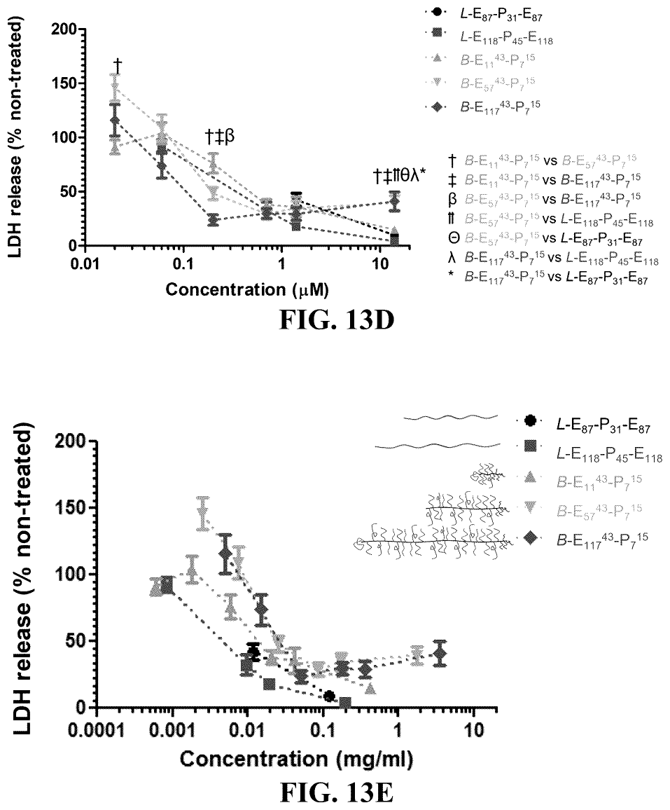

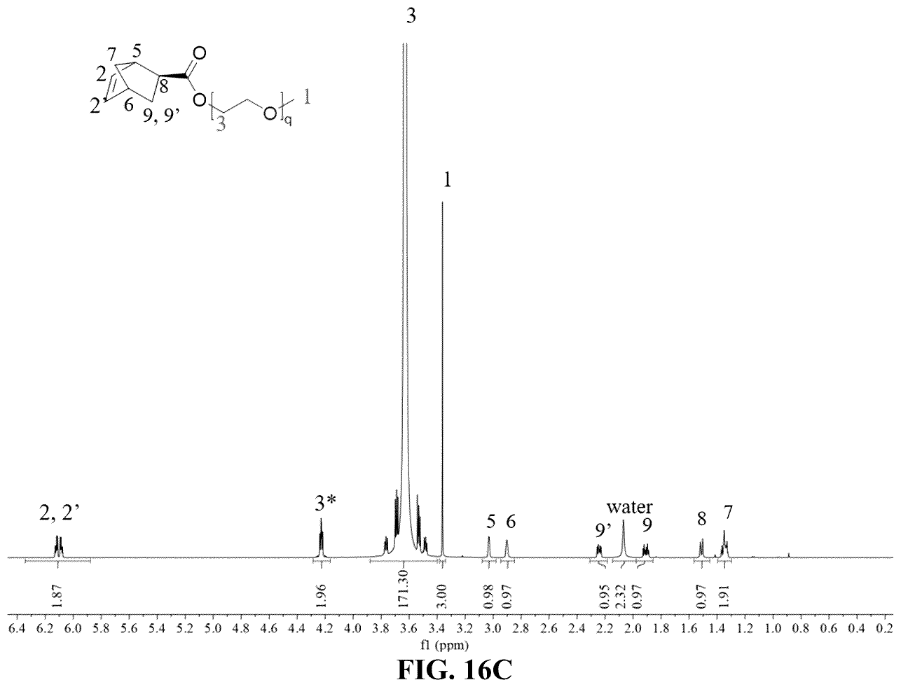

The final step of MM preparation was to convert the ω-alcohol to a NB-ester through a modified protocol for the PPO chemistry. FIG. 16A shows a close-up of the MALDI spectrum to highlight the peak shifting after chain end modification. The open triangle population, present as the majority in the starting material (black, lower spectrum) and a minority in the product (gray, upper spectrum), is t-PPO-OH. After esterification, this population shifted by +4 g/mol in m/z (stars), indicating successful addition of a NB unit. Note that adjacent peaks do not have the same degree of polymerization, thus the expected peak shift after an end group modification is the difference in molecular weight of the end groups minus an integer multiple of the repeat unit molecular weight. A similar shift, +6 g/mol, occurred between the square and circle populations, which correspond to the alkene-PPO-OH and alkane-PPO-NB, respectively. Note the lower (black) trace is from the material prior to hydrogenation, while the upper (gray) trace is post-hydrogenation and esterification. FIG. 20 shows size exclusion chromatography (SEC) traces of the PPO post anionic polymerization and the functionalized MM, and the molecular weight distributions are comparable. ¹H NMR spectroscopy was used to assess the purity of the resulting polymer and to corroborate successful chain end modification. The peak shape and integration of the alkene multiplet at 6.09-6.24 ppm (FIG. 16B) suggests that 95 mol % of the NB is on a polymer chain end. Excess NB-carboxylic acid can be used, in some instances, to achieve high conversion. Of note, such excess may be difficult to remove due to its similar hydrophobicity to PPO and the small molecular weight of the PPO. A certain level of purity may be acceptable, and some incorporation of NB-carboxylic acid may be present (e.g., as a diluent) in the polymer backbone. When accounting for the small molecule (˜5 mol %) and the alkane ω-end group (˜7 mol %) impurities in the analysis of the integral values of the NB peaks (6, 7, 8), which were referenced to the t-butyl peak (1), quantitative conversion of alcohol to NB at the ω-end groups was achieved. A similar protocol was used to synthesize a NB-functionalized MM from a commercial methyl ether PEO (Mₙ=1970 g/mol, Ð=1.04) (FIG. 16C).

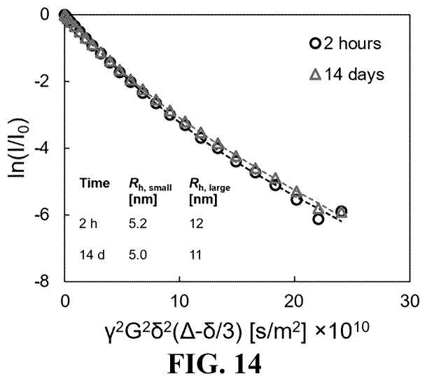

After synthesis of MMs, sequential ROMP afforded a grafted polynorbornene diblock polymer, where one block has PEO side chains and the other PPO side chains. To our knowledge, this is the first report of a bottlebrush PEO-b-PPO polymer. The representative SEC traces in FIG. 17 show that both the PPO block and the diblock have narrow dispersities, achieve the target molecular weight, and shift systematically to shorter retention times for higher Mₙ. The ¹H NMR spectrum shows complete conversion of the monomeric NB alkene peaks to backbone alkene peaks, confirming that the reaction achieved full conversion of monomer. To our knowledge, this is the first report of a grafted PPO polymer where the macromonomer was fully consumed, making synthesis of multi-blocks possible.

To understand the micellization behavior of these BBPs, three polymers were synthesized with constant side chain lengths and similar compositions (70-80 wt % PEO), with total molecular weights ranging from 26 kDa to 394 kDa. All materials had narrow molecular weight distributions (Ð<1.15), and the characterization data are summarized in Table 11A. Micelle solutions were prepared via direct dissolution in aqueous buffer and were equilibrated at 37° C. for at least 24 hours. The CMCₐ and hydrodynamic radii (Rₕ) were determined via dynamic light scattering (DLS). DLS measures the diffusive relaxation of concentration fluctuations in dilute solution, which yields the particle size distribution by analyzing the correlation function via cumulant analysis or inverse Laplace techniques such as regularized positive exponential sum.

TABLE 11A
Materials and micellization data summary

| | | | | | | | R_{core} | R_{core} |
|---|---|---|---|---|---|---|---|---|
| | Mₙ | | wt % | wt % | CMCa | Rₕ | [nm]^{d} | [nm]^{e} |
| Polymer | [g/mol]^{a} | Ð^{a} | PEO^{a} | NB^{a} | [mg/mL]^{b} | [nm]^{c} | (calc) | (expt) |
| E₄₅₀ (PEO-20k) | 20000 | 1.10 | 100 | 0 | N/A | | | |
| E₇₆-b-P₂₉-b-E₇₆ (F68)^{f} | 8400^{f} | | 80^{f} | 0 | 70 ± 10^{f} | | | |
| E₁₀₃-b-P₃₉-b-E₁₀₃ (F88)^{f} | 11400^{f} | | 80^{f} | 0 | 6 ± 0.9^{f} | | | |
| E₁₃₂-b-P₅₀-b-E₁₃₂ (F108)^{f} | 14600^{f} | | 80^{f} | 0 | 0.4 ± 0.1^{f} | | | |
| BB-E_{45, 10}-b-BB-P_{15, 5} | 26200 | 1.07 | 72 | 8 | 8 ± 3 | 17 ± 4 | 5 | 6 ± 1 |
| BB-E_{45, 20}-b-BB-P_{15, 10} | 55000 | 1.10 | 74 | 8 | 2 ± 0.5 | 25 | 8 | |
| BB-E_{45, 160}-b-BB-P_{15, 43} | 394000 | 1.15 | 80 | 7 | 0.4 ± 0.1 | 150 | 30 | 15 ± 8 |

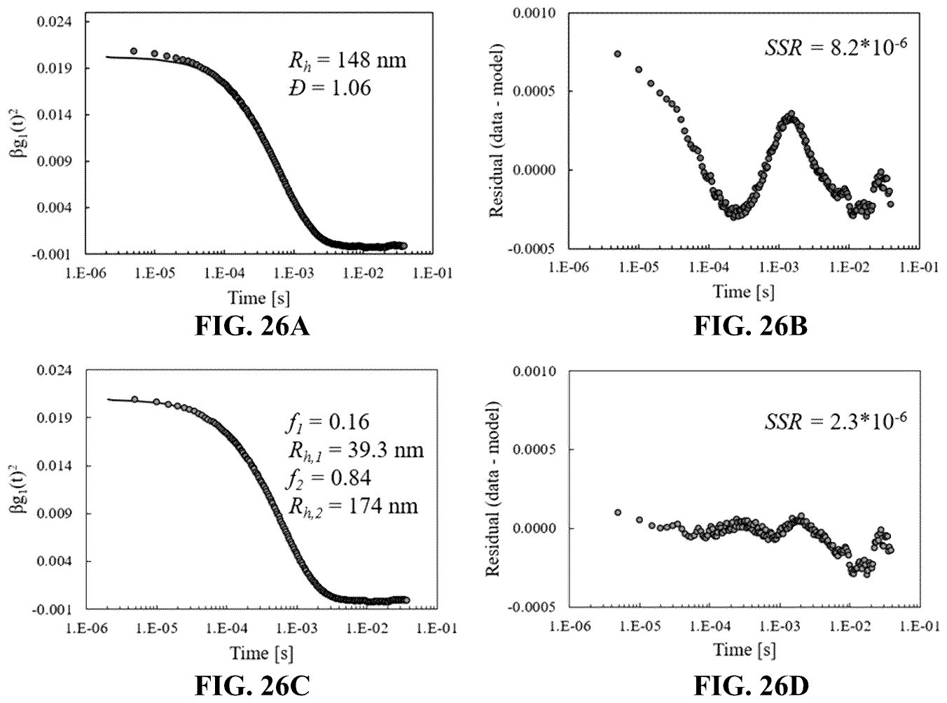

^{a}SEC with multi-angle light scattering detection and Zimm analysis
^{b}Determined by DLS
^{c}DLS data reported for 10 mg/mL solution using the second cumulant model. Error is the standard deviation of three independent replicates.
^{d}Model described in FIG. 29: sum of core forming block's backbone contour length and twice the radius of gyration of its side chains
^{e}Determined by cryo-TEM
^{f}Data from Alexandridis P et al., Macromolecules 1994; 27(9): 2414-2425 measured via fluorescent dye method

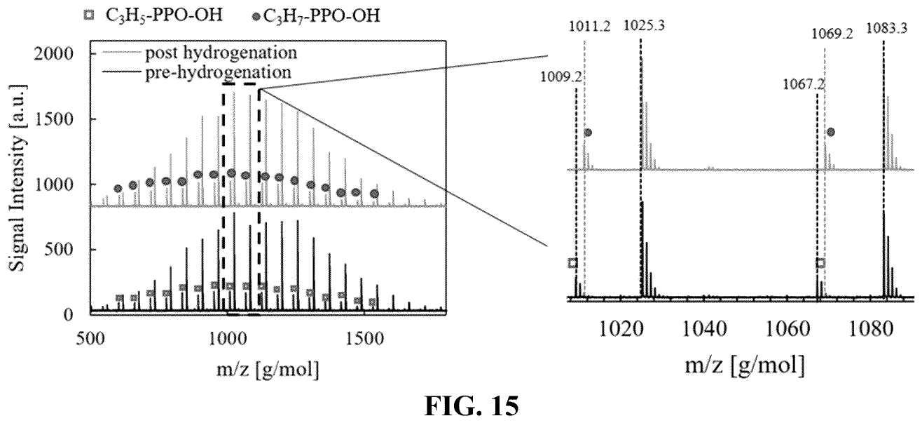

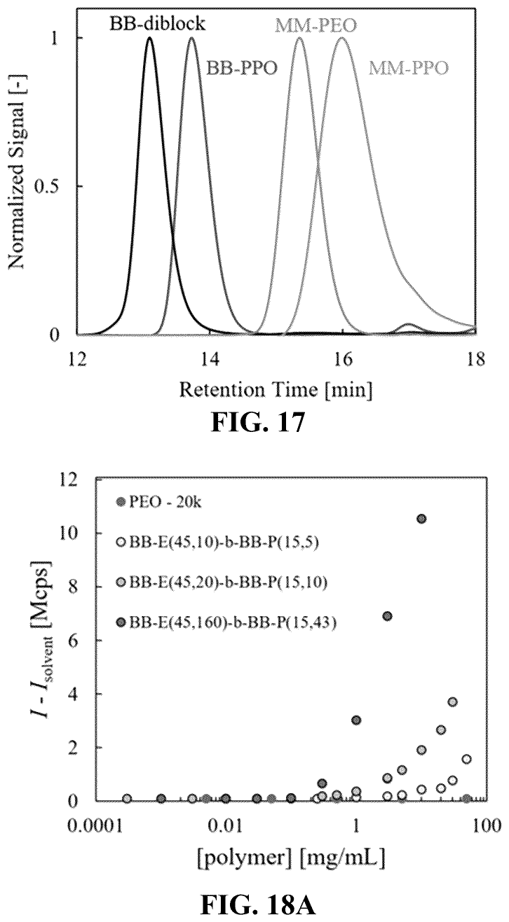

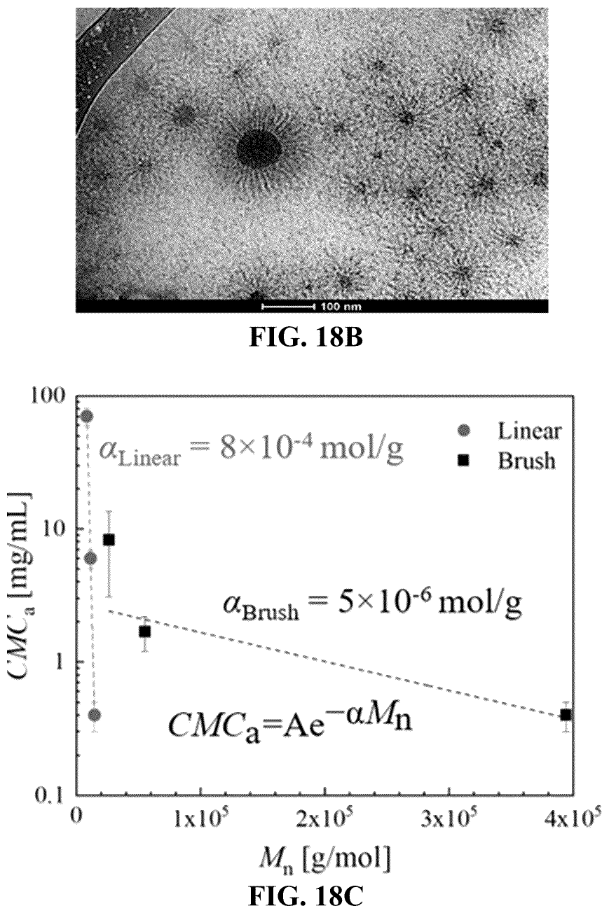

To estimate the CMCₐ, the excess scattering intensity (Iₑₓ=I−Iₛₒₗᵥₑₙₜ) was analyzed as a function of polymer concentration. Because Iₑₓ scales with the product of the number of scatterers and the size of the scatters, the concentration at which Iₑₓ increases relative to the control (linear PEO homopolymer with Mₙ=20 kDa) indicates likely aggregation (FIG. 18A). To ensure that the aggregates are micelles, cryo-TEM was performed on the smallest and largest BBPs. A representative micrograph of the largest polymer is shown in FIG. 18B, which demonstrates a core-corona structure. Micrographs of the smallest polymer show a core, although the corona block is too short to be observed. Additional micrographs are shown in FIG. 21A-D and FIG. 22A-C.

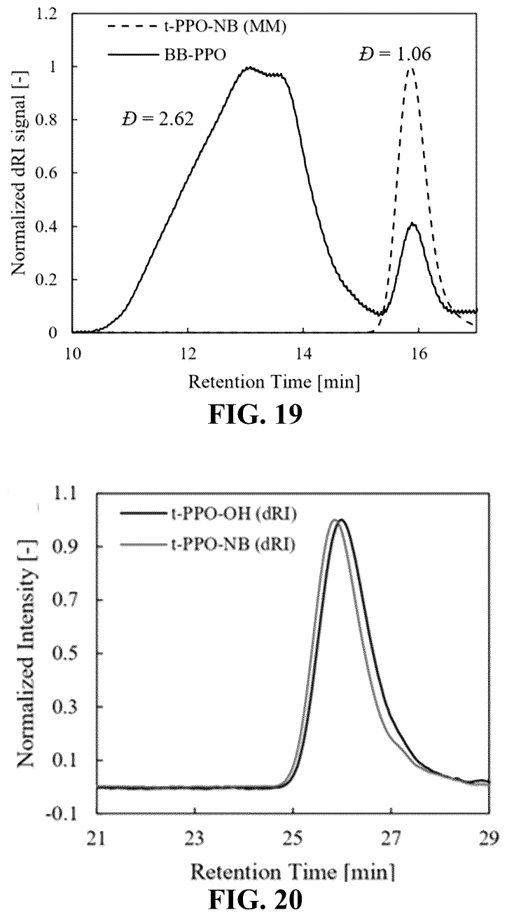

On qualitative inspection of FIG. 18A, as Mₙ increases, the CMCₐ decreases. Quantitative analysis can be challenging because micellization is not a phase transition; therefore, a precise CMCₐ can be difficult to extract and can be sensitive to the method employed. A representative CMCₐ was taken as the intersection of two logarithmic fits: one at low polymer concentrations, and one at high polymer concentrations, representing the free chain and micelle regimes, respectively. The error is estimated as the difference in the CMCₐ when the number of data points in the micelle regime is increased by 1. To ensure these CMCₐ estimates are representative, DLS data were fit to a closed association model, which includes the CMCₐ as a fitting parameter (FIG. 23A-B). Consistent trends were observed with both methods.

In brief, a model was applied to define the apparent critical micellization concentration CMCₐ; thus, removing all human decision making in the cut-off between the free-chain and micelle regimes. From light scattering theory, the excess scattering intensity depends on the product of the size and number of scatterers. Thus, for a mixture of unimers and micelles, the excess scattering intensity is given by Eq. 5.1:

$${I}_{ex} = A [ unimers ] + B [ micelles ] . \qquad ( 5.1 )$$

The closed association model asserts that for a solution at a concentration above the CMCₐ, there exists an equilibrium between unimers at a concentration equal to the CMCₐ and micelles of a constant number of chains, N_{agg}. This is expressed by the expressions shown below:

$${N}_{agg} unimers ↔ micelle and \qquad ( 5.2 )$$

$$[ unimers ] = \{ \begin{matrix}[ polymer ] , & [ polymer ] < {CMC}_{a} \\ {CMC}_{a} , & [ polymer ] \geq {CMC}_{a}\end{matrix} . \qquad ( 5.3 )$$

From a mol balance on the system, the concentration of micelles can be expressed as a function of the total concentration of polymer, chosen by the experimenter, the concentration of unimers, and the aggregation number:

$$[ micelles ] = \frac{[ polymer ] - [ unimer ]}{{N}_{agg}} . \qquad ( 5.4 )$$

Now, Eq. 5.1 can be expressed with a conditional expression using only experimental quantities, [polymer], and model parameters (CMCₐ, N_{agg}, A, and B):

$${I}_{ex} = \{ \begin{matrix}A [ polymer ] , & [ polymer ] < {CMC}_{a} \\ A \times {CMC}_{a} + B \times \frac{[ polymer ] - {CMC}_{a}}{{N}_{agg}} , & [ polymer ] \geq {CMC}_{a}\end{matrix} . \qquad ( 5.5 )$$

The relationship between Mₙ and CMCₐ for 80 wt % PEO linear poloxamers and 72-80 wt % PEO BBPs are shown in FIG. 18C. The free energy of a micelle is the sum of the core-corona interfacial energy, the energy of chain deformation, and the entropy of mixing between solvent and corona units. The closed association model asserts that, above the CMCₐ, there is an equilibrium between unimers ([unimers]=CMCₐ) and micelles of a constant size. Minimization of the total free energy of the system (micelles, unimers, and solvent) can yield an exponential scaling between the CMCₐ and Mₙ. Therefore, the data were fit to an exponential form as shown in Eq. 6:

$${CMC}_{a} = A \times {e}^{- α \times {M}_{n}} . \qquad ( 6 )$$

The coefficient in the exponent of the fit (slope in FIG. 18C) is two orders of magnitude smaller in the bottlebrush case than in the linear case. Because α˜χ, the Flory-Huggins interaction parameter, the reduced scaling coefficient suggests a decreased degree of solvent-core incompatibility, and therefore reduced thermodynamic driving force for micellization, in BBPs than in linear poloxamers. Without wishing to be limited by theory, one explanation for this is that the PPO units near the hydrophobic NB backbone are relatively dehydrated in the unimer state. Thus, some of the entropic gain in the solvent upon dehydrating PPO in going from unimer to micelle can be lost. Additionally, a BBP with Mₙ˜400 kDa has a comparable CMCₐ to a linear poloxamer with Mₙ˜14 kDa, which is consistent with molecular dynamics results. This finding can be of practical significance because, in BBPs, the CMCₐ is less sensitive to molecular weight, enabling synthesis of vastly higher Mₙ materials that can exist as unimers in aqueous solutions.

This reduction of α for BBPs, as compared to linear poloxamers, cannot be explained by the hydrophobic backbone alone. If the added hydrophobicity of the backbone were a significant contributor to micellization, an opposite effect could be observed, in which BBPs would form micelles at lower concentrations compared to linear poloxamers due to the added hydrophobicity. Rather, the backbone is likely shielded from the aqueous environment by the side chains to reduce the enthalpic contribution of water-backbone interactions. Similarly, the 5 mol % carboxylic acid impurity along the backbone is presumably not fully exposed to the aqueous environment and likely does not significantly impact the CMCₐ. Also, the effect of the alkane α-chain end impurity on micellization is assumed to be negligible because it represents less than 0.3 wt % of the hydrophobic block and has a minimal difference in hydrophobicity compared to either the tert-butyl end group or the PPO repeat unit.

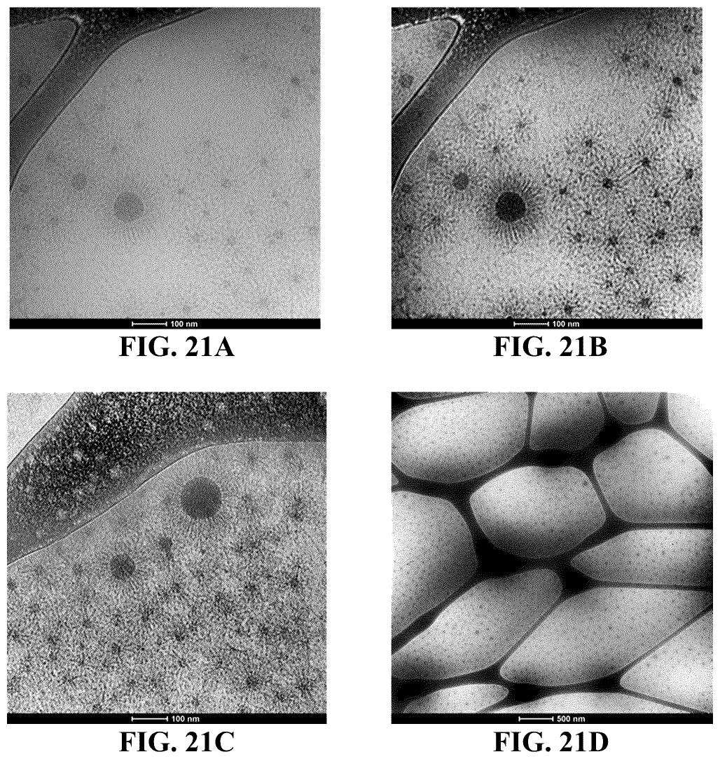

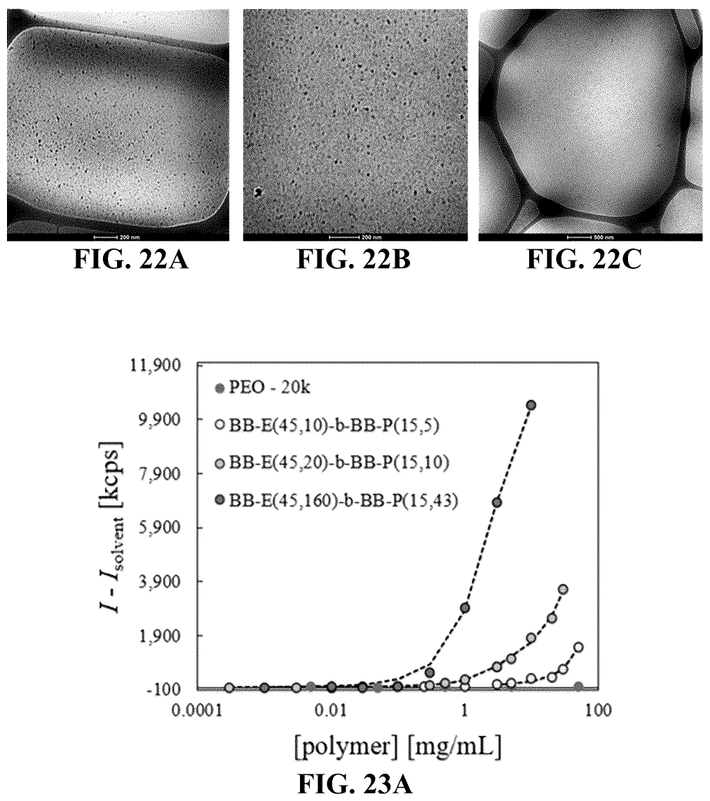

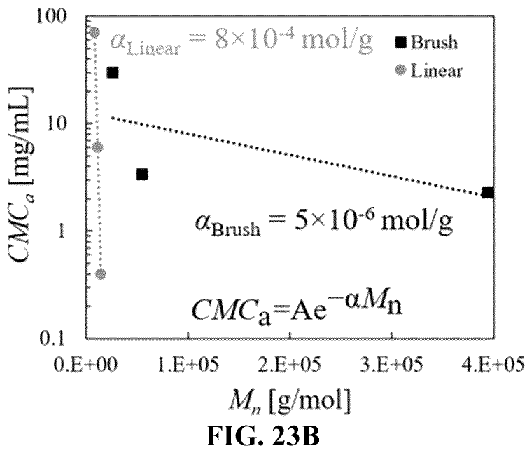

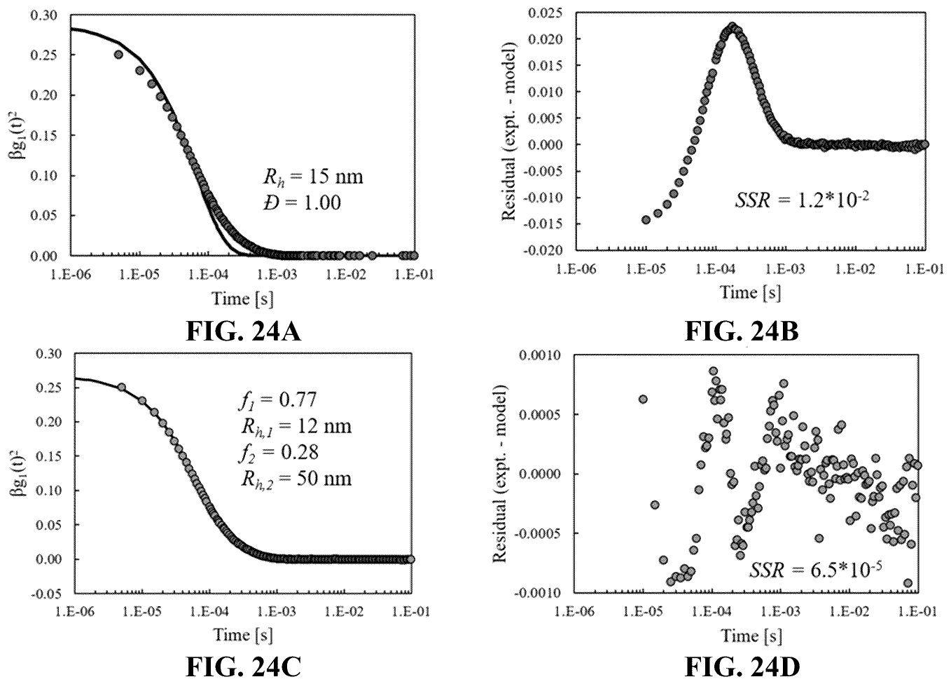

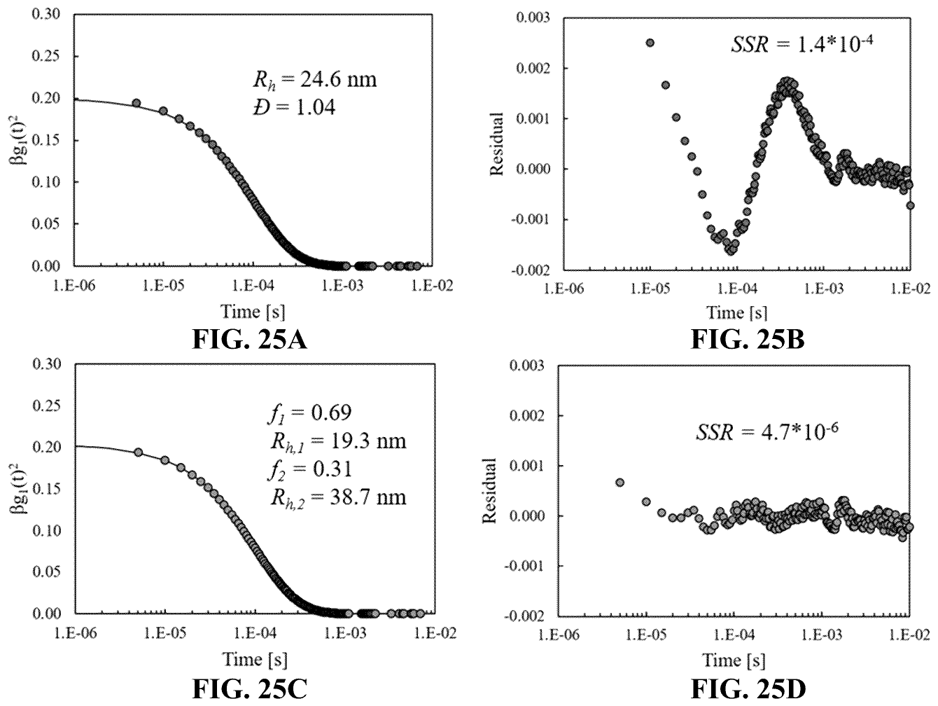

Analysis of the correlation functions via the second cumulant model (Eq. 7.1) shows that Rn increases approximately linearly with molecular weight, suggesting that the chains adopt an extended conformation in the micelles. Additionally, for every micellar BBP solution a bi-exponential model (Eq. 7.2), accounting for two relaxation rates, yields a superior fit than a single exponential model (FIG. 24A-D, FIG. 25A-D, and FIG. 26A-D). Multi-angle DLS on a 3 mg/mL solution of the highest Mₙ polymer confirmed that both relaxation modes are diffusive and have Rₕ values larger than expected for a unimer, suggesting existence of a bimodal micelle size distribution (FIG. 27A-B). The TEM micrograph in FIG. 18B also suggests a bimodal population of spherical micelles. This could be due to the direct dissolution method not allowing the micelle size distribution to fully equilibrate; however, the micelle size distribution does not change when annealed at 37° C. for 35 days (FIG. 28A-B). This suggests the micelles have reached a steady state. Without wishing to be limited by mechanism or theory, side chain length asymmetry of the two blocks can impact micelle morphology, aggregation number, and surface roughness due to packing frustration at the core-corona interface. Furthermore, the additional free energy contribution of side chain crowding near the core-corona interface could create at least two local minima in free energy-micelle size space that are separated by a significant energy barrier; thus leading to a bimodal micelle size distribution.

To estimate the micelle core radius (R_{core}), the core-forming block was modelled as a cylinder plus a hemisphere, where the cylinder length equals the contour length of the backbone and the hemisphere radius is twice the radius of gyration of a PPO side chain (see FIG. 29). This simplified model assumes that the backbone is a rigid rod, the side chains are Gaussian coils, and that there is no chain overlap. Comparison of the calculated and experimental R_{core} values in Table 11A shows that this model may be reasonable at short core block lengths (N_{c,bb}˜5), but may be an overestimate for longer chains (N_{c,bb}˜40). This behavior resembles that of the Kratky-Porod wormlike chain model, which in turn suggests that, in the micelle core, a sufficiently long bottlebrush PPO can adopt a flexible conformation despite the high grafting density. Finally, for polynorbornene grafted with 1 kDa PPO side chains, a persistence length estimate of ˜10 nm is consistent with a transition from rod-like to coil-like behavior occurring within the interval 5<N_{c,bb}<40. This estimate is higher than the 0.7 nm persistence length of a linear polynorbornene polymer, as expected for a densely grafted bottlebrush.

Accordingly, described herein are synthetic strategies to create bottlebrush poloxamers. The combination of living anionic and ROMP polymerization can afford control over graft length and graft end-group. In some cases, such a combination can allow for quantitative conversion of the macromonomer. A series of hydrophilic bottlebrush poloxamers was synthesized over a range of molecular weights, and the micellization behavior was compared to linear poloxamers. Bottlebrush poloxamers can exhibit a reduced driving force for micellization compared to linear poloxamers, which can be evidenced by a two order of magnitude smaller scaling exponent between Mₙ and CMCₐ.

### Example 12

### Materials

All materials were used as received. The following materials were purchased from Sigma: propylene oxide, (>99.0%), butyl-magnesium chloride, potassium tert-butoxide, 18-crown-6 ether, palladium (10% on activated charcoal), 4-dimethylaminopyridine (>99%), N,N′ diisopropylcarbodiimide (99%), exo-5-norbornene-2-carboxylic acid (97%), methyl ether poly(ethylene glycol) (Mₙ=2000 g/mol), poly(ethylene oxide) (Mₙ=20,000 g/mol), tetrahydrofuran (ACS reagent, 97%, stabilized with 250 ppm BHT), pyridine (>99%), ethyl vinyl ether (stabilized with 0.1% KOH, 99%), benzene (ACS reagent, 99%), sodium chloride (99.5%), potassium chloride (>99%), calcium chloride (anhydrous, 96%), magnesium chloride (97%), HEPES buffer (99.5%), tetrahydrofuran (anhydrous, 99.9%), second generation Grubbs Catalyst® M204, α-cyano-4-hydroxycinnamic acid, and sodium trifluoroacetic acid (98%). The following materials were purchased from Fisher Scientific: methanol (HPLC grade), diatomaceous earth (Celite), dichloromethane (ACS reagent, anhydrous, 99.8%), diethyl ether (anhydrous), and sodium sulfate (anhydrous). SiliaMetS DMT was purchased from Silicycle. Deuterated chloroform (99.8% d) was purchased from Cambridge Isotope Laboratories. Hydrogen gas was purchased from Airgas.

### Example 13

### Anionic Polymerization of Propylene Oxide

Synthesis of tert-butyl-poly(propylene oxide) (t-PPO-OH) was carried out through anionic polymerization. Details of the synthetic procedure can be found, e.g., in Ndoni S et al., Rev. Sci. Instrum. 1995;66(2): 1090-1095; and Hillmyer M A et al., Macromolecules 1996;29(22):6994-7002. Briefly, anionic polymerization was carried out in a water-free and air-free environment under argon. Tetrahydrofuran was dried using an alumina column and used as the solvent. 18-crown-6-ether was used in a 2:1 molar ratio relative to potassium ions to reduce unwanted side reactions and increase conversion of PPO. Propylene oxide was purified over butyl-magnesium chloride and added to the reactor. Potassium t-butoxide was added to initiate the reaction at 25° C., and the reaction was stirred for 72 h. The polymerization was terminated with acidic methanol. The resulting product was then subjected to repeated filtering and rotary evaporation, followed by two hexane/water liquid-liquid extractions in a separatory funnel to remove crown ether and salts from the product. The volume ratio of hexane: water was roughly 1:2; the hexane phase was collected, dried over anhydrous sodium sulfate; and the solvent was removed with a rotary evaporator.

### Example 14

### Hydrogenation of Poly(Propylene Oxide)

An example hydrogenation protocol is as follows. Poly(propylene oxide) (PPO) polymer, faint yellow gel (1.19 g, 1.1 mmol) was put in a round bottom flask with palladium on carbon catalyst (0.27 g) at a 5:1 PPO: Pd/C by mass. The flask was gently rotated to coat the polymer with the catalyst. 10 mL of methanol was added, and the solution was stirred and bubbled with argon for 30 min. Hydrogen gas was collected using a balloon and syringe technique and added to the flask via a needle. A 25 gauge needle was used to displace argon with hydrogen for 1 min. The reaction was stirred at room temperature overnight. The reaction effluent was diluted with methanol and filtered with Celite until the filtrate was clear. Solvent was evaporated using a rotary evaporator to yield a faint yellow gel (72% yield).

### Example 15

### Esterification of PPO-OH

An example esterification protocol is as follows. Hydrogenated PPO (0.83 g, 0.76 mmol) was dried using a vacuum line in a 20 mL scintillation vial. Then, 4-dimethylaminopyridine (DMAP) (0.019 g, 0.16 mmol) and exo-5-norbornene-2-carboxylic acid (0.16 g, 1.15 mmol) were added to the scintillation vial and dissolved in 4.5 mL anhydrous dichloromethane. Meanwhile, N,N′ -diisopropylcarbodiimide (DIC) (0.14 g, 1.14 mmol) was mixed with 1 mL of anhydrous dichloromethane. The DIC solution was then added dropwise to the reaction vial over 3 min while stirring. White precipitate formed within 30 min and the reaction was stirred at room temperature for 7 d. The reaction solution was filtered with a fine filter to remove the precipitate, and the effluent was diluted with hexanes and washed three times with a 0.1 M NaOH solution with addition of a small volume ˜3 mL of saturated NaCl brine. The organic phase was dried over sodium sulfate, concentrated with a rotary evaporator to yield a faint yellow gel with crystals due to excess exo-5-norbornene-2-carboxylic acid. This mixture was then dried in a vacuum oven at 50° C. for 7 d, where the crystals slowly sublimed to leave a yellow gel (90% yield).

### Example 16

### Esterification of PEO-OH

The same reaction protocol as for PPO esterification was performed, except the reaction was run for 48 h. The reaction solution was filtered with a fine filter to remove the precipitate and dried on a rotary evaporator. The product was dissolved in tetrahydrofuran, and the polymer was purified from the excess exo-5-norbornene-2-carboxylic acid by two precipitations in cold diethyl ether to yield a white powder (64% yield).

### Example 17

### Grubbs Catalyst Preparation

Second generation Grubbs catalyst (G2) [(H2IMes)(PCy3)Cl2Ru=CHPh] was purchased from MilliporeSigma and converted to Grubbs third generation catalyst (G3) (see, e.g., Love J A, et al., Angew. Chemie—Int. Ed. 2002;41(21):4035-4037). G2 (1.01 g, 1.2 mmol) was mixed with pyridine (3.0 g, 36 mmol) in a 50 mL round bottom flask and stirred in air for 10 minutes. An immediate color change from dark red to dark green was observed. 20 mL of pentane was layered on top, and the flask was placed in a −20° C. freezer overnight. The product (a green powder) was recovered via vacuum filtration and washed with excess pentane (98% yield).

### Example 18

### Sequential Ring Opening Metathesis Polymerization (ROMP)

All ROMP reactions were conducted in a glovebox with an argon atmosphere ([O_{2]<10} ppm; [H₂O]<10 ppm) with a monomer concentration of approximately 0.05 M in anhydrous dichloromethane and were stirred at room temperature for 10 min. Fresh G3 stock solution (dark green) and monomer stock solutions were prepared with precisely known molar concentrations. To initiate polymerization, a precise volume of G3 stock solution was added to the first monomer solution via a gas tight syringe, such that the molar ratio of monomer to catalyst was equal to the target degree of polymerization. The solution quickly faded from dark green to rust brown upon initiation. The reaction was stirred for 10 min, then a minimal volume of the reaction was taken as an aliquot and quenched with a 1:1 (vol) mixture of ethyl vinyl ether: dichloromethane. The second monomer solution was added to the reaction vessel, again such that the molar ratio of monomer to catalyst was equal to the target degree of polymerization. The reaction was taken out of the glovebox and quenched by stirring with an equal volume of the ethyl vinyl ether: dichloromethane mixture for 10 min. G3 catalyst was removed by diluting the reaction with ˜50 mL dichloromethane and stirring with several scoops of SiliaMetS-DMT (silicycle) for ˜2 h until the supernatant is clear. The silicycle with bound G3 was removed by filtering through a column of Celite. The effluent was dried with a rotary evaporator, dissolved in benzene and freeze dried to recover the polymer as a white powder. Typical yields are ˜80%.

### Example 19

### Polymer Characterization

¹H NMR: All NMR samples were prepared in deuterated chloroform at a sample concentration between 10-20 mg/mL. All spectra were collected using a Bruker Avance III HD-500 MHz spectrometer with a 5 mm Prodigy TCI cryoprobe. Chemical shifts are reported in parts per million and were referenced to the residual chloroform peak at 7.26 ppm. ¹H NMR was used to quantify conversion, assess purity, and confirm polymer composition.

Size Exclusion Chromatography (SEC): All SEC samples were prepared in tetrahydrofuran at a concentration of 5 mg/mL and filtered with a 0.2 um PTFE filter. An instrument equipped with 2 phenogel columns and a Wyatt Dawn Heleos II multi-angle light scattering detector was used to obtain molecular weights and dispersities. The reported refractive index increment (dn/dc) of PEO and PPO in THF are 0.068 mL/g and 0.087 mL/g, respectively. The dn/dc of all diblocks was taken as the weighted average of these components based on the targeted composition.

MALDI-ToF: To prepare MALDI samples, a 30 mg/mL solution of α-cyano-4-hydroxycinnamic acid (CHCA), a 3 mg/mL polymer solution, and a 1 mg/mL sodium trifluoroacetic acid (NaTFA) solution, all in tetrahydrofuran were prepared. The CHCA and polymer solutions were mixed in a volume ratio of 2:1 and a drop of the NaTFA solution was added. An AB SCIEX TOF/TOF 5800 instrument was used in reflector mode. For every MALDI-ToF spectra collected, the laser power of the first shot was deliberately set too low to transfer the polymers into the gas phase. The laser power was then ramped in small steps until a clear spectrum was observed. Thus, all spectra reported were taken near the minimal laser power required, minimizing likelihood of sample degradation.

Micelle solution preparation: Aqueous buffer was prepared according to an established recipe (see, e.g., Kim M et al., Biomacromolecules 2017;18(7):2090-2101). The buffer solution was 140 mM NaCl, 5 mM KCl, 2.5 mM CaCl₂, 2 mM MgCl₂, and 10 mM HEPES, which was prepared in milliQ water (18 Ω); and pH adjusted to 7.2 with NaOH. Micelle stock solutions were prepared via direct dissolution, and a vortexer was used to facilitate dissolution. To ensure complete dissolution, solutions were held at 4° C. for 1 h. Each series of micelle solutions was then prepared via serial dilution and were annealed at 37° C. overnight prior to measurements.

Dynamic Light Scattering (DLS): DLS experiments were performed with a Brookhaven BI-200SM instrument equipped with a 637 nm laser and an adjustable goniometer. All measurements were conducted at 37° C. in a refractive index matching bath. All solutions were filtered with a 0.2 um GHP filter into clean glass test tubes (200 mm×7 mm with 5 mm inner diameter) and sealed with parafilm. All excess scattering intensity measurements were performed at a detector angle of 90°, a laser power of 5 mW, and an aperture setting of 400 nm. Intensity autocorrelation functions, g₂(t) were recorded for a minimum of 5 minutes.

The Siegert relation was applied to convert g₂(t) to the electric field autocorrelation function, g₁(t). g₁(t) was then fit to a second-order cumulant model (Eq. 7.1) and a two-exponential model (Eq. 7.2), and the residuals were compared (FIG. 24A-D, FIG. 25A-D, and FIG. 26D):

$${g}_{1} ( t ) = exp ( - 2 Γ t + {k}_{2} {t}^{2} ) and \qquad ( 7.1 )$$

$${g}_{1} ( t ) = {f}_{1} exp ( - 2 {Γ}_{1} t ) + {f}_{2} exp ( - 2 {Γ}_{2} t ) . \qquad ( 7.2 )$$

The mutual diffusion coefficient (Dₘ) of a species can be obtained by dividing the decay coefficient (I′ in the equations above) by the scattering vector q given by q=(4πn/λ)sin(θ/2), where n is the refractive index of the medium, λ is the wavelength of the laser, and θ is the angle of detection. For dilute solutions, the mutual diffusion coefficient (Dₘ) and the tracer diffusion coefficient (Dₜ) are roughly the same. Dₜ can then be related to the hydrodynamic radius via the Stokes-Einstein equation.

Cryo-TEM: For sample vitrification, 200 mesh lacey carbon copper grids (Ted Pella) were exposed to glow discharge to increase their surface energy. They were then transferred to an environmentally controlled chamber (FEI Vitrobot Mark III), which was held at 25° C. and 100% humidity. 5 μL of a 10 mg/mL polymer solution was then deposited onto the grid and blotted for 7 s. It was then allowed to anneal for 1 s before being plunged into a vat of liquid ethane. The samples were stored in liquid nitrogen prior to imaging. Imaging was performed using an FEI Technai Spirit Bio-Twin TEM at an accelerating voltage of 120 keV. Samples were kept at −176±1° C. during imaging. Images were captured using a magnification of 40000× and an under-focus of approximately −20. Image analysis was performed using ImageJ software, and the built-in enhance local contrast filter was applied to all images. A minimum of 10 objects were measured in each image, and at least two grid locations were included in every analysis.

### Example 20

### In Vitro Characterization of Bottlebrush Polymers Using Muscle Fiber Contractility Testing

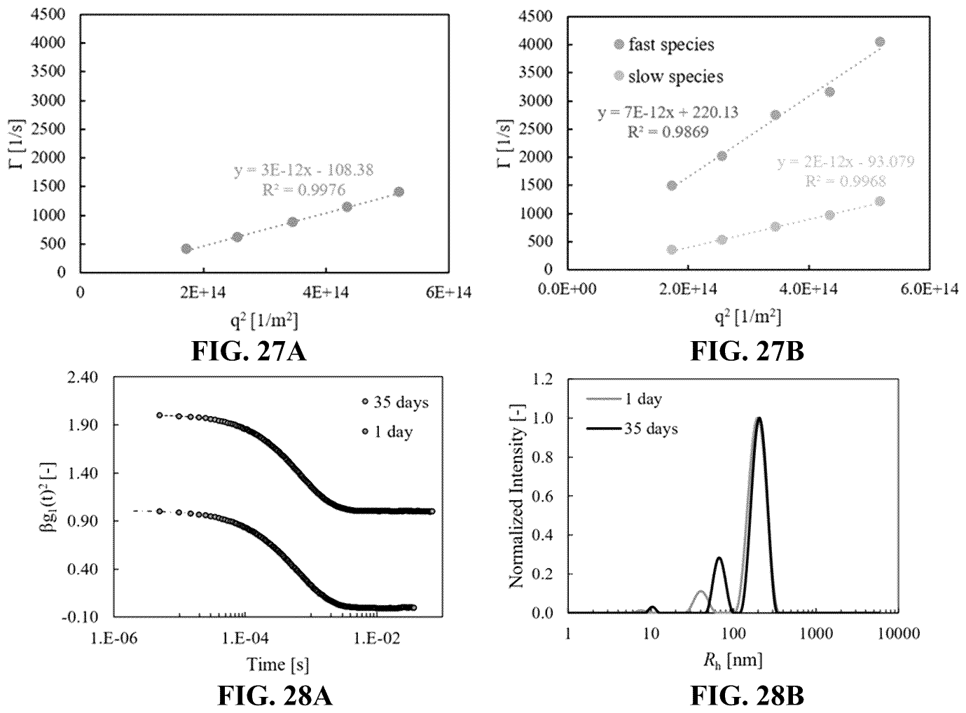

Bottlebrush (BB) polymers having various configurations were obtained (FIG. 30), including the following provided in Table 20A.

TABLE 20A
Summary of materials

| Polymer^{a} | Mₙ [g/mol] | Ð^{b} | wt % PEO |
|---|---|---|---|
| BB-E(45, 10)-b-BB-P(15, 5) | 26200 | 1.07 | 72 |
| BB-E(45, 15) | 30600 | 1.06 | 93 |
| BB-E(8, 80)-b-BB-P(15, 8) | 40900 | 1.08 | 53 |

^{a}BB-E(x, y)-b-BB-P(n, m) nomenclature: the first number is the side chain degree of polymerization, and the second number is the backbone degree of polymerization.
^{b}Ð is the polydispersity of the molecular weight distribution, in which Ð = M_{w}/Mₙ, where M_{w} is the weight averaged molecular weight and Mₙ is the number averaged molecular weight.

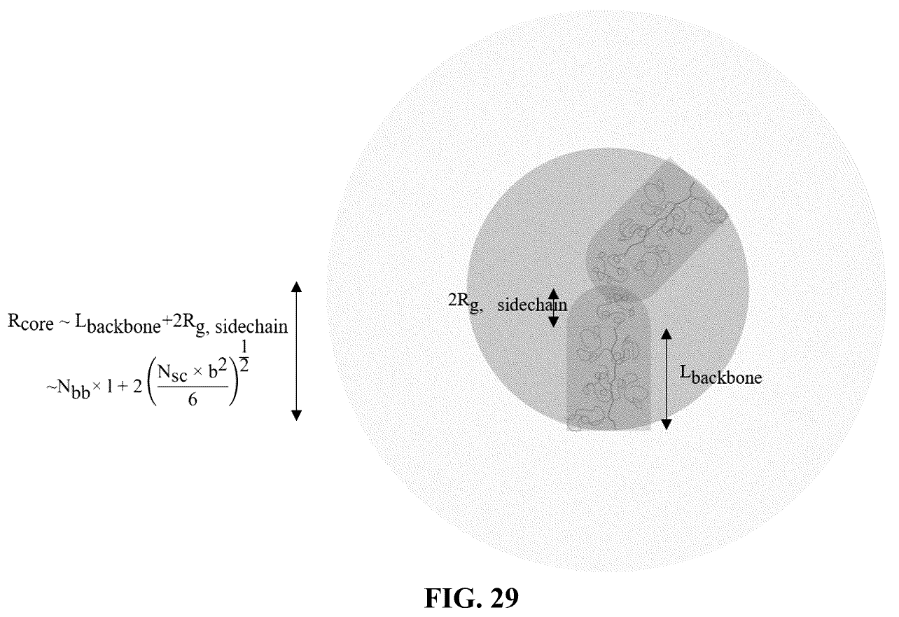

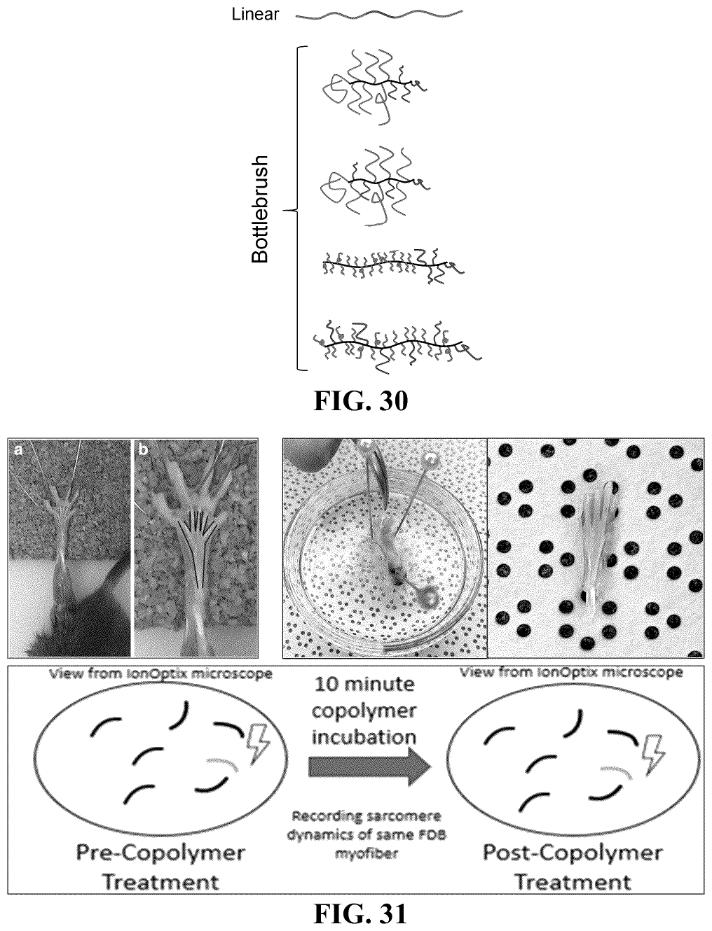

BB polymers were tested by way of in vitro live FDB skeletal muscle fiber contractility testing methods (FIG. 31), in which low dosing of the BB polymer provided enhancement of twitch contractile performance (FIG. 32). The BB polymer was more potent (e.g., at least 5000 times more potent) than P188, a “first-in-class” linear polymer.

### Example 21

### In Vivo Characterization of Bottlebrush Polymers Using Cardiac Stress Test

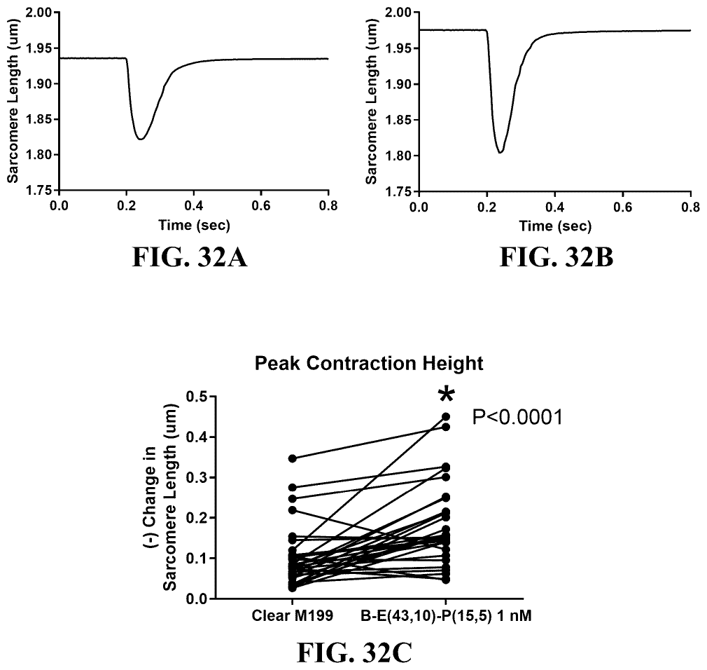

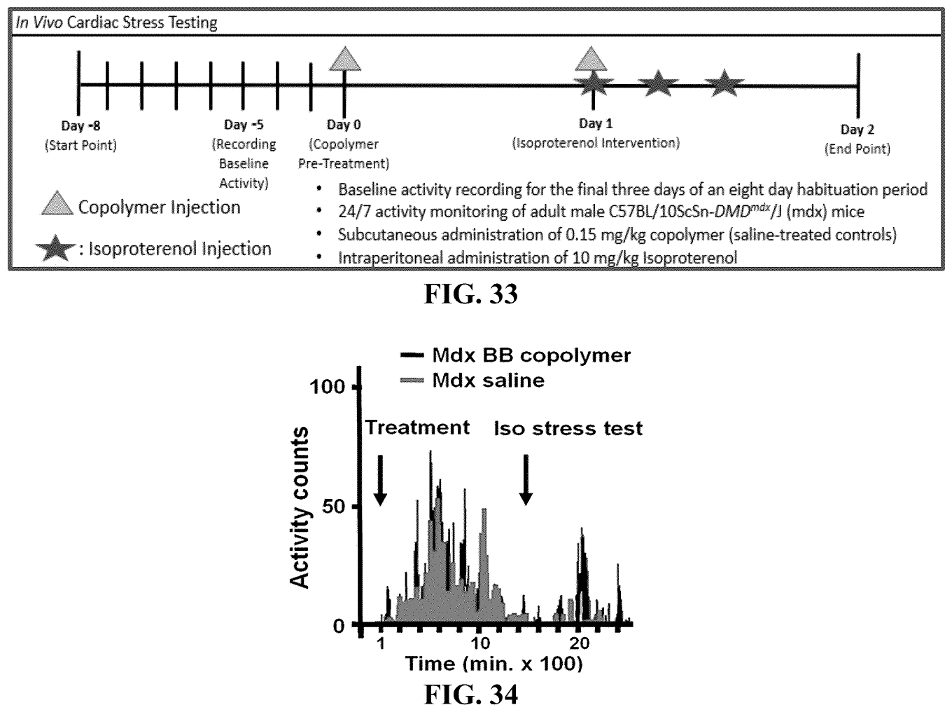

BB polymers were tested by way of in vivo cardiac stress testing methods (FIG. 33), in which pre-treatment with a BB polymer or saline (as control) (at day 0) was then followed by isoproterenol (Iso) intervention (at day 1). In live animal stress testing using dystrophin-deficient (mdx) mice, the BB-treated mice were protected after the Iso stress test, whereas saline-treated mice were not (FIG. 34). In particular, the BB polymer exhibited improved activity (e.g., at more than 3000 times lower dosing) as compared to P188, a “first-in-class” linear polymer.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A polymer comprising a structure of Formula I: 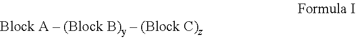
wherein:
each of Block A, Block B, and Block C of the polymer, independently, comprises a backbone polymer comprising one or more repeating units, wherein at least one of the one or more repeating units is covalently linked to a side chain; and
each of y and z is, independently, 0 or 1;
wherein:
a first side chain of Block A is a hydrophilic side chain;
a second side chain of Block B is a hydrophobic side chain; and
a third side chain of Block C is a hydrophilic side chain that is different from the first side chain; and
wherein the polymer is characterized by a molecular weight distribution (Ð) of less than about 1.5, 1.2, 1.15, or 1.1,
wherein Block A comprises poly(ethylene oxide), and
wherein Block B comprises poly(propylene oxide).

2. The polymer of claim 1, wherein the polymer is characterized by one or more of the following:
a number average molecular weight (Mₙ) from about 20 to 400 kDa;
the backbone polymer in the polymer is present in an amount from about 5 to 25 wt. %;
a number of repeating units in the Block A is from about 5 to 200;
a number of repeating units in the Block B is from about 5 to 50;
a number of repeating units in the Block C is from about 5 to 200;
the hydrophilic side chain in the polymer is present in an amount from about 50 to 95 wt. %; and/or
the hydrophobic side chain in the polymer is present in an amount from about 0 to 50 wt. %.

3. The polymer of claim 1, wherein at least one of the one or more repeating units comprises:

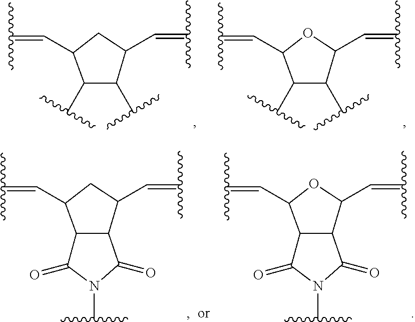

4. The polymer of claim 1, wherein Block A and/or Block B further comprises repeating units that are covalently linked to a polymeric side chain, and optionally wherein the polymeric side chain of Block A comprises a hydrophilic polymeric side chain, and/or optionally wherein the polymeric side chain of Block B comprises a hydrophobic polymeric side chain.

5. The polymer of claim 1, wherein Block A and/or Block C further comprises repeating units that are not covalently linked to a polymeric side chain.

6. The polymer of claim 5, wherein at least one of the repeating units that are not covalently linked to the polymeric side chain comprises: 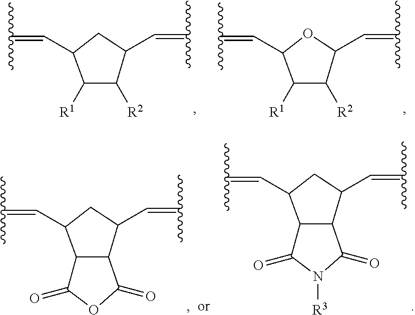
wherein:
each of R¹ and R² is, independently, hydrogen, C₁₋₆ alkyl, or —C(═O)—O—R^{A};
each R^{A} of R¹ and R² is, independently, hydrogen, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl; and
R³ is hydrogen, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl.

7. The polymer of claim 1, wherein at least one of the repeating units that are covalently linked to the side chain comprises: 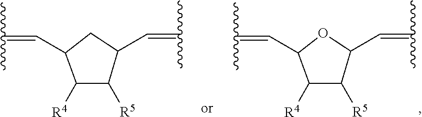
wherein:
each R⁴ is, independently, hydrogen, C₁₋₆ alkyl, the hydrophilic side chain, or the hydrophobic side chain;
each R⁵ is, independently, the hydrophilic side chain or the hydrophobic side chain.

8. The polymer of claim 1, wherein the hydrophilic side chain comprises

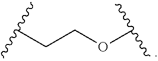 [SMILES: *CCO*]

9. The polymer of claim 1, wherein the hydrophobic side chain comprises 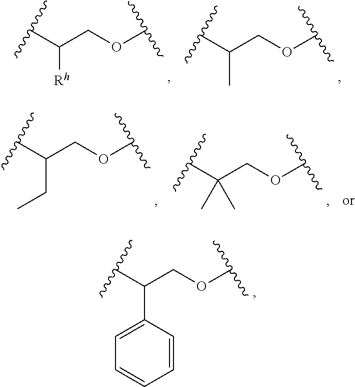
and wherein R^{h} is aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl.

10. The polymer of claim 1, wherein the hydrophilic side chain comprises poly(ethylene oxide) and the hydrophobic side chain comprises poly(propylene oxide).

11. The polymer of claim 1, wherein the hydrophilic side chain and/or the hydrophobic side chain does not include C₂₋₈ alkenyl, and/or wherein at least one of the repeating units are covalently linked to the side chain through a linker, and optionally wherein the linker comprises —C(═O)—O—, —O—C(═O)—, —C(—O)—, or a covalent bond.

12. The polymer of claim 1, wherein Block A comprises more repeating units than a number of repeating units present in Block B.

13. The polymer of claim 1, wherein the polymer comprises a structure of Formula II: 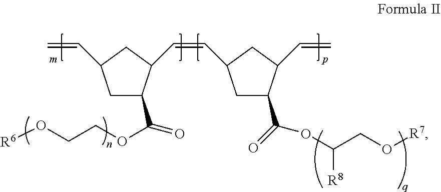
wherein:
each R⁶ is, independently, hydrogen or C₁₋₆ alkyl;
each R⁷ is, independently, hydrogen, C₁₋₁₂ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
each R⁸ is, independently, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
m is 10-200;
n is 5-200;
p is 5-50; and
q is 5-200.

14. The polymer of claim 1, wherein the polymer comprises a structure of Formula IA: 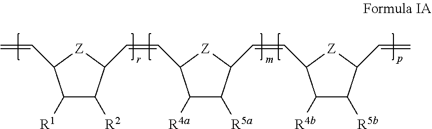
wherein:
Z is oxy (—O—) or C₁₋₆ alkylene;
each of R¹ and R² is, independently, hydrogen, carboxyl, C₁₋₆ alkyl, C₁₋₆ heteroalkyl, C₂₋₁₂ alkoxycarbonyl, or C₅₋₁₈ aryloxycarbonyl, or wherein R¹ and R², when taken together, form an anhydride moiety or a dicarboximide moiety;
each of R⁴a and R⁴b is, independently, hydrogen, C₁₋₆ alkyl, the hydrophilic side chain, or the hydrophobic side chain;
each of R⁵a and R⁵b is, independently, the hydrophilic side chain or the hydrophobic side chain;
m is 10-200;
p is 0-50; and
r is 0-180.

15. A composition comprising a polymer of claim 1 and a pharmaceutically acceptable excipient and optionally further comprising a therapeutic agent.

16. A method of stabilizing an interface, the method comprising delivering a polymer of claim 1 to a solution comprising the interface, optionally wherein the solution further comprises an aqueous solvent, a therapeutic agent, a lipid, or a combination thereof.

17. A method of stabilizing a cell, the method comprising contacting a polymer of claim 1 with a cell or a portion thereof, optionally wherein the cell or the portion thereof comprises a cell membrane or a lipid layer.

18. The method of claim 17, wherein the polymer is provided at a concentration from about 0.001 mg/mL to 30 mg/mL.

19. A method of treating a disease, a disorder, or a condition, the method comprising administering a therapeutically effective amount of a polymer of claim 1 to a subject in need thereof, wherein the disease, the disorder, or the condition is selected from the group consisting of skeletal muscle disorder, sickle cell disease, reperfusion disease or reperfusion injury, ischemia, and cardiovascular disease.

20. A method of preparing a monomer of Formula III: 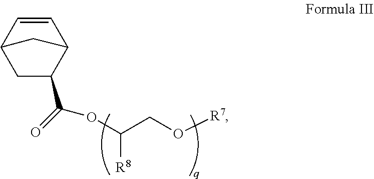
the method comprising:
(i) forming a reaction mixture comprising an alkoxide of R⁷a OH and an epoxide of 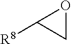
under conditions suitable to prepare a compound of Formula IIIA and a compound of Formula IIIB: 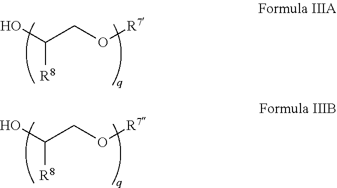
wherein:
each R⁷a is, independently, C₃₋₁₂ alkyl;
each R⁸ is, independently, hydrogen, C₁₋₆ alkyl, aryl, heteroaryl, C₁₋₆ alkyl, C₁₋₆ alkyl-aryl, aryl-C₁₋₆ alkyl, C₁₋₆ alkyl-heteroaryl, or heteroaryl-C₁₋₆ alkyl;
R⁷′ does not include alkenyl;
R⁷″ comprises alkenyl or alkynyl;
each of R⁷′ and R⁷″ is a reacted form of R^{7a}; and
q is 5-200;
(ii) forming a reaction mixture comprising a compound of Formula IIIA, a compound of Formula IB, and a hydrogenation catalyst under conditions suitable to prepare a compound of Formula IA and a compound of Formula IIIC: 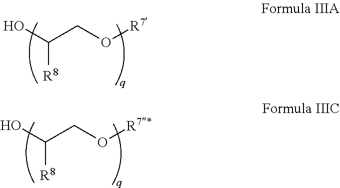
wherein R⁷″* comprises an unsaturated version of R⁷″;
(iii) forming a reaction mixture comprising a compound of Formula IIA, a compound of Formula IIIC, and a compound of 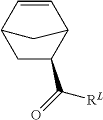
under conditions suitable to prepare a compound of Formula III;
wherein:
each R⁷ is, independently, independently, C₃₋₁₂ alkyl.
